(19) United States
(12) Patent Application Publication
(10) Pub. No.: US 2025/0281569 A1
(43) Pub. Date: Sep. 11, 2025

(54) COMPOSITIONS AND METHODS FOR TREATING NEUROENDOCRINE TUMORS

(71) Applicant: CAMURUS AB, Lund (SE)

(72) Inventor: Fredrik TIBERG, Lund (SE)

(21) Appl. No.: 18/558,623

(22) PCT Filed: Apr. 26, 2022
(86) PCT No.: PCT/SE2022/050404

§ 371 Date: Nov. 2, 2023

(30) Foreign Application Priority Data
May 4, 2021 (SE) 2130118-9

(51) Int. Cl. A61K 38/12; A61K 47/10; A61K 47/14; A61K 47/18; A61K 47/24; A61M 5/20

(52) U.S. Cl. CPC A61K 38/12; A61K 47/10; A61K 47/14; A61K 47/183; A61K 47/24; A61M 5/20

(57) ABSTRACT

This disclosure provides compositions and methods for treating neuroendocrine tumors. A lipid composition comprising 20 mg of octreotide is administered to a patient once every two weeks. The lipid composition provides slow release of octreotide.

### CROSS-REFERENCE TO RELATED APPLICATION

This application claims priority to Swedish Patent Application No. 2130118-9, filed May 4, 2021, which is incorporated herein by reference.

### BACKGROUND

Neuroendocrine tumors (NET) are rare malignant neoplasms that account for about 0.5% of all newly diagnosed malignancies. NET originate from neuroendocrine cells of embryonic origin and can therefore arise in many anatomic sites, such as the gastroenteropancreatic (GEP) tract, the lung, or the central nervous system. Despite certain common morphological and immunohistochemical features, there is significant heterogeneity in the prognosis and treatment strategies according to the primary site, histological differentiation (poorly or well-differentiated), and stage. According to the 2019 version of the World Health Organization classification of tumors, neuroendocrine neoplasms are divided into NET and neuroendocrine carcinomas (NEC) based on their molecular differences. NET are further divided into grades according to their histopathologic characterization and to their proliferation (nuclear antigen Ki-67) index, see table 1:

TABLE 1
Classification and grading criteria for neuroendocrine neoplasms
of the gastrointestinal tract and hepatopancreatobiliary organs

| | | | Mitotic rate | |
|---|---|---|---|---|
| | Differen- | | (mitoses/ | Ki-67 |
| Terminology | tiation | Grade | 2 mm²) | index |
| NET, Grade 1 | Well | Low | <2 | <3% |
| NET, Grade 2 | differentiated | Interme- | 2-20 | 3-20% |
| | | diate | | |
| NET, Grade 3 | | High | >20 | >20% |
| NEC, small-cell | Poorly | High | >20 | >20% |
| type (SCNEC) | differentiated | | | |
| NEC, large-cell | | | >20 | >20% |
| type (LCNEC) | | | | |
| MiNEN | Well or poorly | Variable | Variable | Variable |
| | differentiated | | | |

LCNEC: large-cell neuroendocrine carcinoma;
MiNEN: mixed neuroendocrine-non-neuroendocrine neoplasm;
NEC: neuroendocrine carcinoma;
NET: neuroendocrine tumors;
SCNEC: small-cell neuroendocrine carcinoma

A review of Surveillance, Epidemiology and End Results data showed an incidence of 1.09 cases per 100,000 people per year in 1973 but incidence rate of NET has increased significantly in recent years to approximately 7 cases per 100,000 people per year in the United States (US) in 2012. The majority of patients diagnosed with NET in Western countries are gastroenteropancreatic neuroendocrine tumors (GEP-NET).

GEP-NET may have the following primary tumor locations: stomach, duodenum, jejunum, ileum, pancreas, appendix, cecum, colon, and rectum. GEP-NET have a variable prognosis based on primary site of disease, degree of differentiation and grade, expression of somatostatin receptors, and presence of metastases at diagnosis. Well-differentiated, low-grade GEP-NET have prolonged survival compared to high-grade tumors, and pancreatic NET have more aggressive biology than NET arising in the small intestine.

Surgical resection of the primary and metastatic lesions remains the mainstay of treatment of NET and the only way to obtain a cure. However, resection is often not possible as NET are frequently detected in a more advanced tumor stage when metastases have already developed. In patients with GEP-NET and disease progression, somatostatin analogues (SSAs), mTOR inhibitors, tyrosine kinase inhibitors (sunitinib), alkylating agents, and peptide radionuclide receptor therapy provide treatment options.

Currently, the standard of care for initial treatment of unresectable or metastatic low-grade GEP-NET according to the National Cancer Comprehensive Network is to initiate treatment with SSAs, such as octreotide or lanreotide. Sandostatin® LAR® (octreotide acetate) is marketed as a medicament for symptom control in GEP-NET and lanreotide autogel (ATG) (Somatuline® Depot or Somatuline® Autogel®) is approved in the U.S. for the treatment of patients with unresectable, well- or moderately-differentiated, locally advanced or metastatic gastroenteropancreatic neuroendocrine tumors (GEP-NETs) to improve progression-free survival, and increase time to tumor progression or progression-free survival (PFS) when compared to placebo in treatment-naïve patients with advanced NET. Somatuline is administrated by healthcare providers.

The approved dose of Sandostatin® LAR® for the treatment of advanced midgut NET is 30 mg/month, which is also the maximum approved dose for symptom control. The prescribing information in connection with Sandostatin® LAR® include several warnings relating to increased risk of cardiovascular adverse effects, such as bradycardia and arrhythmia; drug-drug interactions; increased risk of cholelithiasis, hypoglycemia, hyperglycemia, etc. Given that there are only two products available on the market there is still a need for new treatments improving symptom control and/or treatments improving progression-free survival of patients diagnosed with NET, in particular in GEP-NET. Additionally, there is a need for treatments which provide benefits to the patient, and/or caregiver, for example by improving quality of life, ease of administration, e.g., self-administration, etc.

### SUMMARY

This disclosure provides, in part, a lipid composition comprising 20 mg of octreotide or a pharmaceutically acceptable salt thereof for use in treating a gastroenteropancreatic neuroendocrine tumor, and wherein the composition is administered to a patient once every two weeks.

Additionally provided is a pre-filled syringe comprising a lipid composition comprising 20 mg of octreotide or a pharmaceutically acceptable salt thereof for use in treating a gastroenteropancreatic neuroendocrine tumor, and wherein the composition is administered to a patient once every two weeks.

Further provided is an autoinjector comprising a glass compartment containing a lipid composition comprising 20 mg of octreotide or a pharmaceutically acceptable salt thereof for use in treating a gastroenteropancreatic neuroendocrine tumor, and wherein the composition is administered to a patient once every two weeks.

### BRIEF DESCRIPTION OF THE DRAWINGS

FIG. 1 illustrates the clinical trial design

FIG. 2 shows the observed or predicted octreotide plasma concentrations, at steady state, based on predicted pharmacokinetic (PK) parameters and base on a population PK model, and observed PK parameters for Sandostatin LAR (Phase I) to healthy volunteers.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of ordinary skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder, and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The octreotide and its salts (compound) can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of neuroendocrine tumors.

Steady state is the time during which the concentration remains stable or consistent when the drug is given repeatedly. The steady state for the administration of the octreotide compositions disclosed herein is estimated in healthy volunteers to be in the range of 2-3 months, such as about 2 months.

All % are specified by weight herein throughout, unless otherwise indicated. Percent (%) by weight may be abbreviated e.g., as wt. %. Furthermore, the % by weight indicated is the % of the total lipid composition including all the components indicated herein, unless otherwise indicated.

### Methods

The disclosure provides, in part, method of treating neuroendocrine tumors using lipid compositions disclosed herein.

Disclosed herein is a method of treating at least one neuroendocrine tumor, e.g., a gastroenteropancreatic neuroendocrine tumor, comprising, administering to a patient in need thereof a lipid composition comprising 20 mg of octreotide or a pharmaceutically acceptable salt thereof (e.g., chloride salt), wherein the lipid composition is administered to the patient once every two weeks, and wherein octreotide is the sole pharmaceutically active ingredient.

### Compounds

The drug product in the present disclosure is 20 mg/ml octreotide (octreotide chloride). The IUPAC name is (4R,7S,10S,13R,16S,19R)-19-[(2R)-2-amino-3-phenylpropanamido]-10-(4-aminobutyl)-16-benzyl-N-[(2R,3R)-1,3-dihydroxybutan-2-yl]-7-[(1R)-1-hydroxyethyl]-13-[(1H-indol-3-yl)methyl]-6,9,12,15,18-pentaoxo-1,2-dithia-5,8,11,14,17-pentaazacycloicosane-4-carboxamide.

Octreotide has the following chemical structure:

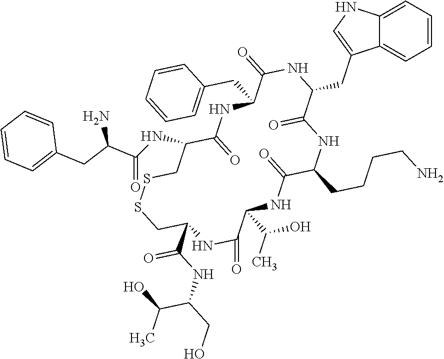 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

and the molecular weight is 1019.3 (free peptide, C₄₉H₆₆N₁₀O₁₀S₂).

The amino acid sequence of octreotide is H2N-D-Phe-Cys-Phe-D-Trp-Lys-Thr-Cys-Thr-ol (Disulfide Bridge Cys2-Cys7).

The lipid composition comprising 20 mg/ml octreotide (concentration based on free peptide), is based on glycerol dioleate and phosphatidylcholine as lipid excipients. Additional excipients in the lipid composition are ethanol (solvent), propylene glycol (co-solvent). The lipid composition optionally includes EDTA and ethanolamine as additional excipients. The water content of the lipid composition at release of the product is not more than (NMT) 1%, determined by USP<921>. The lipid composition comprises octreotide chloride. WO2008/152401 (the full disclosure of which is hereby incorporated herein by reference), see, e.g., examples 4-6 discloses a method for obtaining the octreotide chloride and subsequent formulation as a lipid composition. Further examples are disclosed in WO2012/160213 (the full disclosure of which is hereby incorporated herein by reference), see, e.g., examples 1, 2 and 8. The inactive excipients, i.e., the lipids, glycerol dioleate and phosphatidyl choline, the solvent and co-solvent and the optional excipients EDTA and ethanolamine, and the octreotide chloride adds up to at least 80 wt. %, such as 85 wt. % of the total drug product. The rest are essentially by products, for example, originating from the lipids. Glycerol dioleate (GDO) may also contain monoglycerides (NMT 2%) and triglycerides (NMT 5%). Generally, the GDO used in the lipid composition should contain at least about 93% glycerol dioleate. Phosphatidyl choline may also contain lysophosphatidylcholine (NMT 3%) and triglycerides (NMT 2%). Generally, the phosphatidyl choline used in the lipid composition should contain at least about 94% phosphatidyl choline.

The lipid formulation is provided in a pre-filled syringe, e.g., a glass syringe, equipped with a small needle compared to the comparative product (22 G), and the drug product is not necessary to be reconstituted and considered ready-to-use. Suitable syringes may be those having a volume of about 0.5, about 0.75, about 1.0, about 1.25, and about 1.5 mL. One example is a 1 mL glass syringe, optionally equipped with a small needle compared to the comparative product (22 G).

Comparative drug product used herein are Sandostatin® LAR® (also referred to as octreotide LAR), which contains octreotide as the acetate salt. It is a long-acting octapeptide with pharmacologic properties mimicking those of the natural hormone somatostatin. Sandostatin LAR® Depot (octreotide acetate for injectable suspension) is available in a vial containing the sterile drug product, which when mixed with diluent, becomes a suspension that is given as a monthly intragluteal injection. The excipients are poly(DL-lactide-co-glycolide) and mannitol (E421) in the vial. Carmellose sodium, mannitol (E421), Poloxamer 188 and water for injections as diluent/solvent. The octreotide is uniformly distributed within the microspheres which are made of a biodegradable glucose star polymer, D,L-lactic and glycolic acids copolymer. Sterile mannitol is added to the microspheres to improve suspendability. Sandostatin LAR may also be referred to as octreotide LAR, Oct LAR in the present disclosure. Sandostatin LAR is provided in a vial and in need of at least 30 min reconditioning before added to a syringe for intramuscular injection using a 20 G needle.

SOMATULINE DEPOT (lanreotide) or SOMATULINE Autogel (lanreotide) (ATG) includes lanreotide acetate which is a synthetic cyclical octapeptide analog of the natural hormone, somatostatin. Lanreotide acetate is chemically known as [cyclo S—S]-3-(2-naphthyl)-Dalanyl-L-cysteinyl-L-tyrosyl-D-tryptophyl-L-lysyl-L-valyl-L-cysteinyl-L-threoninamide, acetate salt. Its molecular weight is 1096.34 (base) and its amino acid sequence is:

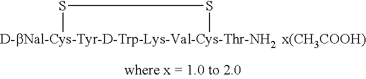

Somatuline DEPOT is a prolonged-release formulation for deep subcutaneous injection. It contains the drug substance lanreotide acetate, water for injection and acetic acid (for pH adjustment). Somatuline ATG also needs to be reconditioned for more than 30 min before subcutaneous injection to a patient via a 18 G/19 G syringe.

### Excipients

The present disclosure also relates to a lipid composition comprising, consisting essentially of, or consisting of 20 mg of octreotide or a pharmaceutically acceptable salt thereof, and glycerol dioleate (GDO), phosphatidyl choline (PC), ethanol, and optionally propylene glycol (PG). The lipid compositions may be substantially non-aqueous.

The lipid compositions may form a depot composition upon contact with an aqueous fluid. The term “depot” relates to the composition which is formed upon exposure of the lipid composition to excess aqueous fluid, e.g., as occurs during numerous parenteral administration routes, such as administration in the subcutaneous tissue of a human patient. The depot typically has a much higher viscosity than the corresponding lipid composition and provides for the gradual release of the octreotide contained within the depot.

### Glycerol Dioleate (GDO)

The lipid composition contains glycerol dioleate (GDO). The GDO may be present in the lipid composition in an amount ranging from 20 to 90 wt. % of the lipid composition (e.g., 30 to 70 wt. %, 33 to 60 wt. %, 43 to 60 wt. %, 38 to 43 wt. %).

Since GDO may be derived from natural sources, there is generally a certain proportion of “contaminant” lipid having other chain lengths, etc. In this context, “pure” GDO is a di-ester of glycerol and two C18:1 fatty acids. Any other diacyl glycerol is considered to be an impurity. In one aspect, GDO, as used herein, indicates any commercial grade of GDO with concomitant impurities (i.e., GDO of commercial purity). These impurities may be separated and removed by purification but, providing the grade is consistent, this is rarely necessary. If necessary, however, “GDO″” may be essentially chemically pure GDO, such as at least 70% pure GDO (e.g., at least 75% pure, at least 80% pure, at least 85% pure, at least 90% pure, at least 93%, at least 95% pure, at least 98% pure, at least 99% pure (area %). In some embodiments, the diglycerides content of the glycerol dioleate is about 92%-100%, such as about 93% to about 99%. The GDO used herein should have an oleic acid content (C18:1) of at least 80% (e.g., at least 85%, at least 90%, at least 95%, at least 98%, at least 99%). In some embodiments, the diglyceride content is about 93% to about 99%, and the oleic acid content is about 88% to about 99% (area %).

Any material used in the lipid composition, including GDO, may potentially include unavoidable trace impurities of metals, optionally including heavy metals. A typical maximum concentration of heavy metals (or elemental impurities) in GDO is 5 ppm.

In alternative embodiments, GDO may be replaced by, or combined with, at least one other lipid, e.g., at least one fatty acid and/or fatty acid ester (lipid). Fatty acids/lipids contain a polar carboxylic acid or ester “head group” with the hydrocarbon chain forming a non-polar “tail” group. Fatty acid esters are esterified fatty acids. Fatty acids or esters used in the lipid composition may be solid or liquid at room temperature and pressure. Examples of non-polar “tail” groups include C6-C32 alkyl and alkenyl groups, which are typically present as long chain carboxylic acids or the esters thereof. These are often described by reference to the number of carbon atoms and the number of unsaturations in the carbon chain. Thus, CX:Z indicates a hydrocarbon chain having X carbon atoms and Z unsaturations. Examples particularly include caproyl (C6:0), capryloyl (C8:0), capryloyl (C10:0), lauroyl (C12:0), myristoyl (C14:0), palmitoyl (C16:0), phytanoyl (C16:0), palmitoleoyl (C16:1), stearoyl (C18:0), oleoyl (C18:1), elaidoyl (C18:1), linoleoyl (C18:2), linolenoyl (C18:3), arachidonoyl (C20:4), behenoyl (C22:0), and lignoceroyl (C24:9) groups. When reference is made herein to the number of carbon atoms in the “chain” or “tail” this number includes the carbon atom of the —C(O)O-moiety, as is conventional in the art.

Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic, or lignoceric acids, or the corresponding alcohols.

The lipid(s) may be saturated or unsaturated. For example, the lipid(s) may comprise at least 1 wt. % unsaturated lipid (based on the total lipid content) (e.g., at least 5 wt. % (5-100%), at least 15 wt. % (15-100%), at least 30 wt. % (30-100%), at least 50 wt. % (50-100%), at least 80 wt. % (80-100%)).

GDO may be replaced or combined with, for example, an edible lipid such as almond oil, avocado oil, butter, canola oil, castor oil, coconut oil, corn oil, cottonseed oil, flaxseed oil, ghee, lard, linseed oil, macadamia oil, margarine, mustard oil, olive oil, palm oil, peanut oil, pumpkin seed oil, rice bran oil, safflower oil, sesame oil, soybean oil, sunflower oil, tea seed oil, vegetable oil, or walnut oil.

One particular example is tocopherol, which may be used to replace GDO.

When GDO is replaced by at least one other lipid, the at least one lipid may be present in the lipid composition in an amount ranging from 20 to 90 wt. % of the lipid composition (e.g., 30 to 70 wt. %, 33 to 60 wt. %, 43 to 60 wt. %, 38 to 43 wt. %). If the lipid composition contains both GDO and at least one additional lipid, the combination may also be present in the lipid composition in an amount ranging from 20 to 90 wt. % of the lipid composition (e.g., 30 to 70 wt. %, 33 to 60 wt. %, 43 to 60 wt. %, 38 to 43 wt. %).

### Phosphatidyl Choline (PC)

The lipid composition also contains phosphatidyl choline (PC). PC is present in an amount ranging from 20 to 80 wt. % of the lipid composition (e.g., 30 to 70 wt. %, 33 to 55 wt. %, 35 to 55 wt. %, 38 to 43 wt. %). Ratios of GDO:PC may be 40:60 to 70:30 (e.g., 45:55 to 55:45, 40:60 to 54:46, 45:55 to 54:46, 47:53 to 53:47). Ratios of around 50:50 (e.g., 49:51 to 51:49) may be highly effective.

In alternative embodiments, PC may be replaced by, or combined with, at least one other phospholipid. The phospholipids may be derived from a natural source. In the case of PC, suitable sources of phospholipids include egg, heart (e.g., bovine), brain, liver (e.g., bovine), and plant sources, including vegetable PC, such as soybean. Such sources may provide one or more constituents of the PC and/or at least one other phospholipid. Any single PC or mixture thereof may be used according to the present disclosure, although egg, heart (e.g., bovine), brain, liver (e.g., bovine), and plant sources, including vegetable PC, such as soybean or any mixture thereof are used in most embodiments disclosed herein.

The PC may be derived from soy. The PC may comprise 18:2 fatty acids as the primary fatty acid component with 16:0 and/or 18:1 as the secondary fatty acid components. These may be present in the PC at a ratio of between 1.5:1 and 6:1. PC having approximately 60-65% 18:2, 10 to 20% 16:0, 5-15% 18:1, with the balance predominantly other 16 carbon and 18 carbon fatty acids may be used, e.g., soy PC. In some embodiments, the PC has a purity of not less than 90%, not less than 92%, not less than 94%, such as about 94%-100% (based on dry weight). The maximum lysophosphatidylcholine content is about 3%, e.g., not more than 3%. The maximum triglycerides content is about 2%, such as not more than 2%. In some embodiments, the PC used in the lipid composition has a purity of about 94%-100% (based on dry weight), and contains not more than 3% lysophosphatidylcholine and optionally not more than 2% triglycerides.

Alternatively, the PC component may comprise synthetic dioleoyl PC (DOPC). The use of DOPC may provide increased stability and so may be used for compositions needing to be stable to long term storage, and/or having a long release period in vivo. Here, the PC component may contain at least 50% synthetic dioleoyl PC (e.g., at least 75% synthetic dioleoyl PC) and even essentially pure synthetic dioleoyl PC. Any remaining PC can be of any sort. DOPC may be more expensive.

Alternative types of phospholipids are synthetic or highly purified PCs, such as dioleoyl phosphatidyl choline (DOPC), may, in alternative embodiments, be used as all or part of the phospholipid component. The synthetic dioleoyl PC may be 1,2-dioleoyl-sn-glycero-3-phosphocholine, and other synthetic PC components include DDPC (1,2-Didecanoyl-sn-glycero-3-phosphocholine); DEPC (1,2-Dierucoyl-sn-glycero-3-phosphocholine); DLOPC (1,2-Dilinoleoyl-sn-glycero-3-phosphocholine); DLPC (1,2-Dilauroyl-sn-glycero-3-phosphocholine); DMPC (1,2-Dimyristoyl-sn-glycero-3-phosphocholine); DOPC (1,2-Dioleoyl-sn-glycero-3-phosphocholine); DPPC (1,2-Dipalmitoyl-sn-glycero-3-phosphocholine); DSPC (1,2-Distearoyl-sn-glycero-3-phosphocholine); MPPC (1-Myristoyl-2-palmitoyl-sn-glycero 3-phosphocholine); MSPC (1-Myristoyl-2-stearoyl-sn-glycero-3-phosphocholine); PMPC (1-Palmitoyl-2-myristoyl-sn-glycero-3-phosphocholine); POPC (1-Palmitoyl-2-oleoyl-sn-glycero-3-phosphocholine); PSPC (1-Palmitoyl-2-stearoyl-sn-glycero-3-phosphocholine); SMPC (1-Stearoyl-2-myristoyl-sn-glycero-3-phosphocholine); SOPC (1-Stearoyl-2-oleoyl-sn-glycero-3-phosphocholine); and SPPC (1-Stearoyl-2-palmitoyl-sn-glycero-3-phosphocholine), or any combination thereof.

Alternative lipid compositions that may be used in the present disclosure are disclosed in WO2016/066655, which is incorporated herein by reference, where lipid slow-release matrices based on triacyl lipids that can form depot compositions on exposure to aqueous fluids without the need for a phospholipid component to be present, though in certain embodiments a phospholipid may also be present.

Alternative embodiments include phosphatidylethanolamine (PE), phosphatidylserine, and phosphatidylinositol, instead of phosphatidyl choline.

Since the lipid compositions are to be administered to a subject, such as a patient, with the inclusion of octreotide, the components should be biocompatible. In this regard, PC (such as soy PC and/or DOPC) and GDO are useful, since they are well tolerated and are broken down in vivo into components that are naturally present in the mammalian body. Appropriate amounts of each component suitable for the combination are those amounts indicated herein for the individual components in any combination. This applies also to any combinations of components indicated herein, where context allows.

When PC is replaced by at least one other phospholipid, the at least one phospholipid may be present in the lipid composition in an amount ranging from 20 to 80 wt. % of the lipid composition (e.g., 30 to 70 wt. %, 33 to 55 wt. %, 35 to 55 wt. %, 38 to 43 wt. %). If the lipid composition contains both PC and at least one additional phospholipid, the combination may also be present in the lipid composition in an amount ranging from 20 to 80 wt. % of the lipid composition (e.g., 30 to 70 wt. %, 33 to 55 wt. %, 35 to 55 wt. %, 38 to 43 wt. %). Ratios of GDO and/or the at least one lipid other than GDO:PC and/or the at least one phospholipid other than PC may be 40:60 to 70:30 (e.g., 45:55 to 55:45, 40:60 to 54:46, 45:55 to 54:46, 47:53 to 53:47). Ratios of around 50:50 (e.g., 49:51 to 51:49) may be highly effective.

### Solvent

The lipid composition also contains the solvent ethanol. In alternative embodiments, the ethanol may be replaced by, or combined with, at least one other biocompatible organic solvent. Since the lipid composition may generate a depot composition following administration (e.g., in vivo), typically upon contact with excess aqueous fluid, it is desirable that this solvent be tolerable to the subject and be capable of mixing with the aqueous fluid, e.g., body fluids, and/or diffusing or dissolving out of the lipid composition into the aqueous fluid. Therefore, solvents having at least moderate water solubility may be used. In some embodiments the lipid compositions include a polar co-solvent.

In alternative embodiments, instead (or in addition to) ethanol, the solvent in the lipid composition comprises, consists essentially of, or consists of at least one solvent selected from the group consisting of: alcohols, amines, amides, and esters. Therefore, the solvent may comprise at least a mono-alcoholic solvent, such as ethanol, propanol, iso-propanol, or mixtures thereof. In one embodiment, the solvent is ethanol. The lipid composition may also comprise, consist essentially of, or consist of a mono-alcoholic solvent (e.g., ethanol) and a polar co-solvent (e.g., propylene glycol). The amount of solvent in the lipid composition may affect several features, including, for example, the viscosity and the rate (and duration) of release. Therefore, the amount of solvent may be at least sufficient to provide a low viscosity mixture but may additionally be determined so as to provide the desired release rate. Typically, a level of 1 to 30% (e.g., 2 to 20%, 2 to 18%, 2 to 16%, 2 to 15%) solvent will provide suitable release and viscosity properties. In some embodiments, the solvent (e.g., EtOH) is at a level of 3-10%, and a co-solvent (e.g., PG) is at a level of 3-10%.

As indicated above, the amount of solvent in the lipid compositions may be at least sufficient to provide a low viscosity mixture (e.g., a molecular solution) of the components of the lipid composition and can be determined for any particular combination of components by standard methods.

The solvent may be a single solvent (e.g., ethanol) or a mixture of suitable solvents (e.g., ethanol and PG) but will generally be of low viscosity. In some embodiments the solvent is a mixture of ethanol and propylene glycol, and no other solvent. The viscosity of the “low viscosity” solvent (single solvent or mixture) may be no more than 18 mPas at 20° C. (e.g., no more than 15 mPas at 20° C., no more than 10 mPas at 20° C., no more than 7 mPas at 20° C.).

WO2012/160213 describes the addition of a polar solvent in addition to a mono-alcoholic solvent results in numerous advantages including reduced viscosity and reduced active agent burst profile. In addition to the aspects described previously for the solvent component, In some embodiments, the solvent comprises a mono-alcoholic solvent (e.g., ethanol) and a polar co-solvent. The term “polar co-solvent” as used herein defines a solvent having a dielectric constant (diel) of at least 28 at 25° C. (e.g., at least 30 at 25° C.) but is not water or any aqueous fluid. Examples include propylene glycol (diel −32), and N-methyl-2-pyrrolidone (NMP, diel −32). The levels of solvent recited herein may apply equally to mixtures of mono-alcoholic solvent and a polar co-solvent unless context permits otherwise.

In another embodiment, the solvent comprises, consists essentially of, or consists of a mixture of a mono-alcoholic solvent and a polar co-solvent. The polar co-solvent may be a di-alcoholic C3-C6 organic solvent, i.e., a C3-C6 organic solvent comprising two hydroxy groups. The di-alcoholic solvent may be propylene glycol. When present, a polar co-solvent may be included at a level of 2 to 12 wt. % of the lipid composition (e.g., 3 to 10 wt. %, 4 to 9 wt. %). This level is counted as part of the ranges recited above for the solvent. In one embodiment, the solvent comprises, consists essentially of, or consists of a mixture of ethanol and propylene glycol (PG), where PG is considered a co-solvent.

Where both an organic mono-alcoholic solvent and a polar co-solvent are present, e.g., ethanol and PG, the ratio of mono-alcoholic solvent to polar co-solvent solvent may be in the range 20:80 to 70:30 (w/w) (e.g., 30:70 to 70:30 (w/w), 40:60 to 60:40 (w/w)). The amounts and ratio of ethanol and PG may have an effect on properties such as release of an active agent, viscosity of the lipid composition, etc., features which are all important characteristics of suitable lipid compositions for subcutaneous injection of an active agent to a patient in need thereof.

In one embodiment, the solvent is present at a level of 1 to 30 wt. % (e.g., 5 to 15 wt. %, 8 to 18 wt. %, 8 to 18 wt. %) and comprises, consists essentially of, or consists of a mixture of ethanol and PG, wherein the ratio of ethanol to PG (w/w) is in the range of 30:70 to 70:30 (w/w) (e.g., 40:60 to 60:40 (w/w)). Additional examples are about 4-10 wt. % ethanol and about 4-10 wt. % PG, e.g., about 5-8 wt. % of each.

Even where a polar co-solvent is present in the lipid composition, the total water level may remain at release of the lipid composition for sale at the levels describes herein (e.g., 1.0 wt. % or less, 0.1 to 1.0 wt. %).

The term “release” in this context means released for administration to a patient, i.e., a pharmaceutical product approved by a regulatory agency, e.g., the FDA. The term release specification means the tests and limits against which raw material, intermediate, and final product are measured prior to use and/or release.

### Optional Component—EDTA

The lipid composition may also optionally contain EDTA (“ethylenediamine tetraacetic acid” or “edetic acid”). As used herein, the term “EDTA” may represent ethylenediammetetraacetic acid as such. Alternatively, EDTA as indicated herein may include ethylenediammetetraacetic acid itself, EDTA analogues, and alkylammonium EDTA salts. “EDTA” herein thus includes “EDTA, analogues thereof, and alkylammonium EDTA salts,” whenever context allows. “EDTA” does not include the disodium salt of EDTA.

Examples of EDTA analogues include:
Iminodiacetic acid (IDA)—(NH(CH₂CO₂H)₂;
Nitrilotriacetic acid (NTA)—N(CH₂CO₂H)₃;
Pentetic acid*—N(CH₂CO₂H)₂CH₂CH₂N(CH₂CO₂H)CH₂CH₂N(CH₂CO₂H)₂;
Egtazic acid—N(CH₂CO₂H)₂CH₂CH₂OCH₂CH₂OCH₂CH₂N(CH₂CO₂H)₂;
NOTA—[N(CH₂CO₂H)CH₂CH₂]3; and
DOTA—[N(CH₂CO₂H)CH₂CH₂]4.
* Also known as “DTPA”

EDTA analogues and alkylammonium salts thereof are further disclosed and described in WO 2018/060212, which is hereby incorporated herein by reference.

The alkylammonium salt(s) of EDTA is provided by contacting the EDTA or analogue thereof with a suitable alkylamine, examples are:
Ethanolamine “ETA” (NH₂(CH₂CH₂OH));
Diethanolamine “DiETA” (NH(CH₂CH₂OH)₂);
meglumine (NH(CH₃)CH₂(CHOH)₄CH₂OH));
tris-hydroxymethylamine “TRIS” (N(CH₂OH)₃);
ethylenediamine (NH₂CH₂CH₂NH₂); or
serinol (NH₂CH(CH₂OH)₂).

The mass of the alkylammonium cation of Formula (I) may be below 500 amu (e.g., below 350, below 250 amu). Salts of EDTA containing the ethanolammonium ion (HOCH₂CH₂NH₃) may be used. The EDTA salt may be a salt of EDTA with ethanolamine (ETA) (e.g., EDTA with ETA only).

The lipid composition may also contain EDTA salts comprising an anion of EDTA and at least one alkylammonium cation of the suitable alkylamine as previously described.

The provision of EDTA and/or a structural analogue thereof and a suitable alkylamine, e.g., ethylenediamine tetraacetic acid and ethanolamine, or ethylenediamine tetraacetic acid and diethanolamine, is believed to enable the solubilization of EDTA or a structural analogue thereof by forming an alkylammonium salt of EDTA or structural analogues thereof. Contrary to the inorganic sodium salt of EDTA, i.e., EDTA disodium salt, the provision of the alkylamine and the EDTA or structural analogues thereof enables substantial amounts of EDTA in the solution of lipid components. Further details are available in WO 2018/060212.

### Optional Excipients

Besides the octreotide or pharmaceutically acceptable salt thereof, the lipid composition may contain additional excipient(s).

The octreotide dissolved in the lipid composition, may gain stability (both storage and in vivo stability) by certain stabilizing additives. Such additives include, but are not limited to, sugars (e.g., sucrose, trehalose, lactose, etc.), polymers (e.g., polyols such as carboxy methyl cellulose), amino acids (such as methionine, glutamate, lysine, etc.), lipid-soluble acid components such as HCl, anionic lipids, and/or surface active agents (such as dioleoyl phosphatidyl glycerol (DOPG), palmitoyloleoyl phosphatidylglycerol (POPG), and oleic acid (OA)). Although all of the above are possible ways to alter the properties of lipid compositions disclosed herein the EDTA is generally deemed as sufficient to achieve the necessary stability of the lipid composition.

Single-dose formats, such as pre-filled syringes comprising lipid compositions disclosed herein, must remain stable and potent in storage prior to use but are disposable after the single use. The substantially non-aqueous lipid compositions have enhanced storage stability at elevated temperatures, such as at 25° C. or even 40° C. This offers advantages in terms of ease of transportation and storage (no need for refrigeration). A single dose format of the lipid composition may have stability such that after storage for 2 months at 25° C. (with air in head space), the assayed octreotide concentration is at least 95% that of the initial assayed octreotide concentration, and after 3 months, the assayed octreotide concentration is at least 90% that of the initial assayed octreotide concentration, the lipid composition disclosed herein generally has an acceptable storage stability of twelve months or longer.

A single dose format of the lipid composition may have a stability such that after storage for 2 months at 40° C. (with air in head space), the assayed octreotide concentration is at least 85% that of the initial assayed octreotide concentration, and after 3 months, the assayed octreotide concentration is at least 80% that of the initial assayed octreotide concentration.

Therefore, the lipid compositions can optionally contain an antimicrobial or microbial-static agent, which includes bacteriostatic agents and preservative. Such agents include benzalkonium chloride, m-cresol, benzyl alcohol, or other phenolic preservatives. Typical concentrations as known in the art can be used. In most aspects and embodiments, there are no antimicrobial or microbial-static agents added.

These additional components, if present, may be present in an amount of 0 to 5 wt. % (e.g., 0.01 to 5 wt. %), such as no more than 2% by weight, or no more than 1% by weight.

In some embodiments the lipid composition comprises no additional excipient, only EDTA and an alkylamine.

### Water Content

It is difficult to eliminate all traces of water (especially from the raw materials). Even if essentially water-free formulations could be achieved, lipid compositions described in this disclosure will typically be stored in ready-to-use form, e.g., in syringes and possibly under refrigerated conditions. Syringes are often not completely air-tight meaning that the level of water in the lipid composition may increase to an appreciable level over time, e.g., over months, even if the initial level of water is insignificant.

The initial absolute level of water in the lipid composition may be between 0 to 1.0 wt. % (e.g., less than 1.0 wt. %, less than 0.8 wt. %, less than 0.5 wt. %). For example, the level of water may be in the range of 0.1 to 0.9 wt. %, such as 0.2 to 0.8 wt. %. These levels refer to the absolute level of water and not added levels of water. Any unavoidable trace of water present within the components of the lipid composition is included in this stated level of water. After 3 months of storage, the absolute water level may be no more than 1.5 wt. %. Absolute levels of water can be measured by methods well known in the art, such as Karl Fischer titration. For example, the water content may be measured according to the procedure in United States Pharmacopoeia (USP 40—NF 35, USP <921> Water determination, Method 1a).

The lipid composition has a water content of between 0 to 1.0 wt. % (e.g., less than 1.0 wt. %, less than 0.8 wt. %, less than 0.5 wt. %) when the lipid composition product is released for sale (e.g., released for administration to a patient).

### Embodiments

The present disclosure relates to a method of treating at least one neuroendocrine tumor (NET) comprising, consisting essentially of, or consisting of administering to a patient in need thereof a lipid composition comprising 20 mg of octreotide or a pharmaceutically acceptable salt thereof, wherein the lipid composition is administered to the patient once every two weeks.

The lipid composition, which is described herein, may be administered to the patient once every two weeks as a single unit-dose, or in multi-dose formats.

The at least one neuroendocrine tumor may be a gastroenteropancreatic neuroendocrine tumor (GEP-NET).

The lipid composition may be administered to the patient by subcutaneous injection. The lipid composition may be administered to the patient by a syringe, a pre-filled syringe, an autoinjector, or a pen injector.

The lipid composition may be administered to the patient not more than once every two weeks (or not more than once every week), and in a volume of about 1 mL.

The lipid formulation may be provided in a pre-filled syringe, e.g., a 1 mL glass syringe, equipped with a small needle compared to the comparative product (22 G), and the drug product is not necessary to be reconstituted and considered ready-to-use.

The bioavailability of octreotide or a pharmaceutically acceptable salt thereof has been shown to be higher for a lipid composition of the present disclosure administered subcutaneously than for Sandostatin® LAR® which is administered as an intramuscular injection. Completed Phase I clinical trials showed that octreotide release from a lipid composition of the present disclosure had a rapid onset of action, with an octreotide Cₘₐₓ observed within approximately 4 to 24 hours after dosing. See Tiberg et al., British J. Clin. Pharmacol. 80(3):460-472 (2015). Thereafter, the plasma concentration slowly declined over time with therapeutic drug levels maintained for approximately 4 weeks, resulting in observable suppression of insulin-like growth factor-1 over 4 weeks after dosing. Dose-proportional PK was observed in the dose range of 10 to 30 mg of the lipid composition. The bioavailability of octreotide was approximately 5 times higher for a lipid composition of the present disclosure than for Sandostatin® LAR®.

The Phase 2 trial showed that switching from Sandostatin® LAR® (10 mg, 20 mg, or 30 mg) to a lipid composition comprising octreotide administered subcutaneously (20 mg once monthly or 10 mg every 2 weeks) was associated with maintenance or decrease in insulin-like growth factor-1 levels (as compared to pre-switch values) and in maintenance of growth hormone levels in patients with acromegaly, and maintenance or improvement of symptom control, as measured by flushing episodes and bowel movements, in patients with NET. See Pavel et al., Cancer Chemotherapy and Pharmacology (2019) 83:375-385. The PK data from the trial confirmed the results from the trials in healthy volunteers and showed a higher exposure of octreotide after treatment with a lipid composition of the present disclosure than after treatment with Sandostatin® LAR®.

Safety, including local injection-site tolerability, was investigated in all trials. The adverse event (AE) profile seen with a lipid composition of the present disclosure was consistent with what has been recorded for Sandostatin® LAR® and octreotide immediate release (IR), with transient and mild to moderate GI events being the most frequently reported AEs.

For each administration of the lipid composition of the present disclosure, the patient may have a maximum blood plasma concentration (Cₘₐₓ) of octreotide (at steady state) of less than 45 ng/ml (e.g., less than 40 ng/ml, less than 35 ng/ml, less than 30 ng/ml), such as 3-45 ng/ml, such as 4-40 ng/ml, such as 5-35 ng/ml. The Cₘₐₓ of octreotide at steady state is determined after 2 months of treatment. The concentration of octreotide may be determined using ultra high-performance liquid chromatography-tandem mass spectrometry, as described in Karnes et al., Journal of Chromatography B, 879 (2011) 2081-2088. Service labs, such as PPD laboratories (https://www.ppd.com/our-solutions/ppd-laboratories/bioanalytical-lab/), may be used.

The patient may also have an average blood plasma concentration (C_{AV}) of octreotide (at steady state) of 3-24 ng/ml (e.g., 4-20 ng/ml, 5-15 ng/ml, 5-10 ng/ml), for each administration of the lipid composition.

The patient may also have a plasma concentration AUC of octreotide (at steady state) of 1300-6700 ng*h/ml (e.g., 1700-5000 ng*h/ml, 1700-3400 ng*h/ml), such as 1550-3500 ng*h/ml, such as 1600-3450 ng*h/ml, such as 1600-3400 ng*h/ml, for each administration of the lipid composition.

Administration of the lipid composition disclosed herein provides a drug depot comprising octreotide in the patient, wherein the depot may provide a release of octreotide to the patient over about two weeks and wherein the patient may have blood plasma levels of octreotide of at least about 3 ng/ml (e.g., at least about 4 ng/ml, at least about 5 ng/ml, at least about 6 ng/ml), for each administration of the lipid composition. The depot may be in the subcutaneous tissue of the patient.

The lipid composition of the present disclosure may provide a progression-free survival (PFS) of at least 16 months (e.g., at least 17 months, at least 18 months, at least 19 months, at least 20 months, at least 21 months, at least 22 months, at least 23 months). PFS is defined as the time from the date of the first administration to the patient of the lipid composition of the present disclosure to the date of the first disease progression, determined according to Response Evaluation Criteria in Solid Tumors version 1.1 (RECIST 1.1), or death to any cause, which occurs first.

The lipid composition of the present disclosure may provide an improved progression-free survival (PFS) compared to Sandostatin® LAR® (30 mg administered intramuscularly every 4 weeks). The present disclosure may also provide an improved or about the same PFS as Somatuline® Depot (Somatuline® Autogel) (120 mg administered subcutaneously every 4 weeks), again PFS is evaluated by Response Evaluation Criteria in Solid Tumors version 1.1 (RECIST 1.1). For example, embodiments of the present disclosure may provide an improved PFS compared to Sandostatin® LAR® (30 mg administered intramuscularly every 4 weeks) and/or an increased or about the same PFS as Somatuline® Depot (Somatuline® Autogel) (120 mg administered subcutaneously every 4 weeks) in patients with, for example, unresectable/metastatic and well-differentiated gastroenteropancreatic neuroendocrine tumors (GEP-NET).

The lipid composition administered once every second week and comprising 20 mg octreotide may provide an improved progression-free survival (PFS) compared to placebo or Sandostatin® LAR® (30 mg administered intramuscularly every 4 weeks), and/or increased or about the same progression-free survival as Somatuline® Depot (Somatuline® Autogel) (120 mg administered subcutaneously every 4 weeks), wherein the progression-free survival is determined radiologically according to Response Evaluation Criteria in Solid Tumors version 1.1 (RECIST 1.1).

The lipid composition administered once every second week and comprising 20 mg octreotide may provide an improved overall response rate (ORR) compared to Sandostatin® LAR® (30 mg administered intramuscularly every 4 weeks) and/or Somatuline® Depot (Somatuline® Autogel) (120 mg administered subcutaneously every 4 weeks) as determined according to Response Evaluation Criteria in Solid Tumors version 1.1 (RECIST 1.1). The overall response rate may be selected from the group consisting of complete response and/or partial response.

The lipid composition of the present disclosure may provide an improved disease control rate (DCR) compared to Sandostatin® LAR® (30 mg administered intramuscularly every 4 weeks) and/or Somatuline® Depot (Somatuline® Autogel) (120 mg administered subcutaneously every 4 weeks) as determined according to Response Evaluation Criteria in Solid Tumors version 1.1 (RECIST 1.1).

The lipid composition of the present disclosure may provide an improved time to tumor response rate compared to Sandostatin® LAR® (30 mg administered intramuscularly every 4 weeks) and/or Somatuline® Depot (Somatuline® Autogel) (120 mg administered subcutaneously every 4 weeks) as determined according to Response Evaluation Criteria in Solid Tumors version 1.1 (RECIST 1.1).

The lipid composition of the present disclosure may provide an improved overall survival rate compared to Sandostatin® LAR® (30 mg administered intramuscularly every 4 weeks) and/or Somatuline® Depot (Somatuline® Autogel) (120 mg administered subcutaneously every 4 weeks) as determined by overall survival rates (i.e., the time from the date of the first administration to the date of death of the patient).

The lipid composition may be administered to the patient once every two weeks until the patient experiences progressive disease (PD), determined radiologically according to Response Evaluation Criteria in Solid Tumors version 1.1 (RECIST 1.1), whereupon the lipid composition may then be administered to the patient once every week. It has been reported that increasing the dose intensity or the dose frequency of somatostatin analogues, such as octreotide, is well tolerated overall, suggesting that shortening the dose interval from once every two weeks to once every week of the lipid composition of the present disclosure is not likely to constitute an increased risk to patients.

The patient may have a maximum blood plasma concentration (Cₘₐₓ) of octreotide (at steady state) of less than 45 ng/ml (e.g., less than 40 ng/ml, less than 35 ng/ml, less than 30 ng/ml), such as 3-45 ng/ml, such as 4-40 ng/ml, such as 5-35 ng/ml, for each once every administration.

The patient may have an average blood plasma concentration (C_{AV}) of octreotide (at steady state) of 6-40 ng/ml (e.g., 7-30 ng/ml, 7-25 ng/ml), for each once every week administration.

The patient may also have an average blood plasma concentration (C_{AV}) of octreotide (at steady state) of 4-20 ng/ml for each once every week administration.

The patient may have a plasma concentration AUC of octreotide (at steady state) of 1000-6700 ng*h/ml (e.g., 1000-5900 ng*h/ml, 1200-5000 ng*h/ml, 1200-4200 ng*h/ml), such as 1550-3500 ng*h/ml, such as 1600-3450 ng*h/ml, such as 1600-3400 ng*h/ml, for each once every week administration.

Administration of the lipid composition may provide a drug depot comprising octreotide in the patient, wherein the depot may provide a release of octreotide to the patient over about 1 week and wherein the patient may have blood plasma levels of octreotide of at least about 5 ng/ml (e.g., at least about 7 ng/ml, at least about 8 ng/ml, at least about 10 ng/ml), for each once every week administration.

Compared to Sandostatin® LAR®, which is administered as an intramuscular (IM) injection and which needs to be reconstituted before injection, the lipid composition of the present disclosure may be provided in a pre-filled syringe with no need for reconstitution. The lipid composition of the present disclosure also offers the option of self- or partner-administration and may be easier to handle and to administer, thereby potentially improving patient convenience and care.

The method of administering the lipid composition of the present disclosure as a small-volume subcutaneous injection in, for example, a pre-filled syringe (i.e., eliminating the need for reconstitution) with, for example, a thin needle may provide a ready-to-use, long-acting octreotide formulation, with a higher octreotide bioavailability than Sandostatin® LAR®, and may lead to improved quality of patient care and treatment convenience.

The present disclosure may not be appropriate for patients with long QT syndrome, a family history of idiopathic sudden death or congenital long QT syndrome, or any of the following:

Risk factors for Torsades de Pointes, including uncorrected hypokalemia or hypomagnesemia, history of cardiac failure, or history of clinically significant/symptomatic bradycardia; Treatment with concomitant medication(s) with a “known risk of Torsades de Pointes” per www.qtdrugs.org that cannot be discontinued or replaced with safe alternative medication at least 7 days or 5 half-lives (whichever is longer) before the first administration of the lipid composition of the present disclosure; or a baseline QTc interval corrected by Fridericia's formula (QTcF) of >450 msec for male patients and >470 msec for female patients before the first administration of the lipid composition of the present disclosure.

Upon administration of the lipid composition of the present disclosure to a patient in need thereof, the dosing of the lipid composition may not need to be interrupted for male patients having an average QTcF of 450 msec to 480 msec and for female patients having an average QTcF of 470 msec to 480 msec.

Upon administration of the lipid composition of the present disclosure to a patient in need thereof, for male and female patients having an average QTcF of 481 msec to 500 msec, the dosing of the lipid composition may need to be interrupted/paused/delayed until the average QTcF of the patient falls below 481 msec, whereupon the dosing of the lipid composition may resume.

### EXEMPLARY EMBODIMENTS

In some embodiments, disclosed herein is a method of treating at least one neuroendocrine tumor comprising, consisting essentially of, or consisting of, administering to a patient in need thereof a lipid composition comprising 20 mg of octreotide or a pharmaceutically acceptable salt thereof, herein the lipid composition is administered to the patient once every two weeks.

In some embodiments, the lipid composition is administered as a unit dose.

In some embodiments, the at least one neuroendocrine tumor is a gastroenteropancreatic neuroendocrine tumor.

In some embodiments, the octreotide or pharmaceutically acceptable salt thereof in the lipid composition is octreotide chloride.

In some embodiments, the octreotide or pharmaceutically acceptable salt thereof in the lipid composition is octreotide chloride, and the octreotide chloride is the only active ingredient in the lipid composition.

In some embodiments, the lipid composition comprises, consists essentially of, or consists of the octreotide or pharmaceutically acceptable salt thereof (e.g., octreotide chloride), glycerol dioleate, phosphatidylcholine, and ethanol.

In some embodiments, the lipid composition comprises, consists essentially of, or consists of the octreotide or pharmaceutically acceptable salt thereof (e.g., octreotide chloride), glycerol dioleate, phosphatidylcholine, ethanol, and propylene glycol.

In some embodiments, the lipid composition comprises, consists essentially of, or consists of the octreotide or pharmaceutically acceptable salt thereof (e.g., octreotide chloride), glycerol dioleate, phosphatidylcholine, ethanol, and EDTA.

In some embodiments, the lipid composition comprises, consists essentially of, or consists of the octreotide or pharmaceutically acceptable salt thereof (e.g., octreotide chloride), glycerol dioleate, phosphatidylcholine, ethanol, propylene glycol, and EDTA.

In some embodiments, the lipid composition comprises, consists essentially of, or consists of the octreotide or pharmaceutically acceptable salt thereof (e.g., octreotide chloride), glycerol dioleate, phosphatidylcholine, ethanol, propylene glycol, EDTA, ethanolamine and/or diethanolamine.

In some embodiments, at least 80 wt. % of the lipid composition (e.g., at least 85 wt. %, at least 90 wt. %, at least 95 wt. %) may comprise the octreotide or pharmaceutically acceptable salt thereof (and any other optional active ingredients) and lipid(s), phospholipid(s), solvents, and, if present, EDTA (allowing for any impurity inherent in these components). For example, at least 85 wt. % of the lipid composition (e.g., 86 wt. %, 87 wt. %, 88 wt. %, 89 wt. %) may comprise the octreotide (e.g., octreotide chloride), at least one lipid (e.g., glycerol dioleate), at least one phospholipid (e.g., phosphatidylcholine), at least one biocompatible organic solvent (e.g., ethanol and propylene glycol), and EDTA. These lipid compositions may have an initial (at release of the drug product) water of content of less than 1.0 wt. % (e.g., less than 0.9 wt. %, less than 0.8 wt. %, less than 0.7 wt. %, less than 0.6 wt. %, less than 0.5 wt. %), and/or a water content of less than 1.0 wt. % (e.g., less than 0.9 wt. %, less than 0.8 wt. %, less than 0.7 wt. %, less than 0.6 wt. %, less than 0.5 wt. %) at the release of the lipid composition product for sale.

In some embodiments, at least 85 wt. % of the lipid composition consists of octreotide chloride, glycerol dioleate, phosphatidylcholine, ethanol, and propylene glycol.

In some embodiments, at least 86 wt. % of the lipid composition consists of octreotide chloride, glycerol dioleate, phosphatidylcholine, ethanol, propylene glycol, EDTA, and the lipid composition has a water content of less than 1.0 wt. % at the release of the lipid composition product for sale.

In some embodiments, the lipid composition is administered by a syringe, a pre-filled syringe, an autoinjector, or a pen injector.

In some embodiments, the lipid composition is administered by subcutaneous injection.

In some embodiments, the lipid composition is administered not more than once every two weeks, and in a volume of about 1 mL.

In some embodiments, the patient has a maximum blood plasma concentration (Cₘₐₓ) of octreotide (at steady state) of less than 45 ng/ml, such as less than 40 ng/ml, less than 35 ng/ml, or less than 30 ng/ml, for each administration.

In some embodiments, the patient has a maximum blood plasma concentration (Cₘₐₓ) of octreotide (at steady state) of 3-45 ng/ml, such as 4-40 ng/ml, such as 5-35 ng/ml, for each administration.

In some embodiments, the patient has an average blood plasma concentration (C_{AV}) of octreotide (at steady state) of 3-24 ng/ml, such as 4-20 ng/ml, such as 5-15 ng/ml or 5-10 ng/ml, for each administration.

In some embodiments, the patient has an average blood plasma concentration (C_{AV}) of octreotide (at steady state) of 4-20 ng/ml for each administration.

In some embodiments, the patient has a plasma concentration AUC of octreotide (at steady state) of 1300-6700 ng*h/ml, such as 1700-5000 ng*h/ml or 1700-3400 ng*h/ml, for each administration.

In some embodiments, the patient has a plasma concentration AUC of octreotide (at steady state) of 1550-3500 ng*h/ml, such as 1600-3450 ng*h/ml, such as 1600-3400 ng*h/ml, for each administration.

In some embodiments, administering the lipid composition comprising 20 mg of octreotide provides a drug depot comprising octreotide in the patient, said depot providing a release of octreotide to the patient over about two weeks and wherein the patient has blood plasma levels of octreotide of at least about 3 ng/ml, such as at least about 4 ng/ml, about 5 ng/ml, or about 6 ng/ml, for each administration.

In some embodiments, the depot is in the subcutaneous tissue of the patient.

In some embodiments, the lipid composition is administered to the patient once every two weeks until the patient experiences progressive disease (PD), determined radiologically according to Response Evaluation Criteria in Solid Tumors version 1.1 (RECIST 1.1), whereupon the lipid composition is optionally administered to the patient once every week.

In some embodiments, the method provides a progression-free survival of at least 16 months, such as at least 17 months, such as at least 18 months, such as at least 19 months, such as at least 20 months, such as at least 21 months, such as at least 22 months, such as at least 23 months.

In some embodiments, the method provides an improved progression-free survival (PFS) compared to Sandostatin® LAR®.

In some embodiments, the method provides an improved progression-free survival (PFS) compared to placebo or Sandostatin® LAR®, and/or increased or about the same progression-free survival as Somatuline® Depot (Somatuline® Autogel), wherein the progression-free survival is determined radiologically according to Response Evaluation Criteria in Solid Tumors version 1.1 (RECIST 1.1).

In some embodiments, the method provides an improved overall response rate (ORR) compared to Sandostatin® LAR® as determined according to Response Evaluation Criteria in Solid Tumors version 1.1 (RECIST 1.1).

In some embodiments, the overall response rate is selected from the group consisting of complete response and/or partial response.

In some embodiments, the method provides an improved disease control rate (DCR) compared to Sandostatin® LAR® as determined according to Response Evaluation Criteria in Solid Tumors version 1.1 (RECIST 1.1).

In some embodiments, the patient has a maximum blood plasma concentration (Cₘₐₓ) of octreotide (at steady state) of less than 45 ng/ml, such as less than 40 ng/ml, less than 35 ng/ml, or less than 30 ng/ml, for each once every administration.

In some embodiments, the patient has a maximum blood plasma concentration (Cₘₐₓ) of octreotide (at steady state) of 3-45 ng/ml, such as 4-40 ng/ml, such as 5-35 ng/ml, for each once every administration.

In some embodiments, the patient has an average blood plasma concentration (C_{AV}) of octreotide (at steady state) of 6-40 ng/ml, such as 7-30 ng/ml or 7-25 ng/ml, for each once every week administration.

In some embodiments, the patient has an average blood plasma concentration (C_{AV}) of octreotide (at steady state) of 4-20 ng/ml for each once every week administration.

In some embodiments, the patient has a plasma concentration AUC of octreotide (at steady state) of 1000-6700 ng*h/ml, such as 1000-5900 ng*h/ml, such a 1200-5000 ng*h/ml, or 1200-4200 ng*h/ml, for each once every week administration.

In some embodiments, the patient has a plasma concentration AUC of octreotide (at steady state) of 1550-3500 ng*h/ml, such as 1600-3450 ng*h/ml, such as 1600-3400 ng*h/ml, for each once every week administration.

In some embodiments, administering the lipid composition comprising 20 mg of octreotide provides a drug depot comprising octreotide in the patient, said depot providing a release of octreotide to the patient over about 1 week and wherein the patient has blood plasma levels of octreotide of at least about 5 ng/ml, such as at least about 7 ng/ml, about 8 ng/ml, or about 10 ng/ml, for each once every week administration.

In some embodiments, disclosed herein is a pre-filled syringe comprising the aforementioned lipid composition and/or any of its disclosed variations.

In some embodiments, disclosed herein is an autoinjector comprising a glass compartment containing the aforementioned lipid composition and/or any of its disclosed variations.

In some embodiments, the glass compartment of the autoinjector is part of a pre-filled syringe.

In some embodiments, disclosed herein is a kit for the administration of octreotide or a pharmaceutically acceptable salt thereof having one or more containers comprising 20 mg of octreotide or a pharmaceutically acceptable salt thereof, wherein the octreotide or a pharmaceutically acceptable salt in the one or more containers is administered according to the aforementioned method and/or any of its disclosed variations.

### ITEMIZED LIST OF EMBODIMENTS

E1. A method of treating at least one neuroendocrine tumor comprising, administering to a patient in need thereof a lipid composition comprising 20 mg of octreotide or a pharmaceutically acceptable salt thereof, wherein the lipid composition is administered to the patient once every two weeks.

E2. The method of E1, wherein the lipid composition is administered as a unit dose.

E3. The method of E1 or E2, wherein the at least one neuroendocrine tumor is a gastroenteropancreatic neuroendocrine tumor.

E4. The method of any one of E1-E3, wherein the octreotide or a salt thereof is octreotide chloride.

E5. The method of E4, wherein the octreotide chloride is the sole active agent in the lipid composition.

E6. The method of any one of E1-E5, wherein the lipid composition further comprises glycerol dioleate, phosphatidylcholine, and ethanol.

E7. The method of E6, wherein the lipid composition further comprises propylene glycol.

E8. The method of E6 or E7, wherein the lipid composition further comprises EDTA.

E9. The method of E8, wherein the lipid composition further comprises ethanolamine and/or diethanolamine.

E10. The method of any one of E1-E9, wherein at least 85 wt % of the lipid composition consists of octreotide chloride, glycerol dioleate, phosphatidylcholine, ethanol, and propylene glycol.

E11. The method of any one of E1-E10, wherein at least 86 wt % of the lipid composition consists of octreotide chloride, glycerol dioleate, phosphatidylcholine, ethanol, propylene glycol, EDTA, and the lipid composition has a water content of less than 1.0 wt % at the release of the product for sale.

E12. The method of any one of E1-E11, comprising administering the lipid composition by a syringe, a pre-filled syringe, an autoinjector, or a pen injector.

E12. The method of any one of E1-E12, administering the lipid composition by subcutaneous injection.

E14. The method of any one of E1-E13, comprising administering the lipid composition not more than once every two weeks, and in a volume of about 1 mL.

E15. The method of any one of E1-E14, wherein the patient has a maximum blood plasma concentration (Cₘₐₓ) of octreotide (at steady state) of less than 45 ng/ml, such as less than 40 ng/ml, less than 35 ng/ml, or less than 30 ng/ml, for each administration.

E16. The method of any one of E1-E15, wherein the patient has a maximum blood plasma concentration (Cₘₐₓ) of octreotide (at steady state) of 3-45 ng/ml, such as 4-40 ng/ml, such as 5-35 ng/ml, for each administration.

E17. The method of any one of E1-E16, wherein the patient has an average blood plasma concentration (C_{AV}) of octreotide (at steady state) of 3-24 ng/ml, such as 4-20 ng/ml, such as 5-15 ng/ml or 5-10 ng/ml, for each administration.

E18. The method of any one of E1-E17, wherein the patient has an average blood plasma concentration (C_{AV}) of octreotide (at steady state) of 4-20 ng/ml for each administration.

E19. The method of any one of E1-E18, wherein the patient has a plasma concentration AUC of octreotide (at steady state) of 1300-6700 ng*h/ml, such as 1700-5000 ng*h/ml or 1700-3400 ng*h/ml, for each administration.

E20. The method of any one of E1-E19, wherein the patient has a plasma concentration AUC of octreotide (at steady state) of 1550-3500 ng*h/ml, such as 1600-3450 ng*h/ml, such as 1600-3400 ng*h/ml, for each administration.

E21. The method of any one of E1-E20, wherein administering the lipid composition comprising 20 mg of octreotide provides a drug depot comprising octreotide in the patient, said depot providing a release of octreotide to the patient over about two weeks and wherein the patient has blood plasma levels of octreotide of at least about 3 ng/ml, such as at least about 4 ng/ml, about 5 ng/ml, or about 6 ng/ml, for each administration.

E22. The method of E21, wherein the depot is in the subcutaneous tissue of the patient.

E23. The method of any one of E1-E22, wherein the lipid composition is administered to the patient once every two weeks until the patient experiences progressive disease (PD), determined radiologically according to Response Evaluation Criteria in Solid Tumors version 1.1 (RECIST 1.1), whereupon the lipid composition is optionally administered to the patient once every week.

E24. The method of any one of E1-E23, wherein the method provides a progression-free survival of at least 16 months, such as at least 17 months, such as at least 18 months, such as at least 19 months, such as at least 20 months, such as at least 21 months, such as at least 22 months, such as at least 23 months.

E25. The method of any one of E1-E24, wherein the method provides an improved progression-free survival (PFS) compared to Sandostatin® LAR®.

E26. The method of any one of E1-E25, wherein the method provides an improved progression-free survival (PFS) compared to placebo or Sandostatin® LAR®, and/or increased or about the same progression-free survival as Somatuline® Depot (Somatuline® Autogel), wherein the progression-free survival is determined radiologically according to Response Evaluation Criteria in Solid Tumors version 1.1 (RECIST 1.1).

E27. The method of any one of E1-E26, wherein the method provides an improved overall response rate (ORR) compared to Sandostatin® LAR® and/or improved or about the same ORR as Somatuline® Depot (Somatuline® Autogel) as determined according to Response Evaluation Criteria in Solid Tumors version 1.1 (RECIST 1.1).

E28. The method of E27, wherein the overall response rate is selected from the group consisting of complete response and/or partial response.

E29. The method of any one of E1-E28, wherein the method provides an improved disease control rate (DCR) compared to Sandostatin® LAR® and/or improved or about the same DCR as Somatuline® Depot (Somatuline® Autogel) as determined according to Response Evaluation Criteria in Solid Tumors version 1.1 (RECIST 1.1).

E30. The method of any one of E23-E29, wherein the patient has a maximum blood plasma concentration (Cₘₐₓ) of octreotide (at steady state) of less than 45 ng/ml, such as less than 40 ng/ml, less than 35 ng/ml, or less than 30 ng/ml, for each once every administration.

E31. The method of any one of E23-E30, wherein the patient has a maximum blood plasma concentration (Cₘₐₓ) of octreotide (at steady state) of 3-45 ng/ml, such as 4-40 ng/ml, such as 5-35 ng/ml, for each once every administration.

E32. The method of any one of E23-E31, wherein the patient has an average blood plasma concentration (C_{AV}) of octreotide (at steady state) of 6-40 ng/ml, such as 7-30 ng/ml or 7-25 ng/ml, for each once every week administration.

E33. The method of any one of E23-E32, wherein the patient has an average blood plasma concentration (C_{AV}) of octreotide (at steady state) of 4-20 ng/ml for each once every week administration.

E34. The method of any one of E23-E33, wherein the patient has a plasma concentration AUC of octreotide (at steady state) of 1000-6700 ng*h/ml, such as 1000-5900 ng*h/ml, such a 1200-5000 ng*h/ml, or 1200-4200 ng*h/ml, for each once every week administration.

E35. The method of any one of E23-E34, wherein the patient has a plasma concentration AUC of octreotide (at steady state) of 1550-3500 ng*h/ml, such as 1600-3450 ng*h/ml, such as 1600-3400 ng*h/ml, for each once every week administration.

E36. The method of any one of E23-E35, wherein administering the lipid composition comprising 20 mg of octreotide provides a drug depot comprising octreotide in the patient, said depot providing a release of octreotide to the patient over about 1 week and wherein the patient has blood plasma levels of octreotide of at least about 5 ng/ml, such as at least about 7 ng/ml, about 8 ng/ml, or about 10 ng/ml, for each once every week administration.

E37. A lipid composition comprising 20 mg of octreotide or a pharmaceutically acceptable salt thereof for use in treating at least one neuroendocrine tumor, and wherein the composition is administered to a patient once every two weeks.

E38. The composition of E37, wherein the at least one neuroendocrine tumor is a gastroenteropancreatic neuroendocrine tumor.

E39. The composition of E37 or E38, wherein the octreotide or a salt thereof is octreotide chloride.

E40. The composition of E39, wherein the octreotide chloride is the sole active agent in the lipid composition.

E41. The composition of any one of E37-E40, wherein the lipid composition further comprises glycerol dioleate, phosphatidylcholine, and ethanol.

E42. The composition of E41, wherein the lipid composition further comprises propylene glycol.

E43. The composition of any one of E37-E42, wherein the lipid composition further comprises EDTA.

E44. The composition of any one of E37-E43, wherein the lipid composition further comprises ethanolamine and/or diethanolamine.

E45. The composition of any one of E37-E44, wherein at least 85 wt % of the lipid composition consists of octreotide chloride, glycerol dioleate, phosphatidylcholine, ethanol, and propylene glycol.

E46. The composition of any one of E37-E45, wherein at least 86 wt % of the lipid composition consists of octreotide chloride, glycerol dioleate, phosphatidylcholine, ethanol, propylene glycol, EDTA, and the lipid composition has a water content of less than 1.0 wt % at the release of the product for sale.

E47. The composition of any one of E37-E46, comprising administering the lipid composition by a syringe, a pre-filled syringe, an autoinjector, or a pen injector.

E48. The composition of any one of E37-E47, administering the lipid composition by subcutaneous injection.

E49. The composition of any one of E37-E48, comprising administering the lipid composition not more than once every two weeks, and in a volume of about 1 mL.

E50. The composition of any one of E37-E49, wherein the patient has a maximum blood plasma concentration (Cₘₐₓ) of octreotide (at steady state) of less than 45 ng/ml, such as less than 40 ng/ml, less than 35 ng/ml, or less than 30 ng/ml, for each administration.

E51. The composition of any one of E37-E50, wherein the patient has a maximum blood plasma concentration (Cₘₐₓ) of octreotide (at steady state) of 3-45 ng/ml, such as 4-40 ng/ml, such as 5-35 ng/ml, for each administration.

E52. The composition of any one of E37-E51, wherein the patient has an average blood plasma concentration (C_{AV}) of octreotide (at steady state) of 4-20 ng/ml, such as 5-15 ng/ml or 5-10 ng/ml, for each administration.

E53. The composition of any one of E37-E52, wherein the patient has an average blood plasma concentration (C_{AV}) of octreotide (at steady state) of 4-20 ng/ml for each administration.

E54. The composition of any one of E37-E53, wherein the patient has a plasma concentration AUC of octreotide (at steady state) of 1300-6700 ng*h/ml, such as 1700-5000 ng*h/ml or 1700-3400 ng*h/ml, for each administration.

E55. The composition of any one of E37-E54, wherein the patient has a plasma concentration AUC of octreotide (at steady state) of 1550-3500 ng*h/ml, such as 1600-3450 ng*h/ml, such as 1600-3400 ng*h/ml, for each administration.

E56. The composition of any one of E37-E55, wherein administering the lipid composition comprising 20 mg of octreotide provides a drug depot comprising octreotide in the patient, said depot providing a release of octreotide to the patient over about two weeks and wherein the patient has blood plasma levels of octreotide of at least about 3 ng/ml, such as at least about 4 ng/ml, about 5 ng/ml, or about 6 ng/ml, for each administration.

E57. The composition of E56, wherein the depot is in the subcutaneous tissue of the patient.

E58. The composition of any one of E37-E57, wherein the lipid composition is administered to the patient once every two weeks until the patient experiences progressive disease (PD), determined radiologically according to Response Evaluation Criteria in Solid Tumors version 1.1 (RECIST 1.1), whereupon the lipid composition is optionally administered to the patient once every week.

E59. The composition of any one of E37-E58, wherein the administration of the composition provides a progression-free survival of at least 16 months, such as at least 17 months, such as at least 18 months, such as at least 19 months, such as at least 20 months, such as at least 21 months, such as at least 22 months, such as at least 23 months.

E60. The composition of any one of E37-E59, wherein the method provides an improved progression-free survival (PFS) compared to Sandostatin® LAR®.

E61. The composition of any one of E37-E60, wherein the administration of the composition provides an improved progression-free survival (PFS) compared to placebo or Sandostatin® LAR®, and/or increased or about the same progression-free survival as Somatuline® Depot (Somatuline® Autogel), wherein the progression-free survival is determined radiologically according to Response Evaluation Criteria in Solid Tumors version 1.1 (RECIST 1.1).

E62. The composition of any one of E37-E61, wherein the administration of the composition provides an improved overall response rate (ORR) compared to Sandostatin® LAR® and/or improved or about the same ORR as Somatuline® Depot (Somatuline® Autogel) as determined according to Response Evaluation Criteria in Solid Tumors version 1.1 (RECIST 1.1).

E63. The composition of E62, wherein the overall response rate is selected from the group consisting of complete response and/or partial response.

E64. The composition of any one of E37-E53, wherein the administration of the composition provides an improved disease control rate (DCR) compared to Sandostatin® LAR® and/or improved or about the same DCR as Somatuline® Depot (Somatuline® Autogel) as determined according to Response Evaluation Criteria in Solid Tumors version 1.1 (RECIST 1.1).

E65. The composition of any one of E58-E64, wherein the patient has an average blood plasma concentration (C_{AV}) of octreotide (at steady state) of 6-40 ng/ml, such as 7-30 ng/ml or 7-25 ng/ml, for each once every week administration.

E66. The composition of any one of E58-E65, wherein the patient has an average blood plasma concentration (C_{AV}) of octreotide (at steady state) of 4-20 ng/ml for each once every week administration.

E67. The composition of any one of E58-E66, wherein the patient has a plasma concentration AUC of octreotide (at steady state) of 1000-6700 ng*h/ml, such as 1000-5900 ng*h/ml, such a 1200-5000 ng*h/ml, or 1200-4200 ng*h/ml, for each once every week administration.

E68. The composition of any one of E58-E67, wherein the patient has a plasma concentration AUC of octreotide (at steady state) of 1550-3500 ng*h/ml, such as 1600-3450 ng*h/ml, such as 1600-3400 ng*h/ml, for each once every week administration.

E69. The composition of any one of E58-E68, wherein administering the lipid composition comprising 20 mg of octreotide provides a drug depot comprising octreotide in the patient, said depot providing a release of octreotide to the patient over about 1 week and wherein the patient has blood plasma levels of octreotide of at least about 5 ng/ml, such as at least about 7 ng/ml, about 8 ng/ml, or about 10 ng/ml, for each once every week administration.

E70. A pre-filled syringe comprising a lipid composition according to any one of E37-E69.

E71. An autoinjector comprising a glass compartment containing the lipid composition of any one of E37-E69.

E72. The autoinjector of E71, wherein the compartment is part of a pre-filled syringe.

E73. A kit for the administration of octreotide or a pharmaceutically acceptable salt thereof having one or more containers comprising 20 mg of octreotide or a pharmaceutically acceptable salt thereof, wherein the octreotide or a pharmaceutically acceptable salt in the one or more containers is administered according to the method of any one of E1-E36.

### Example 1 Clinical Trial Protocol Used Herein

### 1 Abbreviations and Terms

| | |
|---|---|
| ¹⁷⁷Lu | Lutetium 177 |
| ⁶⁸Ga | Gallium-68 |
| AE | Adverse event |
| ADR | Adverse drug reaction |
| ALT | Alanine aminotransferase |
| ASCO | American Society of Clinical Oncology |
| AST | Aspartate aminotransferase |
| ATC | Anatomical Therapeutic Chemical |
| ATG | Autogel |
| AUCₛₛ | Area under the plasma concentration-time curve |
| | at steady state |
| BIRC | Blinded Independent Review Committee |
| BMI | Body mass index |
| C_{av, ss} | Average plasma concentration during a dosing |
| | interval at steady state |
| CI | Confidence interval |
| Cₘₐₓ | Maximum plasma concentration |
| C_{max, ss} | Maximum plasma concentration at steady state |
| CR | Complete response |
| CRO | Contract research organization |
| CT | Computed tomography |
| CTCAE | Common Terminology Criteria for Adverse Events |
| CYP | Cytochrome P450 |
| DCR | Disease control rate |
| DMC | Data Monitoring Committee |
| ECG | Electrocardiogram |
| ECOG | Eastern Cooperative Oncology Group |
| eCRF | Electronic case report form |
| EORTC | European Organization for Research and Treatment |
| QLQ-C30 | of Cancer's Core Quality of Life Questionnaire |
| FDG | Fluorodeoxy glucose |
| GCP | Good Clinical Practice |
| GEP | Gastroenteropancreatic |
| GI | Gastrointestinal |
| HbA1c | Hemoglobin A1c |
| HCP | Healthcare professional |
| HIV | Human immunodeficiency virus |
| IB | Investigator's Brochure |
| ICF | Informed consent form |
| ICH | International Council for Harmonisation |
| IEC | Independent Ethics Committee |
| IM | Intramuscular |
| IMP | Investigational medicinal product |
| IR | Immediate release |
| IRB | Institutional Review Board |
| ITT | Intent-to-treat |
| Ki-67 | A nuclear antigen |
| LAR | Long-acting release |
| MedDRA | Medical Dictionary for Regulatory Activities |
| MMRM | Mixed model for repeated measures |
| MRI | Magnetic resonance imaging |
| mTOR | Mammalian target of rapamycin |
| NCI | National Cancer Institute |
| NEC | Neuroendocrine carcinomas |
| NET | Neuroendocrine tumors |
| NIMP | Non-investigational medicinal product |
| NOAEL | No-observed-adverse-effect-level |
| ORR | Overall response rate |
| PD | Progressive disease |
| PET | Positron emission tomography |
| PFS | Progression-free survival |
| PFS2 | Progression-free survival 2 |
| PFS-ext | Progression-free survival in the Extension |
| | Treatment Period |
| PGI-S | Patient Global Impression of Severity |
| PK | Pharmacokinetic(s) |
| PQC | Product Quality Complaint |
| PR | Partial response |
| PRO | Patient-reported outcome |
| PRRT | Peptide receptor radionuclide therapy |
| QLQ-GINET21 | Quality of Life Questionnaire - Neuroendocrine |
| | Carcinoid Module |
| QTcF | QTc interval corrected by Fridericia's formula |
| RECIST 1.1 | Response Evaluation Criteria in Solid |
| | Tumors version 1.1 |
| SAE | Serious adverse event |
| SAP | Statistical analysis plan |
| SC | Subcutaneous |
| SD | Stable disease |
| SF-36 | Short Form-36 |
| SSA | Somatostatin analogue |
| TEAE | Treatment-emergent adverse event |
| TSQM | Treatment Satisfaction Questionnaire for Medication |
| ULN | Upper limit of normal |
| US | United States |

### 2 Introduction

### 2.1 Background

### 2.1.1 Indication

Neuroendocrine tumors (NET) are malignant neoplasms that account for about 0.5% of all newly diagnosed malignancies. NET originate from neuroendocrine cells of embryonic origin and therefore, can arise in many anatomic sites, such as the gastroenteropancreatic (GEP) tract, the lungs, or the central nervous system (1). Despite certain common morphological and immunohistochemical features, there is significant heterogeneity in the prognoses and treatment strategies according to the primary site, histological differentiation (poorly or well-differentiated), and stage of the tumors. According to the 2019 version of the World Health Organization classification of tumors, neuroendocrine neoplasms are divided into NET and neuroendocrine carcinomas (NEC) based on their molecular differences (2). NET are further divided into grades according to their histopathologic characterization and to their proliferation (nuclear antigen Ki-67) index; see Table 1 above.

A review of Surveillance, Epidemiology and End Results data showed an incidence of 1.09 cases per 100,000 people per year in 1973 (3). The incidence rate of NET has increased to approximately 7 cases per 100,000 people per year in the United States (US) in 2012 (4). The majority (50-70%) of NET diagnosed in Western countries are GEP-NET (5). The GEP-NET may have the following primary tumor locations: stomach, duodenum, jejunum, ileum, pancreas, appendix, cecum, colon and rectum.

The diagnosis of GEP-NET is established pathologically. Patients are further characterized according to the clinical presentation, including whether symptoms related to tumor hormone secretion are present. Carcinoid syndrome is the most notable cluster of symptoms related to secretion of serotonin and other vasoactive peptide hormones. It is characterized by abdominal pain and cramping, severe diarrhea and flushing, and potential cardiac abnormalities. Carcinoid syndrome is more frequent in patients with advanced disease (6).

Radiology imaging plays an important role in the staging and characterization of somatostatin receptor expression in NET. Conventional cross-sectional imaging using computed tomography (CT) or magnetic resonance imaging (MRI) scans plays a key role in the assessment of location and extent of the disease.

Functional imaging studies for patients with NET are based primarily on tumor somatostatin receptor expression and were historically performed using indium-111 pentetreotide somatostatin-receptor scintigraphy. In recent years, somatostatin-receptor positron emission tomography (PET)/CT scanning, using modalities such as gallium-68 (⁶⁸Ga)-DOTATATE PET/CT has become the preferred modality for somatostatin-receptor imaging as result of its higher sensitivity, reduced radiation exposure, and improved convenience to patients (1-day versus multiday scanning).

GEP-NET have a variable prognosis based on primary site of disease, degree of differentiation, grade, expression of somatostatin receptors, and presence of metastases at diagnosis. Well-differentiated, lower-grade GEP-NET have prolonged survival compared to high-grade tumors, and pancreatic NET generally have more aggressive biology than NET arising in the small intestine. Retrospective analyses have demonstrated that the median overall survival for pancreatic NET ranges from 2 to 5.8 years, while NET in the small intestine have a median overall survival of almost 8 years (4, 7, 8).

Surgical resection of the primary lesions remains the mainstay of treatment for NET and the only way to obtain a cure. However, resection is often not possible as NET are frequently detected in a more advanced tumor stage when metastases have already developed (1). In patients with GEP-NET and disease progression, somatostatin analogues (SSAs), mammalian target of rapamycin (mTOR) inhibitors, tyrosine kinase inhibitors (sunitinib), alkylating agents, and peptide receptor radionuclide therapy (PRRT) provide treatment options (9-13).

Currently, the standard of care for initial treatment of unresectable or metastatic, low-grade GEP-NET according to the National Cancer Comprehensive Network is to initiate treatment with SSAs, such as octreotide or lanreotide. Clinical trials have shown that octreotide long-acting release (LAR) (Sandostatin® LAR®) and lanreotide autogel (ATG) (Somatuline® Depot or Somatuline® Autogel®) increase time to tumor progression or progression-free survival (PFS) when compared to placebo in treatment-naïve patients with advanced NET. In a trial in patients with gastrointestinal (GI) NET (the PROMID trial), patients who received octreotide LAR 30 mg/month had more than double the time to tumor progression (14.3 months) compared with patients who received placebo (6.0 months); p=0.000072. Overall, 67% of the 42 patients treated with octreotide LAR achieved stable disease (SD) compared with 37% of the 43 patients who received placebo (14). In another trial (the CLARINET trial) patients with GEP-NET treated with lanreotide ATG had significantly prolonged PFS when compared to placebo (median PFS not reached versus 18.0 months, p<0.001; hazard ratio for disease progression or death with lanreotide ATG versus placebo, 0.47; 95% confidence interval [CI]: 0.30 to 0.73) (15). Furthermore, in atrial of 231 patients (the NETTER-1 trial), in which patients with well-differentiated GEP-NET (N=117) were treated with lutetium 177 (¹⁷⁷Lu)-Dotatate plus octreotide LAR or with octreotide LAR alone (control), median PFS was not reached in the ¹⁷⁷Lu-Dotatate group, whereas it was 8.4 months (95% CI: 5.8 to 9.1) in the control group (N=114) (hazard ratio for disease progression or death with ¹⁷⁷Lu-Dotatate versus control, 0.21; 95% CI: 0.13 to 0.33; p<0.001) (16).

Where approved, the dose of octreotide LAR for the treatment of advanced midgut NET is 30 mg/month, which is also the maximum approved dose for symptom control. Higher doses of octreotide up to 120 mg have been used for symptom control in patients who no longer respond adequately to standard doses. Higher doses have also been used for tumor control, though results from controlled studies are lacking (17). Three studies have investigated the antiproliferative efficacy of above-standard doses of either octreotide LAR (up to 160 mg every 2 weeks) or lanreotide (up to 15 mg/day), showing disease stabilization in 37 to 75% of the patients (18-20). In a recently completed trial, the CLARINET FORTE trial (21, 22), the efficacy of lanreotide ATG 120 mg administered every 2 weeks was investigated in patients with Grade 1 or Grade 2, metastatic or locally advanced, unresectable pancreatic or midgut NET after they had progressed on the standard dose of lanreotide ATG (120 mg every 4 weeks). The median PFS (primary endpoint in the trial) was 5.6 months (95% CI: 5.5 to 8.3) in the pancreatic NET cohort (N=48) and 8.3 months (95% CI: 5.6 to 11.1) in the midgut NET cohort (N=51). Another trial, NETTER-2 (23), is investigating if ¹⁷⁷Lu-Dotatate combined with octreotide LAR prolongs PFS in patients with GEP-NET and high-proliferation rate tumors (Grade 2 and Grade 3), when given as a first-line treatment compared to treatment with high-dose (60 mg) octreotide LAR given every 4 weeks. Furthermore, retrospective studies suggest that high doses of octreotide may improve overall survival and delay the time to other types of intervention (24, 25). In a small (N=28), sequential study, increased frequency of dosing of SSAs resulted in delayed time to tumor progression and time to biochemical progression (26). Recent reviews of the literature also suggest that increasing the dose or the dosing frequency of SSAs may provide additional therapeutic effect in treatment of progressive NET (27, 28). However, due to the nature of these studies (retrospective, limited number of patients, short follow-up) no definitive conclusions can be made. Prospective trials are needed to more definitively assess the efficacy of high-dose SSA therapy compared to standard-dose treatment.

### 2.1.2 CAM2029

CAM2029 (‘octreotide subcutaneous depot’) is a novel and long-acting pharmaceutical formulation of octreotide for subcutaneous (SC) administration. Compared to Sandostatin LAR, which is administered as an intramuscular (IM) injection and which needs to be reconstituted before injection, CAM2029 is provided in a pre-filled syringe with no need for reconstitution. CAM2029 also offers the option of self- or partner-administration and may be easier to handle and to administer, thereby potentially improving patient convenience and care. In addition, the bioavailability of octreotide has been shown to be higher for CAM2029 than for Sandostatin LAR (see below).

The non-clinical development program for CAM2029 includes bridging studies of toxicity, pharmacokinetics (PK) and local tolerability. In addition, the FluidCrystal® vehicle and the excipient glycerol dioleate have been evaluated. The main effects observed in these studies were reversible injection-site reactions, which were noted with the FluidCrystal vehicle, CAM2029 formulations, and glycerol dioleate. Body weight decreases were also observed, which is consistent with the pharmacology of octreotide. The maximum plasma concentration (Cₘₐₓ) of octreotide after administration of CAM2029 in dogs was higher than after administration of an equivalent dose of Sandostatin LAR, without any apparent difference in toxicity. The safety of CAM2029 is supported by the available extensive database (pharmacology, PK, safety, and toxicity) of octreotide (see further details in the Investigator's Brochure [IB]) (29). The scope of the available safety data of octreotide, the non-clinical studies conducted with CAM2029, and the literature addressing the reproductive and carcinogenic potential for the excipients support the current Phase 3 trial.

The clinical program for CAM2029 includes 3 completed Phase 1 trials in healthy volunteers (a single-dose trial [HS-05-194] and 2 repeated-dose trials [HS-07-291 and HS-11-411]), and 1 completed, repeated-dose, Phase 2 trial (HS-12-455) in patients with acromegaly or functioning NET previously treated with Sandostatin LAR. In addition, CAM2029 is currently being studied in US and Europe in 2 multinational Phase 3 trials in patients with acromegaly. A Phase 1 trial is also ongoing, in which the PK of octreotide after administration of CAM2029 with an autoinjector is investigated.

The results of the completed Phase 1 clinical trials showed that octreotide release from CAM2029 had a rapid onset of action, with an octreotide Cₘₐₓ observed within approximately 4 to 24 hours after dosing. Thereafter, the plasma concentration slowly declined over time with therapeutic drug levels maintained for approximately 4 weeks, resulting in observable suppression of insulin-like growth factor-1 over the same period. Dose-proportional PK was observed in the dose range of 10 to 30 mg CAM2029. The bioavailability of octreotide was approximately 5 times higher for CAM2029 than for Sandostatin LAR (trial HS-11-411).

The Phase 2 trial showed that switching from Sandostatin LAR (10 mg, 20 mg, or 30 mg) to CAM2029 (20 mg once monthly or 10 mg every 2 weeks) was associated with maintenance or decrease in insulin-like growth factor-1 levels (as compared to pre-switch values) and in maintenance of growth hormone levels in patients with acromegaly, and maintenance or improvement of symptom control, as measured by flushing episodes and bowel movements, in patients with NET. The PK data from the trial confirmed the results from the trials in healthy volunteers and showed a higher exposure of octreotide after treatment with CAM2029 than after treatment with Sandostatin LAR.

Safety, including local injection-site tolerability, was investigated in all trials. The adverse event (AE) profile seen with CAM2029 was consistent with what has been recorded for octreotide LAR and octreotide immediate release (IR), with transient and mild to moderate GI events being the most frequently reported AEs.

### 2.2 Rationale for Conducting the Trial

The target population in the current trial comprises adult patients with histologically confirmed, advanced (unresectable and/or metastatic), and well-differentiated NET of presumed GEP origin. SSAs (octreotide and lanreotide) are often used as first-line therapy in patients with advanced GEP-NET. The current trial aims to confirm efficacy and safety of CAM2029 (octreotide SC depot) as compared to Investigator's choice of comparator (octreotide LAR or lanreotide ATG) for the treatment of patients with well-differentiated GEP-NET. As described above, retrospective and non-randomized studies suggest that treatment with high doses of SSAs can provide improved disease control compared to treatment with standard doses of SSAs. Previous trials with CAM2029 have shown that treatment with CAM2029 can achieve higher plasma levels of octreotide than standard doses of octreotide LAR. The patients in the trial will be treated with CAM2029 every 2 weeks or the comparator of choice (octreotide LAR or lanreotide ATG) every 4 weeks until disease progression. Patients in both treatment groups then have the option to switch to treatment with CAM2029 once weekly, to investigate potential further treatment benefits of a higher exposure of octreotide.

### 2.3 Benefit/Risk Assessment

### 2.3.1 Benefit Assessment

Octreotide products (Sandostatin IR and Sandostatin LAR) have well-characterized profiles of efficacy and safety with over 30 years of clinical use. CAM2029 has the same active ingredient (octreotide) as Sandostatin IR and Sandostatin LAR and has been shown to have a safety profile similar to Sandostatin LAR in previous Phase 1 and 2 clinical trials (29). Lanreotide is another SSA approved for the treatment of acromegaly and NET and is available in a microsphere formulation (sustained release) and a saturated aqueous solution (lanreotide ATG; Somatuline Autogel [Somatuline Depot in the US]). Lanreotide has been on the market for almost 20 years and its efficacy and safety are well established.

In the current trial, patients will be randomized to 1 of 2 treatment groups: CAM2029 20 mg every 2 weeks or one of the comparator products (30 mg octreotide LAR or 120 mg lanreotide ATG) every 4 weeks. Patients will be treated with the investigational medicinal products (IMP; i.e. CAM2029, octreotide LAR or lanreotide ATG) until disease progression, at which time patients in both the CAM2029 treatment group and the comparator treatment group will have the option to switch to treatment with CAM2029 20 mg once weekly. The shorter dosing interval could potentially result in a higher somatostatin-receptor saturation and may further improve the clinical outcomes.

The procedure for preparation of octreotide LAR suspension for injection encompasses several steps, including very gentle handling to ensure homogenous suspension of the product. Because of the IM injection and the complex preparatory handling procedure, administration of octreotide LAR needs to be performed by a trained healthcare professional (HCP). The provision of CAM2029 as a ready-to-use, long-acting formulation that is administered as a small-volume SC injection in a pre-filled syringe (i.e. eliminating the need for reconstitution) with a thin needle and with higher bioavailability of octreotide than for octreotide LAR, is expected to lead to improved quality of patient care and treatment convenience.

During the trial, self- or partner-administration of CAM2029 in the abdomen or thigh is allowed after appropriate training under the supervision of trained trial personnel and after the patient or their partner has been judged capable to administer CAM2029. A “partner” may be a spouse, parent, child, or sibling, or any other person whom the patient trusts to administer the injection. Home administration of CAM2029 is also allowed in the trial, which will further facilitate the treatment with CAM2029.

### 2.3.2 Risk Assessment

The CAM2029 drug product is based on the FluidCrystal injection depot technology. CAM2029 and other products based on this technology have been extensively investigated in Phase 1 to Phase 3 clinical trials. The buprenorphine-containing product Buvidal®, which is based on the FluidCrystal injection depot technology, was approved by the European and Australian authorities in November 2018 for treatment of opioid dependence. All clinical trials reported to date have demonstrated good or very good local tolerability, with generally mild or moderate transient injection-site reactions, such as injection site pain, pruritus, erythema, swelling and induration, at low incidence. The safety profile of Buvidal is also favorable based on post-marketing experience with more than 7,200 patient-years of exposure as of 30 Nov. 2020.

The active substance in CAM2029 (octreotide) is well documented. Octreotide products have been in clinical use for more than 30 years. The most commonly reported adverse drug reactions (ADRs) in clinical trials with octreotide products have been GI disorders (such as nausea, diarrhea, abdominal pain, flatulence and constipation), headache, cholelithiasis, hyperglycemia and injection-site reactions (30). The AE profile of CAM2029 in the clinical trials performed so far has been consistent with the AE profile of Sandostatin IR and Sandostatin LAR. In the clinical trials with CAM2029, the most commonly reported ADRs were headache and GI disorders, such as diarrhea and abdominal pain. Injection site reactions (e.g. injection-site pain, erythema, swelling, and pruritus) were also observed. The local injection-site reactions were generally of mild or moderate intensity. Only one serious adverse event (SAE) possibly related to CAM2029 has been recorded in the 4 clinical trials performed to date (circulatory collapse in a healthy volunteer, due to diarrhea and vomiting).

It is of note that even though the previous clinical trials showed that the bioavailability of octreotide was approximately 5 times higher for CAM2029 than for Sandostatin LAR, there were no significant differences between these drugs in their safety profiles (occurrence or severity of AEs). Furthermore, a retrospective literature analysis has indicated that increasing the dose intensity or the dose frequency of SSAs was well tolerated overall (27), suggesting that shortening the dose interval of CAM2029 20 mg to once-weekly dosing in the optional part of the trial for patients who experienced disease progression on less frequent dosing of CAM2029 or on comparator product is not expected to constitute an increased risk for the patients.

The patients will be followed closely with several safety assessments throughout the trial. Parameters that will be monitored regularly include vital signs, hematology laboratory assessments, blood chemistry (including renal and liver function, and thyroid hormones), urinalysis, electrocardiogram (ECG), and gallbladder ultrasound. Patients will be regularly and carefully monitored for AEs, including those that have been observed for Sandostatin IR and Sandostatin LAR. A Data Monitoring Committee (DMC) will be established and the DMC will conduct periodic reviews of safety data from the trial. In addition, the protocol provides specific guidance for IMP discontinuation and safety follow-up for ADRs, liver toxicity (increased liver enzyme values) and QT prolongation. Furthermore, sampling for measurement of octreotide plasma concentrations will be conducted in patients who receive CAM2029 or octreotide LAR. Blood samples will also be taken for qualification and quantification of anti-octreotide antibodies to assess for potential immunogenicity.

The Sponsor will, in collaboration with the contract research organization (CRO) managing the trial, prepare a specific COVID-19-related risk assessment and mitigation plan to be followed during the trial.

### 2.3.3 Overall Benefit/Risk Conclusions

CAM2029 has been found to be well tolerated in clinical trials conducted to date. The overall results from the clinical trials performed with CAM2029, together with the data from the non-clinical safety, toxicology, and PK studies, support a favorable benefit-risk profile of CAM2029 for further clinical development. Based on the available information, it is considered that the benefits outweigh the risks in the trial patient population.

### 3 Trial Objectives and Endpoints

The objectives of this trial and the corresponding endpoints are listed in Table 2.

TABLE 2
Trial objectives and endpoints

| Primary Objectives | Primary Endpoints |
|---|---|
| To assess superiority of treatment | PFS, defined as the time from |
| with CAM2029 compared to treatment | the date of randomization to |
| with octreotide LAR or lanreotide | the date of the first documented |
| ATG on PFS in patients with | disease progression as per |
| unresectable/metastatic and well- | RECIST 1.1 or death due to |
| differentiated GEP-NET | any cause, whichever occurs |
| | first, as assessed by a BIRC |

| Secondary Objectives | Secondary Endpoints |
|---|---|
| To assess superiority of treatment | PFS using RECIST 1.1 as assessed by local |
| with CAM2029 compared to treatment with | Investigators |
| octreotide LAR or lanreotide ATG with respect | |
| to PFS based on local Investigator assessment | |
| To compare the 2 treatment groups | Overall survival |
| with respect to overall survival | |
| To evaluate the 2 treatment groups | ORR, defined as the proportion of patients with best |
| with respect to ORR and DCR | overall response of CR or PR as per RECIST 1.1. |
| | DCR, defined as the proportion of patients with best |
| | overall response of CR, PR or SD as per RECIST |
| | 1.1 |
| To describe time to tumor response and duration of | Time to response and duration of response as per |
| response in the 2 treatment groups | RECIST 1.1 |
| To evaluate the need for rescue medication for | Average number of injections of octreotide rescue |
| symptom control in the 2 treatment groups | medication per month for each patient during the |
| | trial |
| | Total dosage and dose intensity of rescue medication |
| To assess the PK of octreotide after CAM2029 | Octreotide plasma concentrations over time |
| administration | |
| To assess octreotide exposure-response | Correlation between octreotide concentration and |
| relationship for CAM2029 | other endpoints or measures as appropriate |
| To assess supervised self- or partner-administration | Proportion of patients/partners declared competent |
| of CAM2029 | by trial personnel to administer CAM2029 out of |
| | those trying |
| To evaluate PROs for health-related quality | Change from baseline in QLQ-GINET21, SF-36, |
| of life in the 2 treatment groups | and the global health status/quality of life |
| | scale score of the EORTC QLQ-C30 |
| To evaluate the 2 treatment groups with respect | TSQM scores over time using all 4 domains of |
| to patient satisfaction with the treatment | TSQM (effectiveness, side effects, convenience, |
| | and global satisfaction) |
| To confirm the safety and tolerability of CAM2029 | AEs (including local tolerability) |
| in patients with unresectable/metastatic and well- | Changes in laboratory values, vital signs, ECG |
| differentiated GEP-NET | readings and gallbladder imaging |

| Exploratory Objectives | Exploratory Endpoints |
|---|---|
| To describe the effect of intensified | PFS-ext, defined as time from date of |
| treatment with CAM2029 in the Open-label | randomization to the date of documented |
| Extension Treatment Period | disease progression as per RECIST 1.1 or |
| | death from any cause, whichever occurs |
| | first, in the Open-label Extension Treatment |
| | Period, as assessed by a BIRC |
| To compare the 2 treatment groups with | PFS2, defined as time from date of |
| respect to PFS2 based on local | randomization to the date of documented |
| Investigator assessment | progression as per RECIST 1.1 on next-line |
| | therapy or death from any cause, whichever |
| | occurs first |
| To assess hospital resource utilization | Number of patients hospitalized |
| | Total number and length of hospitalizations |
| To evaluate the 2 treatment groups | Describe patients' experiences with |
| with respect to treatment experience | the trial regimen and trial activities using |
| | data from patient exit interviews |
| | Explore the minimal important difference for |
| | EORTC QLQ-C30 using patient exit interviews |
| To evaluate the 2 treatment groups | To explore the minimal important difference |
| with respect to patients' impression | for EORTC QLQ-C30 using the PGI-S and patient |
| of disease severity | exit interviews |
| To evaluate the 2 treatment groups with | Time to deterioration of ECOG performance |
| respect to ECOG performance status | status |
| To assess the immunogenicity of octreotide in | Qualification and quantification of anti- |
| patients with metastatic/inoperable and well- | octreotide antibodies |
| differentiated GEP-NET | |

AE: adverse event;
ATG: autogel;
BIRC: Blinded Independent Review Committee;
CR: complete response;
DCR: disease control rate;
ECG: electrocardiogram;
ECOG: Eastern Cooperative Oncology Group;
EORTC QLQ-C30: European Organization for Research and Treatment of Cancer's Core Quality of Life Questionnaire;
GEP-NET: gastroenteropancreatic neuroendocrine tumors;
LAR: long-acting release;
ORR: overall response rate;
PFS: progression-free survival;
PFS2: progression-free survival 2;
PFS-ext: progression-free survival in the Extension Treatment Period;
PGI-S: Patient Global Impression of Severity;
PK: pharmacokinetics;
PR: partial response;
PROs: patient-reported outcomes;
QLQ-GINET21: Quality of Life Questionnaire - Neuroendocrine Carcinoid Module;
RECIST 1.1: Response Evaluation Criteria in Solid Tumors version 1.1;
SD: stable disease;
SF-36: Short Form-36;
TSQM: Treatment Satisfaction Questionnaire for Medication

### 4 Investigational Plan

### 4.1 Overall Trial Design and Plan

This is a prospective, multi-center, randomized, open-label, parallel-group, Phase 3 trial comparing the efficacy of treatment with CAM2029 20 mg every 2 weeks to treatment with the Investigator's choice of comparator, i.e. octreotide LAR 30 mg or lanreotide ATG 120 mg every 4 weeks (the comparator treatment group) in patients with advanced, well-differentiated GEP-NET. Approximately 300 patients will be randomized to 1 of 2 treatment groups in the Open-label Randomized Treatment Period. The patients will be followed until disease progression and the primary PFS analysis will be performed after 194 events (i.e. disease progression) during the Randomlzed Treatment Period.

Patients who experience progressive disease (PD) during the Randomized Treatment Period may enter an optional Open-label Extension Treatment Period, during which they will be treated with CAM2029 20 mg once weekly.

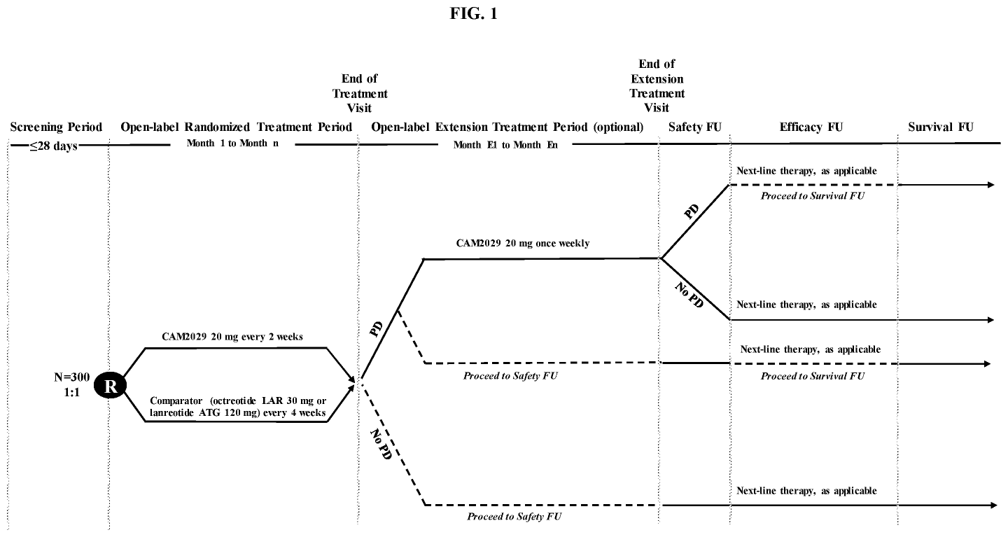

An overview of the trial design is shown in FIG. 1.

After the primary PFS analysis (based on the Blinded Independent Review Committee [BIRC] assessment of PD), patients with PD will be followed for up to 2 years. Patients who are still ongoing in the comparator treatment group at the time of the primary PFS analysis will have the possibility to be switched to receive treatment with CAM2029 20 mg every 2 weeks, if the trial meets the primary objective.

### 4.1.1 Screening Period

At screening, patients must provide written informed consent to participate in the trial before any trial-related procedures are performed. The patient's eligibility will be confirmed, and demographics, medical history (including GEP-NET medical and treatment history), and prior medications will be recorded. Blood and urine samples will be collected for assessment of clinical laboratory parameters (hematology, biochemistry, urinalysis, thyroid hormones, and serology) and a serum pregnancy test will be performed (if applicable). A complete physical examination will be performed and vital signs, height, weight, body mass index (BMI), and ECG will be recorded. A gallbladder ultrasound will also be performed. Patients will undergo a CT or MRI scan of the chest, pelvis, and abdomen. Presence of somatostatin receptors on the lesions will also be confirmed. For patients with Grade 3 NET, it is strongly encouraged to perform a fluorodeoxyglucose (FDG)-PET to delineate heterogenous disease and to rule out more aggressive FDG-avid disease that is not somatostatin-receptor positive and not well suited to SSA treatment. The Screening Period will have a duration of up to 28 days.

### 4.1.2 Open-Label Randomized Treatment Period

Eligible patients will be randomized in a 1:1 ratio to 1 of the 2 treatment groups on Day 1:
CAM2029 treatment group (CAM2029 20 mg, administered every 2 weeks)
Comparator treatment group (Investigator's choice of octreotide LAR 30 mg or lanreotide ATG 120 mg, administered every 4 weeks)

Randomization will be stratified by the following:
Histological grade Ki-67<10% versus Ki-67≥10%
Tumor origin (pancreas versus other GI origin)
Intended choice of comparator (octreotide LAR or lanreotide ATG)

During the Randomized Treatment Period, tumor progression will be evaluated every 12 weeks. Safety will be evaluated continuously and plasma samples for assessment of octreotide concentration will be taken in patients who receive CAM2029 or octreotide LAR. Patient-reported treatment satisfaction will be assessed using a general questionnaire and will be compared between CAM2029 and the comparator products. Health-related quality of life parameters will also be assessed by patient-reported outcomes (PROs).

Patients randomized to the CAM2029 treatment group may self-administer CAM2029 or have CAM2029 administered by their partner. The feasibility of self- or partner-administration of CAM2029 under the supervision of a trained trial personnel will be assessed. If the trial personnel consider the patients or their partners competent to administer CAM2029, they may continue to administer CAM2029 every 2 weeks. If CAM2029 is not self- or partner-administered, trial personnel will perform the administrations at the clinic. Self- or partner-administration at home is allowed after 3 supervised and successful administrations.

Patients will continue to receive the randomized IMP treatment (CAM2029/comparator) as scheduled, until they experience any of the following:
Disease progression as confirmed by the BIRC
Unacceptable toxicity that precludes further treatment
Discontinuation of treatment at the discretion of the Investigator or patient
Lost to follow-up
Death

All patients who have PD (radiologically documented according to Response Evaluation Criteria in Solid Tumors version 1.1 [RECIST 1.1]) determined by the local Investigator will require an expedited tumor response review by the BIRC. In cases of discordance (i.e. the BIRC does not confirm the Investigator's assessment of PD), the patient should continue with IMP treatment (if clinically acceptable) until progression has been determined by the BIRC.

If the patient discontinues the randomized IMP treatment, the patient will be asked to return for an End-of-treatment Visit (see further details in Section [000280]).

### 4.1.3 End-of-Treatment Visit

Patients who discontinue IMP treatment during the Randomized Treatment Period should be scheduled for an End-of-treatment Visit within 28 days after the date the IMP treatment is discontinued, at which time all of the assessments listed for the End-of-treatment Visit in Table 5 will be performed. If the decision to discontinue occurs at a regularly scheduled visit, that visit may become the End-of-treatment Visit rather than having the patient return for an additional visit.

The electronic case report form (eCRF) should be completed, giving the date and reason for stopping the treatment. If a withdrawal occurs, or if the patient fails to return for visits, the Investigator must determine the primary reason for a patient's premature withdrawal from the trial and record this information on the End-of-treatment eCRF page. The End-of-treatment Visit is not considered the end of the trial.

Next-line therapy may be started after the End-of-treatment Visit at the Investigator's discretion.

If the patient discontinued the randomized IMP treatment due to PD, patients in both treatment groups will have the option to start treatment with CAM2029 20 mg once weekly in an Extension Treatment Period (see further details in Section [000285]).

### 4.1.4 Open-Label Extension Treatment Period (Optional)

After the BIRC confirms PD during treatment with the IMP (CAM2029/comparator) in the Randomized Treatment Period, patients in both treatment groups may be switched to receive treatment with CAM2029 20 mg once weekly in an Extension Treatment Period, if the Investigator considers it beneficial for the patient. The End-of-treatment Visit may be the same visit as the first visit in the Extension Treatment Period. During this period, tumor progression will be evaluated every 12 weeks. Safety will be evaluated continuously and plasma samples for assessment of octreotide concentration will be taken. Patient-reported treatment satisfaction will be assessed using a general questionnaire and health-related quality of life parameters will be assessed by PROs.

Patients may self-administer CAM2029 or have CAM2029 administered by their partner. For patients who received treatment with a comparator product during the Randomized Treatment Period, the feasibility of self- or partner-administration of CAM2029 will be assessed by a trained trial personnel before self- or partner administration of CAM2029 once weekly may be performed. If CAM2029 is not self- or partner-administered, trial personnel will perform the administrations at the clinic. Self- or partner-administration at home is allowed after 3 supervised and successful administrations.

Treatment with CAM2029 will continue until the patients experience any of the following:
Disease progression as confirmed by the BIRC
Unacceptable toxicity that precludes further treatment
Discontinuation of treatment at the discretion of the Investigator or patient
Lost to follow-up
Death

All patients who have PD (radiologically documented according to RECIST 1.1) determined by the local Investigator will require an expedited tumor response review by the BIRC. In cases of discordance (i.e. the BIRC does not confirm the Investigator's assessment of PD), the patient should continue with CAM2029 treatment (if clinically acceptable) until progression has been determined by the BIRC.

After discontinuation of the CAM2029 once-weekly treatment, the patient will be asked to return for an End-of-extension-treatment Visit (see Section [000291]).

### 4.1.5 End-of-Extension-Treatment Visit

Patients who discontinue treatment with CAM2029 once weekly during the Extension Treatment Period should be scheduled for an End-of-extension-treatment Visit within 28 days after the date the CAM2029 treatment is discontinued, at which time all of the assessments listed for the End-of-extension-treatment Visit in Table 6 will be performed. If the decision to discontinue occurs at a regularly scheduled visit, that visit may become the End-of-extension-treatment Visit rather than having the patient return for an additional visit.

The eCRF should be completed, giving the date and reason for stopping the treatment. If a withdrawal occurs, or if the patient fails to return for visits, the Investigator must determine the primary reason for a patient's premature withdrawal from the trial and record this information on the End-of-extension-treatment eCRF page. The End-of-extension-treatment Visit is not considered the end of the trial.

Next-line therapy may be started after the End-of-extension-treatment Visit at the Investigator's discretion.

### 4.1.6 Safety Follow-Up Period

After discontinuation of IMP treatment (in the Randomized Treatment Period or the Extension Treatment Period), patients will be followed for safety for up to 56 days after the last dose of IMP, except if the patient dies, is lost to follow-up, or withdraws consent. At the Safety Follow-up, AEs that were ongoing at the End-of-treatment/End-of-extension-treatment Visit will be followed up and new SAEs assessed as related to the IMP will be recorded.

### 4.1.7 Efficacy Follow-Up Period

Patients who discontinue treatment for reasons other than disease progression, death, lost to follow-up or withdrawal of consent must continue to undergo tumor assessments and assessments of PROs every 12 weeks until one of the following occurs:
Disease progression as confirmed by the BIRC
Withdrawal of consent
Lost to follow-up
Death

At that time, the reason for trial completion should be recorded in the eCRF. If a patient starts a new anti-neoplastic therapy before progression, every attempt should be made to perform tumor evaluation until disease progression. All n-m anti-neoplastic therapies given after the last dose of the IMP must be recorded in the eCRF until disease progression, death, lost to follow-up, or withdrawal of consent occur.

### 4.1.8 Survival Follow-Up

Patients who discontinue IMP treatment and/or the Efficacy Follow-up Period will enter a Survival Follow-up Period during which survival will be collected every 12 weeks or earlier if a survival update is required to meet safety or regulatory needs. During the Survival Follow-up, subsequent anti-neoplastic therapies initiated after IMP discontinuation will be collected along with the start/end date and date of disease progression on subsequent therapies to assess time to progression on next-line therapy (PFS2). The patients will continue to be followed for survival, regardless of treatment discontinuation reason, until death, lost to follow-up, or withdrawal of consent to Survival Follow-up.

Survival information can be obtained via phone, and information will be documented in the source documents and relevant eCRFs.

### 4.2 Rationale of Overall Trial Design, Trial Population, Endpoints and Dose

### 4.2.1 Overall Trial Design and Choice of Comparators

This is a randomized, open-label, parallel-group, active-controlled, multi-center trial evaluating the efficacy of 20 mg CAM2029 administered every 2 weeks versus the Investigator's choice of comparator, octreotide LAR 30 mg or lanreotide ATG 120 mg administered every 4 weeks. The purpose of this trial is to compare the antitumor activity of CAM2029 versus the comparator products (octreotide LAR or lanreotide ATG) in patients with advanced GEP-NET. The comparator products are widely approved in NET for symptom control (flushing and diarrhea) and, in many countries, also for delaying the time to progression.

Patients will be randomized in a 1:1 ratio and the randomization will be stratified based on Ki-67 index of the tumor, site of tumor origin, and choice of comparator product. Stratification is performed to ensure balance in key factors with potential impact on the PFS between treatment groups and to account for a potential difference in anti-tumor efficacy of the comparator products. Because the primary endpoints (PFS) is evaluated by a BIRC and because the syringes and dosing intervals differ between CAM2029 and the comparator products, an open-label design is considered feasible.

An interim analysis for overall survival is planned at the time of the primary PFS analysis. The final analysis of overall survival will be conducted at the latest 2 years after the PFS analysis. Due to long expected overall survival in this population, the final analysis of overall survival will be time-driven, rather than event-driven (31).

When PD has been confirmed for a patient in the Randomized Treatment Period, patients in both treatment groups have the possibility to switch to treatment with CAM2029 once weekly, to investigate potential treatment benefits of more frequent dosing with octreotide. Recent reviews of the literature and results from clinical trials suggest that increasing the dose or the dosing frequency of SSAs may provide additional therapeutic effect in treatment of progressive NET (16, 21, 22, 27, 28). An increase in the dosing frequency of SSAs will also be an alternative to switching to a next-line treatment.

### 4.2.2 Selection of Trial Population

The target population in the current trial comprises adult patients with histologically confirmed, advanced (unresectable and/or metastatic), and well-differentiated NET of presumed GEP origin. SSAs (octreotide and lanreotide) are often used as first-line therapy in patients with advanced GEP-NET. However, there is currently no standard of care for patients with well-differentiated, Grade 3 NET. For patients whose disease expresses somatostatin receptors, use of SSA can be considered. Although patients with well-differentiated Grade 3 NET were not included in the CLARINET trial (which was restricted to patients with GEP-NET with well/moderately differentiated histology and Ki-67 index of less than 10%) or the PROMID trial (which enrolled patients with well-differentiated midgut NET, of whom over 90% had disease with Ki-67 index of ≤2%), SSAs may still have an antiproliferative effect in patients with higher grade disease.

### 4.2.3 Selection of Endpoints

Overall survival is considered the most reliable oncology endpoint but would be challenging to use as the primary endpoint in this patient population, as the overall survival is relatively long. Survival trials in general include long follow-up periods and subsequent cancer therapy that potentially confound the survival analysis, which makes the analyses difficult to perform (32).

Instead, PFS, defined as the time from randomization to the first documented progression or death (whichever occurs first), will be used. PFS is a recommended primary endpoint for NET trials (33) and has been used in many NET trials investigating effects on tumor proliferation (15, 16, 21, 23). This endpoint requires smaller sample sizes and shorter trials as compared to e.g. overall survival. The primary PFS endpoints will be assessed by a BIRC using standard criteria (RECIST 1.1). As a secondary endpoint, PFS assessed by local Investigators will also be evaluated. Other secondary efficacy endpoints in the trial include commonly used endpoints in NET trials, such as overall survival, overall response rate (ORR), disease control rate (DCR), and time to tumor response. In addition, octreotide plasma concentration and the need for symptomatic rescue treatment will be assessed in the trial and the proportion of patients/partners who are judged competent to self- or partner-administer CAM2029 will be evaluated. Health-related quality of life and patient satisfaction with treatment will be assessed using selected PROs. As exploratory endpoints, PFS-ext (defined as the time from date of randomization to documented progression or death from any cause in the Extension Treatment Period, whichever occurs first, and) PFS2 (defined as time from date of randomization to documented progression as per RECIST 1.1 on next-line therapy or death from any cause, whichever occurs first) will be evaluated. In addition, the immunogenicity of CAM2029 will be assessed.

### 4.2.4 Dose Rationale

Non-clinical and clinical data supporting the use of SSAs to control tumor growth have been available for several years. This premise was confirmed by prospective data from 2 randomized controlled trials (14, 15), which showed that 30 mg octreotide and 120 mg lanreotide were effective in improving PFS with doses used for symptom control.

The selection of the CAM2029 dose is based on the results of completed Phase 1 and Phase 2 trials in healthy volunteers and in patients with acromegaly or NET. A dose of 20 mg CAM2029 every 4 weeks has been identified as a suitable starting and maintenance dose for the ongoing Phase 3 trials in patients with acromegaly. However, for patients with NET, a higher octreotide exposure is proposed to reach therapeutic concentrations. In this trial, 20 mg of CAM2029 will be administered every 2 weeks until the patient experiences PD. Thereafter, the patient will have the option to increase the dosing frequency to 20 mg CAM2029 once weekly, if further increased dosing with CAM2029 is considered possibly beneficial for the patient.

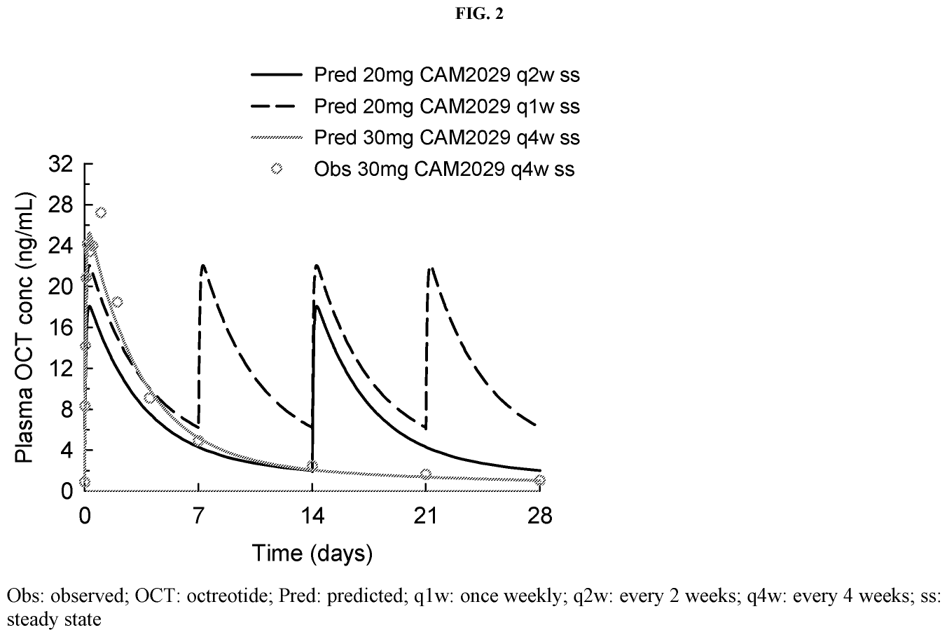

The predicted systemic exposure (maximum plasma concentration at steady state [C_{max,ss}] and area under the plasma concentration-time curve at steady state [AUCₛₛ]) of octreotide for CAM2029 20 mg administered every 2 weeks and CAM2029 20 mg administered once weekly were compared with observed exposure levels in healthy volunteers previously treated with CAM2029 and with obtained exposure levels in the 6-month toxicity study in dogs (study TO-07-278). A population PK model of octreotide for CAM2029 was developed for prediction of octreotide plasma concentrations and systemic exposure after administration of CAM2029. The PK model was developed by nonlinear mixed effects modelling based on data in the clinical trial HS-11-411 (data on file). Predicted octreotide plasma concentrations for the treatment regimens 20 mg CAM2029 every 2 weeks and 20 mg CAM2029 once weekly are shown in FIG. 2 and predicted PK parameters at steady state are presented in Table 3. Observed PK data for 30 mg CAM2029 every 4 weeks (trial HS-11-411) are added in FIG. 2 and Table 3, as a comparison. The predicted C_{max,ss} for 20 mg CAM2029 every 2 weeks (18 ng/mL) and 20 mg CAM2029 once weekly (22 ng/mL) are below the observed C_{max,ss} for 30 mg CAM2029 every 4 weeks (geometric mean 28.5 ng/mL, trial HS-11-411), in which such exposure was well tolerated by the subjects. Additionally, the predicted C_{max,ss} values are below the C_{max,ss} at the no-observed-adverse-effect-level (NOAEL) of 60 mg CAM2029 every 4 weeks in the 6-month study in dogs (mean C_{max,ss} of 101.6 ng/mL; study TO-07-278).

The predicted systemic exposure (AUCₛₛ), described as average plasma concentration during a dosing interval at steady state (C_{av,ss}=AUCₛₛ/dose interval), for 20 mg CAM2029 every 2 weeks (6.4 ng/mL) is somewhat higher than that observed for 30 mg CAM2029 every 4 weeks (geometric mean 5.09 ng/mL, Table 3). However, it is lower than the C_{av,ss} at the NOAEL in the 6-month study in dogs (mean C_{av,ss} equal to 9.6 ng/mL based on AUCₛₛ of 6470 ng*h/mL and a dosing interval of 672 hours at 60 mg CAM2029 every 4 weeks; study TO-07-278) with a safety margin of 1.5. For the 20 mg CAM2029 once weekly treatment, the predicted C_{av,ss} (13 ng/mL, Table 3) is slightly higher than the C_{av,ss} at the NOAEL (9.6 ng/mL).

TABLE 3
Observed or predicted PK parameters of octreotide after
administration of 2 doses of 20 mg CAM2029 every 2 weeks,
4 doses of 20 mg CAM2029 once weekly, and 1 dose of 30
mg CAM2029 (trial HS-11-411) to healthy volunteers

| Treatment | C_{max, ss} | AUCₛₛ | C_{av, ss} |
|---|---|---|---|
| CAM2029 | (ng/mL) | (ng*h/mL) | (ng/mL) |
| 20 mg q2w | 18^{a} | 2150^{a} | 6.4^{a} |
| 20 mg q1w | 22^{a} | 2180^{a} | 13^{a} |
| 30 mg q4w | 28.5^{b} | 3420^{b} | 5.09^{b} |

AUCₛₛ: area under the plasma concentration-time curve at steady state;
C_{av, ss}: average plasma concentration during a dosing interval at steady state;
C_{max, ss}: maximum plasma concentration at steady state;
PK: pharmacokinetic;
q1w: once weekly;
q2w: every 2 weeks;
q4w: every 4 weeks
^{a}Predicted
^{b}Observed data in trial HS-11-411

### 4.3 Trial Duration

Estimated planned first patient first visit: Q2 2021.

Estimated planned last patient first visit: Q4 2022.

### 4.4 End of Trial Definition

Following the primary analysis time point, the trial will remain open. Patients still being followed on the trial will continue as per the schedule of assessments (Table 5 and Table 6).

Patients who are still ongoing in the control treatment group at the time of the primary analysis will have the option to switch to treatment with CAM2029 20 mg every 2 weeks, if the trial meets the primary objective.

The overall survival follow-up will end at the latest 2 years after the primary PFS analysis. At that time, the final analysis of trial data will be conducted. All available data from all patients up to that cut-off date will be analyzed.

The overall end of trial is defined as the last protocol-specified contact with the last patient ongoing in the trial.

At the end of the trial, efforts will be made to continue provision of CAM2029 outside this trial through an alternative setting to patients who, in the Investigator's opinion, are still deriving clinical benefit.

### 5 Selection of Trial Population

### 5.1 Planned Number of Patients

Approximately 300 patients will be randomized, with 150 patients in the CAM2029 treatment group and 150 patients in the comparator treatment group.

### 5.2 Inclusion Criteria for the Trial

Patients meeting each of the following criteria at screening (unless otherwise specified) will be eligible to participate in the clinical trial:
1. Voluntary and valid written informed consent to participate in the trial obtained before any other trial-related procedures are performed
2. Male or female patient ≥18 years old
3. Histologically confirmed, advanced (unresectable and/or metastatic), and well-differentiated NET of GEP or presumed GEP origin
4. At least 1 measurable lesion according to RECIST 1.1 determined by multiphasic CT or MRI (performed within 28 days before randomization)
5. Expression of somatostatin receptors on lesions documented by CT/MRI scans, assessed by somatostatin-receptor imaging modalities within 3 months before randomization
6. Results from FDG-PET CT for patients with well-differentiated Grade 3 NET (if performed) must show that FDG avid areas of disease also are avid on somatostatin-receptor imaging
7. Eastern Cooperative Oncology Group (ECOG) performance status of 0 to 2
8. Having the following laboratory values:
   a. Absolute neutrophil count ≥1.5×10⁹/L
   b. Platelets ≥75×10⁹/L
   c. Hemoglobin ≥9 g/dL
   d. Total bilirubin ≤1.5×upper limit of normal (ULN)*
   e. Aspartate aminotransferase (AST)≤3.0×ULN (if hepatic metastases: ≤5.0×ULN)
   f. Alanine aminotransferase (ALT)≤3.0×ULN (if hepatic metastases: ≤5.0×ULN)
   g. Creatinine clearance ≥40 mL/min defined by the Cockcroft-Gault equation
   *Patients with previous diagnosis of Gilbert's syndrome may be included if the disease is not accompanied by other hepatobiliary disorders and if the total bilirubin is <3 mg/dL (≤51.3 gmol/L) and the direct bilirubin is ≤ULN
9. Female patients of childbearing potential must be willing to use an acceptable method of contraception from screening to the Safety Follow-up Visit (see Section [000470])

### 5.3 Exclusion Criteria for the Trial

Patients meeting any of the following criteria will not be eligible to participate in the clinical trial:
1. Poorly differentiated NEC, adenocarcinoid, goblet cell carcinoid, large cell NEC, small cell carcinoma, or mixed tumor
2. Previously diagnosed with multiple endocrine neoplasia Type 1
3. Tumor with primary origin outside the GEP tract
4. Documented evidence of disease progression while on treatment (including SSAs) for locally advanced unresectable or metastatic disease
5. Known central nervous system metastases
6. Consecutive treatment with long-acting SSAs for more than 6 months before randomization
7. Carcinoid symptoms that are refractory to treatment (according to the Investigator's judgement) with conventional doses of octreotide LAR or lanreotide ATG and/or to treatment with daily doses of ≤600 μg of octreotide IR
8. Previous treatment with more than 1 cycle (where 1 cycle means ≤28 days on treatment) of targeted therapies such as mTOR inhibitors (e.g. sirolimus, temsirolimus, or everolimus) or vascular endothelial growth factor inhibitors (e.g. sunitinib, lenvatinib, or cabozantinib), or more than 1 cycle of chemotherapy or interferon for GEP-NET
9. Treatment of GEP-NET with trans-arterial chemoembolization or trans-arterial embolization within 12 months before screening
10. Previously received radioligand therapy (PRRT) at any time
11. History of another primary malignancy, except the following:
   a. Stable and well-differentiated microcarcinoma of the thyroid
   b. Non-melanoma skin cancer or carcinoma in situ of the cervix, uterus or breast, from which the patient has been disease-free for ≥3 years
   c. Fully resected, non-metastatic melanoma
   d. A primary malignancy that has been completely resected and is in complete remission for ≥5 years
12. Major surgery/surgical therapy for any cause within 1 month before screening, surgical therapy of loco-regional metastases within 3 months before screening, or minor surgery within 14 days before screening. Patient must have recovered from the treatment and be in good clinical condition
13. Hepatic/pancreatic-related exclusion criteria:
   a. Active hepatitis. Patients with no significant viral load, no acute signs of inflammation, and no clinical necessity for therapy are allowed, at the Investigator's discretion
   b. Known gallbladder or bile duct disease, or acute or chronic pancreatitis
   c. Symptomatic cholelithiasis
   d. Clinically active or chronic liver disease, including liver cirrhosis of Child-Pugh class B or C
14. Patients with poorly controlled diabetes, as evidenced by hemoglobin A1c (HbA1c)>8.0%
15. Cardiac history or current diagnosis of cardiac disease indicating significant risk of safety for patients participating in the trial, such as uncontrolled or significant cardiac disease, including any of the following:
   a. History of myocardial infarction, unstable angina pectoris, or coronary artery bypass graft within 6 months before screening
   b. Uncontrolled congestive heart failure
16. Clinically significant cardiac arrhythmias (e.g. ventricular tachycardia), complete left bundle branch block, or high-grade atrioventricular block (e.g. bifascicular block, Mobitz type II, and third-degree atrioventricular block)
17. Long QT syndrome, family history of idiopathic sudden death or congenital long QT syndrome, or any of the following:
   a. Risk factors for Torsades de Pointes, including uncorrected hypokalemia or hypomagnesemia, history of cardiac failure, or history of clinically significant/symptomatic bradycardia
   b. Treatment with concomitant medication(s) with a “known risk of Torsades de Pointes” per www.crediblemeds.org that cannot be discontinued or replaced with safe alternative medication at least 7 days or 5 half-lives (whichever is longer) before start of IMP treatment
   c. Patients with a baseline QTc interval corrected by Fridericia's formula (QTcF)>450 msec for males and >470 msec for females at screening
18. Presence of active or suspected acute or chronic uncontrolled infection, including active human immunodeficiency virus (HIV) infection, or with a history of compromised immune system
19. Pregnant, lactating, or planning to become pregnant during the trial
20. Any known allergy, hypersensitivity, or intolerance to octreotide, lanreotide, or any related drug, or history of any drug hypersensitivity or intolerance that, in the Investigator's opinion, would compromise the safety of the patient
21. Clinically significant laboratory abnormalities at screening, which, in the Investigator's opinion, may prevent the patient from safely participating in the trial
22. Any other contraindicated serious medical condition that, in the Investigator's opinion, may prevent the patient from safely participating in the trial
23. Any other current or prior medical condition that may interfere with the conduct of the trial or the evaluation of its results in the opinion of the Investigator or the Sponsor's Medical Monitor
24. Unwilling or unable to comply with the requirements of the protocol or in a situation or condition that, in the Investigator's opinion, may interfere with participation in the trial
25. Participation in any other clinical trial to test an investigational drug or device within the 3 months before screening
26. On the staff, affiliated with, or a family member of the personnel directly involved with this trial

### 5.4 Inclusion Criteria for the Open-Label Extension Treatment Period

Patients need to fulfil the following criteria to proceed to treatment with CAM2029 20 mg once weekly in the Extension Treatment Period:
1. Disease progression confirmed by the BIRC
2. Voluntary and valid written informed consent to participate in the Extension Treatment Period obtained before any procedures are performed
3. At least 6 months of treatment with IMP (CAM2029/comparator) in the Randomized Treatment Period before documented disease progression
4. Female patients of childbearing potential must be willing to use an acceptable method of contraception until the Safety Follow-up Visit (see Section [000470])

### 5.5 Exclusion Criteria for Open-Label Extension Treatment Period

Patients meeting any of the following criteria may not participate in the Extension Treatment Period:
1. Unresolved, drug-related SAE that, in the Investigator's opinion, contraindicates treatment with CAM2029
2. Clinically significant symptoms, medical conditions, rapid clinical deterioration, or other circumstances that, in the Investigator's opinion, would preclude compliance with the protocol, adequate cooperation in the trial, or may prevent the patient from safely participating in the trial
3. Pregnancy

### 5.6 Lifestyle Considerations

No dietary or physical restrictions are required during the trial.

### 5.7 Screen Failures and Re-Screening

Screen failures are defined as patients who consent to participate in the trial but are not subsequently randomized in the trial. A minimal set of screen failure information is required to ensure transparent reporting of screen failure participants to meet the publishing requirements of the Consolidated Standards of Reporting Trials and to respond to queries from regulatory authorities. Minimal information includes demography, eligibility criteria, possible other screen failure information, and any SAE.

Patients who do not meet the eligibility criteria may be screened one more time and should receive a new screening number. If the patient is re-screened, the informed consent form (ICF) must be re-signed before re-screening procedures are performed. The eCRF should clearly indicate that the patient has been re-screened and the original screening number. However, if e.g. abnormal laboratory findings are considered by the Investigator to be temporary and not reflective of the patient's usual state, the patient may be re-tested once within the screening period (keeping the original screening number). The decision to re-screen or re-test will be made on a case by case basis by the Investigator in consultation with the Medical Monitor. Applicable screening information and results obtained during the first screening may be used for eligibility assessments during re-screening.

### 6 Treatments

### 6.1 Treatments Administered

The treatments to be administered in in the Randomized Treatment Period of this trial are described in Table 4.

TABLE 4
Trial treatments in the Open-label Randomized Treatment Period

| Treatment | CAM2029 treatment | | | Rescue medication |
|---|---|---|---|---|
| group name | group | Comparator treatment group | | (both groups) |
| Treatment name | CAM2029 (octreotide | Octreotide LAR | Lanreotide ATG | Octreotide IR |
| | subcutaneous depot) | | | |
| IMP or NIMP | IMP | IMP | IMP | NIMP |
| Dosage | Solution for injection | Powder and solvent | Solution for | Solution for |
| formulation | in a ready-to-use pre- | for suspension for | injection in a ready- | injection/infusion |
| | filled syringe | injection | to-use pre-filled | |
| | | | syringe | |
| Dosage levels | 20 mg every 2 weeks | 30 mg every | 120 mg every | 50, 100 or 500 μg up |
| | | 4 weeks | 4 weeks | to 600 μg per day |
| Route of | SC injection | IM injection | Deep SC injection | SC injection |
| administration | | | | |

ATG: autogel;
IM: intramuscular;
IMP: investigational medicinal product;
IR: immediate-release;
LAR: long-acting release;
NIMP: non-investigational medicinal product;
SC: subcutaneous

In the Extension Treatment Period, CAM2029 20 mg will be administered once weekly as an SC injection. Rescue medication may also be used in the same way as in the Randomized Treatment Period.

### 6.2 IMP Administration

### 6.2.1 CAM2029

### Randomized Treatment Period

Patients who are randomized to the CAM2029 treatment group in the Randomized Treatment Period will receive 20 mg CAM2029 as an SC injection every 2 weeks in the abdomen or thigh regardless of who is giving the injection (trial personnel, partner, or patient).

If the date of a visit does not conform to the schedule, subsequent visits should be planned to maintain the visit schedule relative to the Day 1 visit. The injections should not be administered in a recently used injection site. The site of injection will be recorded. An injection site pictorial and specific instructions for use are included in the Manual of Procedures. The dose, date and exact time of dosing must be recorded in the eCRF.

### Self- or Partner-Administration of CAM2029

CAM2029 may be self-administered by the patient or administered by the patient's partner. A “partner” may be the patient's spouse, parent, child, or sibling etc. or any other person that the patient trusts to administer the injection. Self- or partner-administration of CAM2029 in the abdomen or thigh is allowed after appropriate training under the supervision of adequately trained trial personnel. The first self- or partner-administration should preferably be performed on Day 1. The trial personnel will document that the patient or partner understands the administration instructions, performs the injection correctly, and administers a full dose (see the Manual of Procedures for a checklist of the feasibility of self- or partner-administration). If the patients or their partners are considered competent to administer CAM2029, they may continue with the self- or partner-administration. If CAM2029 is not self- or partner-administered, trial personnel will administer CAM2029. If the partner who is administering CAM2029 is replaced, the new partner must undergo appropriate training, as described above.

### Home-Administration of CAM2029

The first 3 doses will be self- or partner-administered at the clinic. Starting from the second dose in Month 2, CAM2029 may be self- or partner-administered at home on Day 15 of each month. The Investigator will dispense an appropriate number of investigational treatment packages for home administrations. The patients will use a patient diary to record the dose(s), date(s), and exact time(s) of administration, the injection site, and who performed the injection. At the next visit, the patients will return the used syringes and packaging to the trial personnel. The trial personnel will document the administration dates and confirm whether the full dose was administered. Detailed instructions will be provided separately. Patients will be asked to return any unused medication and packaging on a regular basis or at the End-of-treatment Visit at the latest to ensure proper drug accountability.

It is important that patients contact the Investigator/trial personnel if they experience any AE/SAE or have any concerns, or if there are any problems with the medication (e.g. if there is something wrong with the syringe), during the period of CAM2029 administration at home. If the patients (or their partners) are not able, confident, or willing to administer the treatment at home, CAM2029 should be administered by the trial personnel.

Patients should be instructed not to make up missed doses. A missed dose during treatment every 2 weeks is defined as a case when the full dose is not taken ±3 days from the scheduled day of the dosing. If a dose is missed, the patient should continue treatment with the next scheduled dose as planned.

### Extension Treatment Period

Patients who self- or partner-administered CAM2029 20 mg every 2 weeks in the Randomized Treatment Period may continue to self- or partner-administer CAM2029 20 mg once weekly during the Extension Treatment Period. CAM2029 will be injected in the abdomen or in the thigh. Self- or partner-administration at home may be performed from the second dose in Month 1.

For patients who received a comparator product in the Randomized Treatment Period, the first self- or partner-administration of CAM2029 in the Extension Treatment Period should preferably be performed on Day 1 of the Extension Treatment Period. Trained trial personnel will document that the patient or partner understands the administration process, performs the injection correctly and administers a full dose (see detailed instructions above). The first 3 doses will be self- or partner-administered at the clinic. Starting from the fourth dose in Month 1, the patients may self-administer CAM2029 or receive the administration of CAM2029 from their partner at home.

If CAM2029 is administered at home during the Extension Treatment Period, the Investigator will dispense an appropriate number of investigational treatment packages for home administrations. The patients will record the dose(s), date(s) and exact time(s) of administration, the injection site and who performed the injection in a patient diary. At the next visit, the patients will return the used syringes and packaging to the trial personnel. The trial personnel will document that the patient or partner understands the administration instructions, performs the injection correctly, and administers a full dose. Detailed instructions will be provided separately. Patients will be asked to return any unused medication and packaging on a regular basis or at the End-of-extension-treatment Visit at the latest to ensure proper drug accountability.

It is important that patients contact the Investigator/trial personnel if they experience any AE/SAE or have any concerns, or if there are any problems with the medication (e.g. if there is something wrong with the syringe), during the period of CAM2029 administration at home. If the patients (or their partners) are not able, confident, or willing to administer the treatment at home, CAM2029 should be administered by trial personnel.

Patients should be instructed not to make up missed doses. A missed dose during the once-weekly treatment is defined as a case when the full dose is not taken ±2 days from the scheduled day of the dosing. If a dose is missed, the patient should continue treatment with the next scheduled dose as planned.

### 6.2.2 Comparator Products

### Octreotide LAR

Octreotide LAR 30 mg will be administered every 4 weeks in the Randomized Treatment Period by a trained HCP. Octreotide LAR will be administered as an IM injection in the gluteus as per local practice and regulation. Alternating injections sites (right and left gluteus) should be used for the injections.

### Lanreotide ATG

Lanreotide ATG 120 mg will be administered every 4 weeks in the Randomized Treatment Period by a trained HCP. Lanreotide ATG will be administered as a deep SC injection in the gluteus or thigh as per local practice and regulation. Alternating injections sites (right and left gluteus or thigh) should be used for the injections.

### 6.2.3 Dose Delay of IMP

Recommendations for dose delay/interruption of IMP (CAM2029 or comparator product) in case of ADRs or QTcF prolongation are included in Section 0, Appendix 1. All dose delays should preferably be discussed with the Medical Monitor before implementation.

### 6.3 NIMP Administration

Octreotide IR as rescue medication (non-investigational medicinal product [NIMP]) may be self-injected for symptom rescue as per local practice during the trial and at the Investigator's discretion, up to a maximum of 600 μg per day. Patients who are unable to achieve symptom control at the maximum allowed dose are advised to contact their Investigator for guidance. Administration of octreotide IR must be avoided within 24 hours before each clinic visit.

The patients will record the dose(s), date(s), and exact time(s) of administration of octreotide IR in a patient diary.

### 6.4 Characteristics and Source of Supply, Packaging and Labelling

The Sponsor will provide CAM2029 is and it will be handled according to the principles of Good Manufacturing Practice. The labelling will comply with applicable regulatory requirements.

The Sponsor will provide octreotide LAR and lanreotide ATG through a central vendor, who will obtain the marketed drug and label it in accordance with applicable regulatory requirements. The vendor will be responsible for distribution of octreotide LAR and lanreotide ATG to the trial sites.

Octreotide IR as rescue medication (NIMP) will be prescribed by the Investigator as per local practice.

### 6.5 Conditions for Storage

The IMP (CAM2029 and comparator products) must be received by designated personnel at the trial site, handled and stored safely and properly, and kept in a secured location to which only the Investigator and designated trial personnel have access. The IMP should be stored in accordance with the storage conditions specified on the labels.

Octreotide IR should be handled and stored according to local product labels.

### 6.6 Method of Assigning Patients to Treatment Groups

### 6.6.1 Recruitment

Patients will be recruited by methods chosen at the discretion of the sites. Each site will maintain a screening log of all screened patients.

### 6.6.2 Randomization and Blinding

This is an open-label trial. To minimize bias, patients fulfilling the eligibility criteria will be randomized in a 1:1 ratio to 1 of the treatment groups (CAM2029 or comparator), using an interactive randomization system (interactive web or voice response system). Before the trial is initiated, the log-in information and directions for the interactive randomization system will be provided to each trial site. The Investigator will prospectively choose either octreotide LAR or lanreotide ATG as the potential comparator drug before the patient is randomized into the trial.

Randomization will be stratified by the following:
Histological grade Ki-67<10% versus Ki-67≥10%
Tumor origin (pancreas versus other GI origin)
Intended choice of comparator (octreotide LAR or lanreotide ATG)

### 6.7 Treatment Compliance

### 6.7.1 Dispensing and Accountability

Only eligible patients participating in the trial will receive IMP. Only authorized trial personnel may dispense the IMP to the patients. Once dispensed, the IMP must not be relabeled or reassigned for use by other patients.

The Investigator (or his/her designated personnel) will maintain an Injection Log detailing the dates and quantities of IMP administered to each patient, as well as an Accountability Log detailing dates and quantities of CAM2029 dispensed to and returned by each patient in case of administration at home. The monitor will verify drug accountability during the trial.

### 6.7.2 Assessment of Compliance

CAM2029 will be self- or partner-administered or administered by designated trial personnel during the trial. Patients will be allowed to self-administer or to have CAM2029 administered by his/her partner from the first injection. Starting with the second injection in Month 2 in the Randomized Treatment Period or the fourth injection in Month 1 in the Extension Treatment Period (for patients who received comparator products in the Randomized Treatment Period), patients may self-administer or have CAM2029 administered by his/her partner at home. The patients will be given a diary for recording of the injections at home.

All administrations of comparator products will be performed by an HCP.

Dosing compliance will be recorded by the Investigator or designee. Treatment dates, including dates for treatment delays, will also be recorded in the eCRF.

On PK sampling days, compliance will be assured by administration of the IMP at the clinic and compliance will also be verified by determination of octreotide in plasma in patients receiving CAM2029 or octreotide LAR.

Patients will receive diaries for recording of self-administration of octreotide IR.

### 6.8 Return and Destruction

If the patient is self- or partner-administering CAM2029 at home, they will be instructed to return used and unused CAM2029 to the trial personnel on a regular basis and at the latest at the End-of-treatment/End-of-extension-treatment Visit. The trial personnel will ensure that the appropriate dose of CAM2029 is administered and that drug accountability is performed.

All used IMP can be destroyed at the trial site (in accordance with local requirements) after the drug accountability has been finalized and signed off by the Investigator.

All unused IMP will be accounted for and must be destroyed in a certified way after approval by the Sponsor (either at the trial site or any other certified site, such as depot).

### 6.9 Product Quality Complaints and Device Malfunctions

A Product Quality Complaint (PQC) is any written, electronic, or oral communication that alleges deficiencies related to device malfunctions or the identity, quality, stability, reliability, or safety of a product, including its labeling, delivery system, or packaging integrity.

Any PQC discovered during the initial inventory of the IMP should follow the instructions provided on the receipt letter; no PQC should be filed for issues identified when opening or unpacking a shipment. Any PQC discovered after allocation of an IMP to a patient should be reported as a PQC. Subsequently, any observation of a PQC requires immediate notification within 24 hours after being made aware of the PQC, to the Sponsor via a completed and signed PQC form.

Any IMP associated with a PQC should be quarantined and withheld until further direction is received from the Sponsor. Photographs or physical samples may be requested to support an investigation. The IMP must not be disposed.

In addition, PQC information must be included on the Accountability Log or equivalent in the comments field. The monitor can assist in the event of questions relating to this process.

When enrolling patients into this trial it is the responsibility of the trial site to instruct patients not to use the IMP if they have a concern related to the IMP such as an issue with the labeling, IMP, or package integrity and to immediately report it using contact information provided on the patient ID card or on the ICF.

If the PQC is associated with an AE or an SAE, this should be indicated in the PQC form and the event recorded on the AE page of the eCRF. In case of an SAE, the event must be reported as described in Section [000551].

The Sponsor will assess the PQC and use the information herein to monitor the patient's safety and to improve the safety and performance of the product. The Sponsor is also responsible for notifying the relevant regulatory authorities and Market Authorization Holder of any PQC according to applicable legislation.

### 6.10 Concomitant Medication/Therapies

The patient must notify the trial personnel about any new medications he/she takes after the start of the trial. All medications (other than the IMP and rescue medication) and significant non-drug therapies (including physical therapy, herbal/natural medications, and blood transfusions) administered during the trial must be listed on the concomitant medication page in the eCRF. The Investigator will determine if the concomitant medication(s) affects the patient's eligibility to continue to participate in the trial.

For patients who are switched to a next-line therapy, the therapy must be recorded in the eCRF.

### 6.10.1 Permitted Concomitant Therapies

Supportive care agents and medications required to treat AEs or to manage cancer symptoms or concurrent diseases, e.g. pain medications, pancreatic enzyme replacement, anti-emetics, and anti-diarrheals are allowed.

Octreotide IR may be used for symptom rescue at the Investigator's discretion, see further information in Section [000372]. Administration of octreotide IR must be avoided within 24 hours before each clinic visit.

6.10.2 Permitted Concomitant Therapy Requiring Caution and/or Action

Dose adjustment of medicinal products such as beta blockers, calcium channel blockers, insulin and antidiabetic medicinal products, or agents to control fluid and electrolyte balance may be necessary during SSA administration. In addition, octreotide has been found to reduce the intestinal absorption of cyclosporin and to delay that of cimetidine (30). SSAs also increase the availability of bromocriptine (30, 34).

Limited published data indicate that SSAs might decrease the metabolic clearance of compounds known to be metabolized by cytochrome P450 (CYP) enzymes, which may be due to the suppression of growth hormone. Since it cannot be excluded that octreotide may have this effect, other drugs mainly metabolized by CYP3A4 and which have a low therapeutic index (e.g. quinidine and terfenadine) should therefore be used with caution (30, 35).

Medications with a possible risk of causing Torsades de Pointes should be used with caution by patients until the Safety Follow-up Visit (see further information, including a list of drugs with a possible risk of Torsades de Pointes, on the Advisory Board of the Arizona CERT website: www.crediblemeds.org). Interruption of IMP treatment may be considered if the concomitant medication is only needed for a short time.

### Palliative Radiotherapy

Palliative radiation is permitted if performed solely for bone pain relief but should not be delivered to a target lesion. If palliative radiotherapy is initiated after the start of IMP treatment, the reason for its use must be clearly documented and progression as per RECIST 1.1 must be ruled out.

### Use of Bisphosphonates

Bisphosphonates are generally allowed with the following caveats:
Chronic concomitant bisphosphonate therapy for the prevention of bone metastases is not permitted
Bisphosphonate therapy for the treatment of osteoporosis is permitted
Bisphosphonate therapy for the prevention of skeletal-related events in patients with bone metastases is permitted
If bisphosphonate therapy is started after the first dose of IMP, disease progression should be formally ruled out by appropriate imaging prior to the initiation of bisphosphonates

### 6.10.3 Prohibited Concomitant Therapy

The following treatments are NOT allowed after the start of the trial:
Anti-neoplastic therapy such as chemotherapy, targeted therapies (e.g. everolimus, sunitinib, and bevacizumab), PRRT, and interferon are not permitted until the End-of-treatment/End-of-extension-treatment Visit
Other investigational drugs or therapies until the Survival Follow-up
Treatment with drugs with a known risk of Torsades de Pointes is prohibited from 7 days before screening to the Safety Follow-up Visit (see further information, including a list of drugs with a known risk of Torsades de Pointes, on the Advisory Board of the Arizona CERT website: www.crediblemeds.org). If a patient needs to take any of these QT-prolonging drugs, prior IMP discontinuation will be required first. Therefore, other appropriate treatment options should be considered to avoid patient discontinuations as much as possible

### 6.11 Withdrawal Criteria

6.11.1 Withdrawal from Treatment

Patients may voluntarily discontinue from treatment for any reason at any time. If a patient decides to discontinue from treatment, the Investigator must make every effort to determine the primary reason for this decision. The reason for discontinuation should be recorded in the patient's chart and on the appropriate eCRF pages.

Patients who discontinue IMP treatment should NOT be considered withdrawn from the trial. Every effort will be made to keep patients who discontinue IMP treatment in the trial. They should return for the assessments indicated in Table 5 and Table 6. If they fail to return for these assessments for unknown reasons, every effort (e.g. telephone, email, letter) should be made to contact them.

The Investigator should discontinue a patient's IMP treatment if he/she believes that continuation would be detrimental to the patient's well-being or if the patient experiences an intercurrent illness that compromises the patient's ability of to fulfil protocol requirements. The Investigator should contact the Medical Monitor before a patient is withdrawn from treatment.

IMP treatment must be discontinued under the following circumstances:
Emergence of ADRs as per the guidance in Section [000756], Appendix 1
Pregnancy (see Section [000537])
Any protocol deviation (including prohibited concomitant therapy) that results in a significant risk to the patient's safety or that will interfere with the assessment of the efficacy endpoints of this trial, including significant non-compliance with the trial protocol and procedures

In addition to the general withdrawal criteria, the toxicity-related criteria listed below will also require IMP treatment discontinuation. The final decision to discontinue IMP treatment is up to the judgment of the Investigator.

Hepatic-related Discontinuation Criteria
ALT or AST levels >8×ULN
ALT or AST levels >5×ULN for more than 2 weeks (except if the patient has hepatic metastases)
Clinically significant and prolonged symptoms of reduced hepatic function, e.g. appearance of chronic fatigue, nausea, vomiting, right upper quadrant pain or tenderness, itchy skin, or swelling of legs and ankles
The patient is diagnosed with liver cirrhosis of Child-Pugh class C

Re-challenge is not recommended when IMP treatment is discontinued due to the above criteria.

For details on follow-up of potential drug-induced liver injury cases, refer to Section [000758], Appendix 1.

### Cardiac-Related Discontinuation Criteria

QT/QTcF≥501 or >60 msec increase from baseline (average of triplicate ECG) after repeated measurement and as confirmed by central ECG reader
Torsades de Pointes
Polymorphic ventricular tachycardia
Signs/symptoms of serious arrhythmia
Use of QT-prolonging medication with a known risk of Torsade de Pointes
Hypokalemia (<3.5 mmol/L) or hypomagnesemia (<0.7 mmol/L), or clinically significant hypocalcemia confirmed by repeated testing that is either a new finding or accompanied by vomiting or diarrhea and not corrected by treatment

For details on QT prolongation management, refer to Section [000760], Appendix 1.

### Hyperglycemia-related Discontinuation Criteria

Patients with HbA1c≥10% at 3 consecutive visits (including unscheduled visits) despite prior appropriate management should be discontinued from IMP treatment
   6.11.2 Withdrawal from Trial

A patient is free to withdraw his/her consent and discontinue participation in the trial at any time and for any reason. Patients who withdraw consent during a scheduled visit will be asked to complete that visit as the End-of-treatment/End-of-extension-treatment Visit. If they withdraw outside of a scheduled visit, they may be asked if they are willing to attend an End-of-treatment/End-of-extension-treatment Visit. The reason(s) for discontinuation will be appropriately documented.

All efforts will be made to ensure that all patients are informed of the importance of the clinical trial and collection of data they provide. The Sponsor will continue to retain and use all research results already collected for the trial evaluation. Biological samples that have already been collected may be retained until the trial is completed and reported (or as required by local regulations).

The Investigator will be trained about the importance of patient retention and steps to prevent missing data. The Investigator must maintain a record of all patients who discontinue from the trial before completion; the reason(s) for trial discontinuation will be documented. If a patient chooses to withdraw from the trial, the Investigator should make every effort to obtain and record the reason(s) for withdrawal, if possible, although the patient is not obligated to provide such a reason.

Withdrawn patients will not be replaced.

### 6.12 Lost to Follow-up

For patients whose status is unclear because they fail to appear for trial visits without stating an intention to withdraw consent, the Investigator should contact the patient and document the steps taken to do so in the source documents, e.g. dates of telephone calls, registered letters, etc. A patient should not be considered lost to follow-up until due diligence has been completed. Patients who are lost to follow-up should be recorded as such in the eCRF. The time point for the last assessment of survival status for the patient should also be recorded in the eCRF.

### 7. Trial Assessments and Procedures

### 7.1 Trial Procedures and Flow Charts

Trial procedures for patients who participate in the Randomized Treatment Period of the trial and then proceed to the post-treatment follow-up periods are summarized in Table 5. Trial procedures for patients continuing from the Randomized Treatment Period to the Extension Treatment Period and then to the post-treatment follow-up periods are summarized in Table 6 Immediate safety concerns should be discussed with the Medical Monitor immediately upon occurrence or awareness to determine if the patient should continue or discontinue trial intervention.

If applicable and if agreed with the Sponsor, visits or specific assessments may be conducted at a patient's home or at a suitable alternative location by mobile qualified and delegated trial personnel. In such cases, the Investigator remains responsible for oversight and protocol procedure adherence. For this purpose, the terms “clinic visit” and “visit to the clinic” in this protocol may also refer to a remote visit.

TABLE 5
Schedule of trial procedures and assessments for the Open-label Randomized
Treatment Period and the post-treatment and survival follow-up periods

| | Trial Period | | | | | | | |
|---|---|---|---|---|---|---|---|---|
| | | Open-label Randomized Treatment Period | | | | | | |
| | Screen. | Month 1 | | Month 2 | Month 3 | | | Month 4-n |
| | Visit number | | | | | | | |
| | | 11 | 12 | 21 | 31 | 32^{a} | 33^{a,d} | 41-n1 |
| | Day^{e} | | | | | | | |
| | −28 | | | | | | | |
| | to −1 | | | | | | | |
| | or −7 | | | | | | | |
| | to −1^{f} | 1 | 15 | 1 | 1 | 8 | 15 | 1 |
| Screening procedures | | | | | | | | |
| Informed consent | X | | | | | | | |
| Inclusion/exclusion criteria | X | X | | | | | | |
| Demographic data and | | | | | | | | |
| baseline characteristics | | | | | | | | |
| Demographic data | X | | | | | | | |
| GEP-NET medical and | X | | | | | | | |
| treatment history | | | | | | | | |
| (including mitotic rate | | | | | | | | |
| and Ki-67 index) | | | | | | | | |
| Medical history | X | | | | | | | |
| Prior medications | X | | | | | | | |
| ECOG performance status | X^{f} | X | | X | X | | | X |
| Height, BMI | X | | | | | | | |
| Weight | X | X | | X | X | | | X |
| Randomization procedures | | | | | | | | |
| Randomization | | X | | | | | | |
| IMP administration | | | | | | | | |
| CAM2029 administration^{g} | | X | X | X | X | | X | X |
| Comparator administration | | X | | X | X | | | X |
| NIMP administration | | | | | | | | |
| Octreotide IR (rescue | X | X | X | X | X | X | X | X |
| medication)^{h} | | | | | | | | |
| Imaging | | | | | | | | |
| Somatostatin-receptor | X | | | | | | | |
| imaging | | | | | | | | |
| FDG-PETⁱ | X | | | | | | | |
| Tumor evaluation | X^{j} | Every 12 weeks from randomization until disease progression is confirmed by the BIRC | | | | | | |
| Gallbladder ultrasound^{k} | X | | | | | | | M7 and every 6 |
| | | | | | | | | months thereafter |
| Pharmacokinetics | | | | | | | | |
| Plasma samples for octreotide | | X | | X | X | | X | M4, M5, M6, M7, |
| measurement (pre-dose)^{l} | | | | | | | | M9 M10 to Mn: if |
| | | | | | | | | ECG is assessed |
| Plasma samples for octreotide | | X | | | M3D1: | M3D8168 | | M6 and M9: 24 h |
| measurement (post-dose)^{l} | | (24 h) | | | 0.5 h, | h^{m} | | M12 to Mn: 24 h if |
| | | | | | 2 h, | | | ECG is assessed |
| | | | | | 5 h, | | | |
| | | | | | 24 h^{m} | | | |
| Cardiac assessments | | | | | | | | |
| ECG (pre-dose) | X^{f} | X | | X | X | | | M6 and M9; and |
| | | | | | | | | thereafter every |
| | | | | | | | | 3 months only if mean |
| | | | | | | | | QTcF ≥481 msec at any |
| | | | | | | | | time before M10 |
| ECG (24 hours after injection) | | X | | | X | | | M6 and 9; and |
| | | | | | | | | thereafter every |
| | | | | | | | | 3 months only if mean |
| | | | | | | | | QTcF ≥481 msec at any |
| | | | | | | | | time before M10 |
| Laboratory assessments | | | | | | | | |
| Hematology, biochemistry, | X^{f} | X | | X | X | | | X |
| thyroid testing, HbA1c | | | | | | | | |
| Coagulation | X^{f} | | | | | | | Every 3 months |
| Hepatitis (B, C) and HIV | X | | | | | | | |
| Pregnancy serum test | X^{f} | | | | | | | |
| Pregnancy urine test | X | If clinically indicated | | | | | | |
| Immunogenicityⁿ | X | X | | X | | | | M4, M6, M9, M12 and |
| | | (pre- | | (pre- | | | | then every 6 months |
| | | dose) | | dose) | | | | (pre-dose) |
| Other safety assessments | | | | | | | | |
| Physical examination | X^{f} | X | | X | X | | | X |
| Vital signs | X^{f} | X | | X | X | | | X |
| AEs | X | X | X | X | X | X | X | X |
| Concomitant medications and | | X | X | X | X | X | X | X |
| procedures | | | | | | | | |
| PROs | | | | | | | | |
| EORTC QLQ-C30 | | Day 1, every 12 weeks and at End of Treatment, until start of a next-line therapy (if applicable) | | | | | | |
| QLQ-GINET21 | | Day 1, every 12 weeks and at End of Treatment, until start of a next-line therapy (if applicable) | | | | | | |
| SF-36 | | Day 1, every 12 weeks and at End of Treatment, until start of a next-line therapy (if applicable) | | | | | | |
| PGI-S | | Day 1, every 12 weeks and at End of Treatment, until start of a next-line therapy (if applicable) | | | | | | |
| TSQM | | | | | | | | Every 12 weeks and at End of Treatment, until |
| | | | | | | | | start of a next-line therapy (if applicable) |
| Patient exit interview | | | | | | | | |
| Other assessments | | | | | | | | |
| Recording of hospitalizations | | X | X | X | X | | | X |
| Feasibility of self- or partner- | | X | X | At first 3 times of self/partner-administration, | | | | |
| administration of CAM2029^{r} | | | | then collected only if unsuccessful | | | | |
| Recording of reasons for | | X | X | X | X | X | X | X |
| withdrawal of self- or partner- | | | | | | | | |
| administration (as applicable)^{r} | | | | | | | | |
| Discontinuation | | | | | | | | |
| Recording of next-line therapy | | | | | | | | |
| since discontinuation of IMP | | | | | | | | |
| Survival Follow-up | | | | | | | | |

| | Trial Period | | | | |
|---|---|---|---|---|---|
| | Open-label Randomized | | | | |
| | Treatment Period | | Post-treatment | | |
| | Month 2 | End-of- | FU | | |
| | and | treatment | Safety | Efficacy | Survival |
| | Month 4-n^{a} | Visit | FU^{b} | FU^{c} | FU |
| | Visit number | | | | |
| | 22, n2^{a} | 1999 | 2999 | | |
| | Day^{e} | | | | |
| | | ≤28 | 29-56 | | |
| | | days | days | | |
| | | after | after | | |
| | | last | last | Every | Every |
| | 15^{a} | dose | dose | 12 weeks | 12 weeks |
| Screening procedures | | | | | |
| Informed consent | | | | | |
| Inclusion/exclusion criteria | | | | | |
| Demographic data and | | | | | |
| baseline characteristics | | | | | |
| Demographic data | | | | | |
| GEP-NET medical and | | | | | |
| treatment history | | | | | |
| (including mitotic rate | | | | | |
| and Ki-67 index) | | | | | |
| Medical history | | | | | |
| Prior medications | | | | | |
| ECOG performance status | | X | | | |
| Height, BMI | | | | | |
| Weight | | X | | | |
| Randomization procedures | | | | | |
| Randomization | | | | | |
| IMP administration | | | | | |
| CAM2029 administration^{g} | X | | | | |
| Comparator administration | | | | | |
| NIMP administration | | | | | |
| Octreotide IR (rescue | X | X | | | |
| medication)^{h} | | | | | |
| Imaging | | | | | |
| Somatostatin-receptor | | | | | |
| imaging | | | | | |
| FDG-PETⁱ | | | | | |
| Tumor evaluation | Every 12 weeks from randomization until disease progression is confirmed by the BIRC | | | | |
| Gallbladder ultrasound^{k} | | | | | |
| Pharmacokinetics | | | | | |
| Plasma samples for octreotide | | | | | |
| measurement (pre-dose)^{l} | | | | | |
| Plasma samples for octreotide | | | | | |
| measurement (post-dose)^{l} | | | | | |
| Cardiac assessments | | | | | |
| ECG (pre-dose) | | X | | | |
| ECG (24 hours after injection) | | | | | |
| Laboratory assessments | | | | | |
| Hematology, biochemistry, | | X | | | |
| thyroid testing, HbA1c | | | | | |
| Coagulation | | | | | |
| Hepatitis (B, C) and HIV | | | | | |
| Pregnancy serum test | | | | | |
| Pregnancy urine test | | X | | | |
| Immunogenicityⁿ | | X | | | |
| Other safety assessments | | | | | |
| Physical examination | | X | | | |
| Vital signs | | X | | | |
| AEs | X | X | X^{o} | | |
| Concomitant medications and | X | X | X^{p} | | |
| procedures | | | | | |
| PROs | | | | | |
| EORTC QLQ-C30 | Day 1, every 12 weeks and at End of Treatment, until start of a next-line therapy (if applicable) | | | | |
| QLQ-GINET21 | Day 1, every 12 weeks and at End of Treatment, until start of a next-line therapy (if applicable) | | | | |
| SF-36 | Day 1, every 12 weeks and at End of Treatment, until start of a next-line therapy (if applicable) | | | | |
| PGI-S | Day 1, every 12 weeks and at End of Treatment, until start of a next-line therapy (if applicable) | | | | |
| TSQM | Every 12 weeks and at End of Treatment, until | | | | |
| | start of a next-line therapy (if applicable) | | | | |
| Patient exit interview | | X^{q} | | | |
| Other assessments | | | | | |
| Recording of hospitalizations | | X | | | |
| Feasibility of self- or partner- | At first 3 times of self/partner-administration, | | | | |
| administration of CAM2029^{r} | then collected only if unsuccessful | | | | |
| Recording of reasons for | X | | | | |
| withdrawal of self- or partner- | | | | | |
| administration (as applicable)^{r} | | | | | |
| Discontinuation | | | | | |
| Recording of next-line therapy | | X | X | X | X |
| since discontinuation of IMP | | | | | |
| Survival Follow-up | | | | | X |

^{a}The administration on Day 15 of each month is only applicable for patients randomized to the CAM2029 treatment group. Starting with the second injection on Day 15 in Month 2 (M2D15), patients may self- or partner-administer CAM2029 at home on Day 15 of each month (except for M3D15, which is a mandatory PK sample visit). If the patients are not administering CAM2029 at home, they may have the injections performed by a trial personnel, and the assessments outlined in italics in the table will be performed
^{b}The Safety Follow-up may be performed as a visit to the clinic or as a telephone call
^{c}The Efficacy Follow-up is only applicable for patients who discontinue IMP treatment due to other reasons than PD
^{d}The visits on Day 8 and Day 15 of Month 3 (M3D8 and M3D15) are mandatory PK sampling visits for patients who receive CAM2029 or octreotide LAR. The assessments outlined in italics will be performed for these patients. Patients who receive lanreotide ATG do not need to perform the visits on M3D8 and M3D15
^{e}Visit windows: for screening assessment see footnote f, for all other visits after baseline, there is a window of ±3 days for assessments except for radiological assessments where ±7 days is allowed
^{f}Screening Visit assessments should occur within 28 days before randomization, except for assessments marked with ^{f}, which should occur within 7 days before randomization
^{g}CAM2029 may be self- or partner-administered or the administrations may be performed by the trial personnel. The injections will be given SC in the abdomen or thigh. Home administration of CAM2029 may be performed from the second injection (Day 15) in Month 2
^{h}Octreotide IR may be self-injected for symptom rescue but must be avoided within 24 hours before each clinic visit
ⁱFDG-PET is strongly encouraged for Grade 3 patients
^{j}CT/MRI of the chest, abdomen and pelvis and whole-body bone scan (if applicable) must be performed within 28 days before randomization
^{k}Patients who have had a complete gallbladder removal do not need to undergo gallbladder ultrasounds
^{l}Plasma samples for octreotide measurements are only applicable for patients treated with CAM2029 or octreotide LAR
^{m}For patients receiving CAM2029, samples will be taken at 0.5, 2, 5, 24 and 168 h. For patients receiving octreotide LAR, only the sample at 24 h will be taken
ⁿEvaluation of anti-octreotide antibodies in patients receiving treatment with CAM2029 or octreotide LAR
^{o}Follow-up of ongoing AEs and recording of new SAEs assessed as related to the IMP
^{p}Only concomitant medications associated with SAEs
^{q}The patient exit interview will be performed approximately 1 week from the End-of-treatment Visit as a phone interview, regardless if the patients proceed to treatment with CAM2029 once weekly in the Extension Treatment Period or continue directly to the Safety Follow-up
^{r}Only applicable for patients receiving CAM2029
AE: adverse event;
ATG: autogel;
BMI: body mass index;
CT: computed tomography;
D: Day;
ECG: electrocardiogram;
ECOG: Eastern Cooperative Oncology Group;
EORTC QLQ-C30: European Organization for Research and Treatment of Cancer's Core Quality of Life Questionnaire;
FDG: fluorodeoxyglucose;
GEP-NET: gastroenteropancreatic neuroendocrine tumors;
FU: Follow-up;
h: hour;
HbA1c: hemoglobin A1c;
HIV: human immunodeficiency virus;
IMP: investigational medicinal product;
IR: immediate release;
Ki-67: a nuclear antigen;
LAR: long-acting release;
M: Month;
MRI: magnetic resonance imaging
NIMP: non-investigational medicinal product;
PET: positron emission tomography;
PGI-S: Patient Global Impression of Severity;
PRO: patient reported outcome;
QLQ-GINET21: Quality of Life Questionnaire - Neuroendocrine Carcinoid Module;
QTcF: QTc interval corrected by Friderica's formula;
SAE: serious adverse event;
SF-36: Short Form-36;
TSQM: Treatment Satisfaction Questionnaire for Medication

TABLE 6
Schedule of trial procedures and assessments for patients participating in the optional Open-label Extension Treatment Period

| | Trial Period | | | | | | | |
|---|---|---|---|---|---|---|---|---|
| | | | | | End-of- | Post-treatment | | |
| | Open-label Extension Treatment Period (optional)^{a} | | | | extension- | FU | | |
| | Month | Month | Month E2 | Month E2 | treatment | Safety | Efficacy | Survival |
| | E1^{b} | E1 | to En^{c} | to En^{d} | Visit | FU^{e} | FU^{f} | FU |
| | Visit number | | | | | | | |
| | | E12, | | E22, E23, | | | | |
| | | E13, | | E24 to En2, | | | | |
| | E11 | E14 | E21 to En1 | En3, En4 | E1999 | E2999 | | |
| | Day^{g} | | | | | | | |
| | | | | | ≤28 | 29-56 | | |
| | | | | | days after | days after | Every | Every |
| | 1 | 8, 15, 22^{h} | 1 | 8, 15, 22 | last dose | last dose | 12 weeks | 12 weeks |
| Eligibility procedures | | | | | | | | |
| Inclusion/exclusion criteria for | X | | | | | | | |
| the Extension Treatment Period | | | | | | | | |
| Demographic data and baseline | | | | | | | | |
| characteristics | | | | | | | | |
| ECOG performance status | X | | ME2, ME3 and thereafter | | | | | |
| | | | every 3 months | | | | | |
| Weight | X | | ME2, ME3 and thereafter | | | | | |
| | | | every 3 months | | | | | |
| IMP administration | | | | | | | | |
| CAM2029 once weeklyⁱ | X | X | X | X | | | | |
| NIMP administration | | | | | | | | |
| Octreotide IR (rescue | X | X | X | X | X | | | |
| medication)^{j} | | | | | | | | |
| Imaging | | | | | | | | |
| Tumor evaluation | Every 12 weeks from randomization until new disease | | | | | | | |
| | progression is confirmed by the BIRC | | | | | | | |
| Gallbladder ultrasound^{k} | | | Every 6 months | | | | | |
| Pharmacokinetics | | | | | | | | |
| Plasma samples for octreotide | X | | ME2, ME3 | | | | | |
| measurement (pre-dose) | | | | | | | | |
| Plasma samples for octreotide | | | ME2, ME3 and ME6: | | | | | |
| measurement (post-dose) | | | 24 h ME9 to MEn: | | | | | |
| | | | 24 h if ECG is | | | | | |
| | | | assessed | | | | | |
| Cardiac assessments | | | | | | | | |
| ECG (pre-dose) | X | | ME2, ME3 | | X | | | |
| ECG (24 hours after injection) | | | ME2, ME3, ME6 and thereafter | | | | | |
| | | | every 3 months only if mean | | | | | |
| | | | QTcF ≥481 msec at any time | | | | | |
| | | | before ME6 | | | | | |
| Laboratory assessments | | | | | | | | |
| Hematology, biochemistry, | X | | ME2, ME3 and thereafter | | X | | | |
| thyroid testing, HbA1c | | | every 3 months | | | | | |
| Coagulation | X | | ME3 and thereafter | | | | | |
| | | | every 3 months | | | | | |
| Pregnancy urine test | X | | If clinically indicated | | X | | | |
| Immunogenicity | X (pre- | | Every 6 months (ME6, | | X | | | |
| | dose) | | ME12 etc.) (pre-dose) | | | | | |
| Other safety assessments | | | | | | | | |
| Physical examination | X | | ME2, ME3 and thereafter | | | | | |
| | | | every 3 months | | | | | |
| Vital signs | X | | ME2, ME3 and thereafter | | | | | |
| | | | every 3 months | | | | | |
| AEs | X | X | X | X | X | X^{l} | | |
| Concomitant medications and | X | X | X | X | X | X^{m} | | |
| procedures | | | | | | | | |
| PROs | | | | | | | | |
| EORTC QLQ-C30 | Every 12 weeks and at End of Extension Treatment, until start of a next-line therapy (if applicable) | | | | | | | |
| QLQ-GINET21 | Every 12 weeks and at End of Extension Treatment, until start of a next-line therapy (if applicable) | | | | | | | |
| SF-36 | Every 12 weeks and at End of Extension Treatment, until start of a next-line therapy (if applicable) | | | | | | | |
| PGI-S | Every 12 weeks and at End of Extension Treatment, until start of a next-line therapy (if applicable) | | | | | | | |
| TSQM | Every 12 weeks and at End of Extension Treatment, until start of a next-line therapy (if applicable) | | | | | | | |
| Other assessments | | | | | | | | |
| Recording of hospitalizations | X | | X | | X | | | |
| Feasibility of self- or partner- | X | At first 3 times of self/partner-administration, | | | | | | |
| administrationⁿ | | then collected only if unsuccessful | | | | | | |
| Recording of reasons for | X | X | X | X | | | | |
| withdrawal of self- or partner- | | | | | | | | |
| administration (as applicable)ⁿ | | | | | | | | |
| Discontinuation | | | | | | | | |
| Recording of next-line therapy | | | | | X | X | X | X |
| since discontinuation of IMP | | | | | | | | |
| Survival Follow-up | | | | | | | | X |

^{a}Optional treatment with CAM2029 20 mg once weekly for patients in both treatment groups who showed PD during treatment with the IMP (CAM2029/comparator) in the Randomized Treatment Period
^{b}The Day 1 Visit of Month 1 in the Extension Treatment Period may be the same as the End-of-treatment Visit in the Randomized Treatment Period. If this is the case, assessments that were performed at the End-of-treatment Visit do not need to be repeated
^{c}Patients should visit the clinic to perform the Day 1 visit of each Month during the first 3 months. Thereafter, patients should visit the clinic on Day 1 every 3 months (i.e. Month 6, Month 9 etc.)
^{d}Patients may self- or partner-administer CAM2029 at home on Days 8, 15 and 22 of each month. If the patients are not self- or partner-administering CAM2029 at home, they may have the injections performed by a trial personnel and the assessments outlined in italics in the table will be performed
^{e}The Safety Follow-up may be performed as a visit to the clinic or as a telephone call
^{f}The Efficacy Follow-up is only applicable for patients who discontinue CAM2029 treatment due to other reasons than PD
^{g}There is a window of ±2 days for assessments except for radiological assessments where ±7 days is allowed
^{h}The visits on Day 8 and Day 15 of Month 1 are mandatory visits for patients who received octreotide LAR or lanreotide ATG in the Randomized Treatment Period. These patients may self- or partner-administer CAM2029 or have the injections performed by a trial personnel. The first self- or partner-injection should preferably be made on Day 1 of Month 1
ⁱCAM2029 may be self- or partner-administered or the administrations may be performed by trial personnel. The injections will be given SC in the abdomen or thigh. For patients who already self- or partner-administered CAM2029 in the Randomized Treatment Period, home administration of CAM2029 may be performed from the second injection (Day 8) in Month 1. For patients who received octreotide LAR or lanreotide ATG in the Randomized Treatment Period, home administration of CAM2029 may be performed from the fourth injection (Day 22) in Month 1
^{j}Octreotide IR may be self-injected for symptom rescue but must be avoided within 24 hours before each clinic visit
^{k}Patients who have had a complete gallbladder removal do not need to undergo gallbladder ultrasounds
^{l}Follow-up of ongoing AEs and recording of new SAEs assessed as related to CAM2029
^{m}Only concomitant medication associated with SAEs
ⁿOnly applicable for patients who did not self- or partner-administer CAM2029 in the Randomized Treatment Period
AE: adverse event;
ATG: autogel;
BIRC: Blinded Independent Review Committee;
E: Extension;
ECG: electrocardiogram;
ECOG: Eastern Cooperative Oncology Group;
EORTC QLQ-C30: European Organization for Research and Treatment of Cancer's Core Quality of Life Questionnaire;
FU: Follow-up;
h: hour;
HbA1c: hemoglobin A1c;
IMP: investigational medicinal product;
IR: immediate release;
LAR: long-acting release;
ME: Month in Extension Period;
NIMP: non-investigational medicinal product;
PD: progressive disease;
PGI-S: patient Global Impression of Severity;
PRO: patient-reported outcome;
QLQ-GINET21: Quality of Life Questionnaire - Neuroendocrine Carcinoid Module;
QTcF: QTc interval corrected by Fridericas's formula;
SAE: serious adverse event;
SC: subcutaneously;
SF-36: Short Form-36;
TSQM: Treatment Satisfaction Questionnaire for Medication

### 7.2 Screening and Baseline Procedures and Assessments

All screening evaluations must be completed and reviewed to confirm that potential participants meet all eligibility criteria in Section [000331] and Section [000334]. The Investigator will maintain a screening log to record details of all screened participants and to confirm eligibility or record reasons for screening failure, as applicable.

For patients who participate in the Extension Treatment Period, the eligibility criteria in Section [000336] and Section [000338] need to be confirmed on Day 1 of the period.

### 7.2.1 Informed Consent

The Investigator or designated trial personnel will explain the nature of the trial and its risks and benefits to the patient. The patient must voluntarily provide written informed consent on the applicable ICF at screening, before any trial-related procedures are performed. The patient's medical records must document that the consent process has been completed and that written informed consent has been obtained from the patient before initiation of any trial-specific procedures. Documentation that the patient was given adequate time to ask the Investigator (or designee) questions about their participation in the trial and that a signed and dated copy of the ICF was provided to the patient should also be included in the medical records or clinical chart.

### 7.2.2 Demographics

Age, sex, race, and ethnicity will be recorded at screening.

### 7.2.3 GEP-NET Medical and Treatment History

Data on GEP-NET will include diagnosis and extent of cancer (including staging at trial entry), mitotic rate, and Ki-67 index.

A complete treatment history for GEP-NET will be recorded, including prior anti-neoplastic therapies as treatment for cancer and prior embolization or ablative interventions.

### 7.2.4 Medical History

Relevant other medical history, including surgical procedures, within 5 years before screening and any clinically significant medical history more than 5 years before screening will be collected based on available medical records and patient interview.

### 7.2.5 Prior Medications

Medications (prescription and non-prescription, herbal medications/natural health products, or investigational drugs) taken by the patient during the 3 months before screening will be recorded in the source documentation as medication history. The Investigator will determine if the prior medication(s) affect the patient's eligibility to participate in the trial.

### 7.2.6 Physical Examination

A physical examination including all major body systems (general appearance, skin, neck, including thyroid, eyes, ears, nose, throat, lungs, heart, abdomen, back, lymph nodes, extremities and nervous system) will be performed at screening and at the time points indicated in Table 5 and Table 6. Clinically significant findings that were present before screening must be included on the Medical History eCRF page. Clinically significant findings that begin or worsen after screening must be recorded on the AE page of the eCRF.

Height, weight, and BMI will be measured/calculated at screening. Weight will also be measured at the time points indicated in Table 5 and Table 6.

### 7.2.7 Somatostatin-receptor Imaging

Somatostatin-receptor imaging will be performed in conjunction with the screening using any imaging modality available, e.g. octreotide scintigraphy or [⁶⁸Ga]-DOTATATE PET/CT. Any preexisting assessment done as part of standard of care within 3 months prior to randomization can be used. The date of assessment and information on somatostatin-receptor expression will be collected in the eCRF.

In addition, for patients with well-differentiated Grade 3 NET, FDG-PET is strongly encouraged. If FDG-PET is performed, the results must show that FDG avid areas of disease also are avid on somatostatin-receptor imaging.

### 7.2.8 Contraceptive Requirements

Women of childbearing potential must agree to use an acceptable method of birth control as defined in the ICF from screening to the Safety Follow-up Visit and must agree to be tested for pregnancy. Acceptable method(s) of birth control include the following:
Total abstinence (when this is in line with the preferred and usual lifestyle of the patient). Periodic abstinence (e.g. calendar, ovulation, symptothermal, and post-ovulation methods) and withdrawal are not acceptable methods of contraception
Female sterilization (has had surgical bilateral oophorectomy with or without hysterectomy, total hysterectomy, or tubal ligation) at least 6 weeks before screening. In the case of oophorectomy alone, the reproductive status of the woman must be confirmed by follow-up hormone level assessment
Male sterilization (at least 6 months before screening). The vasectomized male partner should be the sole partner for that patient
Barrier methods of contraception: female condom with or without spermicide or cap, diaphragm, or sponge with spermicide. Simultaneous use of male and female condoms with or without any other contraception method is not permitted
Use of oral (progestin-only), injected, or implanted hormonal methods of contraception or placement of an intrauterine device or intrauterine system, or other forms of hormonal contraception that have comparable efficacy (failure rate <1%), e.g. hormone vaginal ring or transdermal hormone contraception

In case of use of oral contraception, the woman should have been stable on the same pill for a minimum of 3 months before starting IMP treatment.

Women are considered post-menopausal and not of childbearing potential if they have had 12 months of natural (spontaneous) amenorrhea with an appropriate clinical profile (i.e. appropriate age, history of vasomotor symptoms) or have had surgical bilateral oophorectomy (with or without hysterectomy), or tubal ligation at least 6 weeks before screening. In the case of oophorectomy alone, the woman is considered not to be of childbearing potential only when her reproductive status has been confirmed by follow-up hormone level assessment.

### 7.3 Efficacy Assessments

### 7.3.1 Imaging Tumor Assessments

Local site Investigator/radiology assessment based on RECIST 1.1 will be used to determine patient eligibility at screening. Tumor response will be assessed locally and centrally based on RECIST 1.1 (36). The process for image collection and transmission to the central imaging core laboratory is described in an imaging manual. The central review by the BIRC is described in an imaging review charter.

### 7.3.1.1 Tumor Imaging

Tumor imaging assessments should comprise CT (strongly preferred) or MRI of the chest, abdomen, and pelvis. MRI should primarily be used when CT is contraindicated. Chest X-ray may be used for assessment of lung metastases in exceptional cases and only after the Sponsor's confirmation. The same imaging technique regarding modality and contrast should be used for a patient throughout the trial. If somatostatin-receptor imaging is performed using scintigraphy, a whole-body bone scan (alternatively, a whole-body MRI) must be acquired at screening to assess bone metastases. Patients with bone metastases at baseline will undergo further assessments of bone lesions performed at protocol-scheduled time points for tumor assessments and as per institutional practice.

### 7.3.1.2 Tumor Assessment

For all patients, the first tumor imaging assessment will be performed during the screening period. Any CT, MRI or whole-body bone scan assessments already completed during the regular work-up of the patient within 28 days prior to randomization, including before signing the ICF at screening, can be considered as the baseline images for this trial, if they fulfil the imaging requirements.

Patients will have tumor imaging assessments performed every 12 weeks as per the procedures in Table 5, Table 6 and Table 7. The 12-week interval should be respected even if treatment is temporarily withheld. After baseline, all assessments should be performed within ±7 days of the scheduled day of assessment. All trial imaging assessments will be performed until PD is verified by the BIRC (for details see Section [000485]).

If a patient discontinues IMP treatment for reasons other than radiologically documented PD, a tumor imaging should be performed at the End-of-treatment/End-of-extension-treatment Visit unless a CT/MRI for tumor measurement was performed within the previous 21 days. Efficacy assessments should continue at the scheduled visits as per Table 5, Table 6 and Table 7.

All scheduled images for all patients must be submitted to the central imaging core laboratory. Additional imaging (including other modalities) obtained at unscheduled time points to determine disease progression, as well as imaging obtained for other reasons, but which captures radiologic progression, must also be submitted to the central imaging core laboratory.

TABLE 7
Imaging Assessment Plan

| | | Randomized Treatment | End of |
|---|---|---|---|
| | | Period, Extension | Treatment/End of |
| | Screening Period: | Treatment Period, and | Extension |
| Procedure | Day −28 to Day −1 | Follow-up Periods | Treatment^{a} |
| Somatostatin-receptor | Mandated | Not applicable | Not applicable |
| imaging | | | |
| FDG-PET | Strongly encouraged for patients | Not applicable | Not applicable |
| | with Grade 3 NET | | |
| Multiphasic CT or | Mandated^{b} | Every 12 weeks | Mandated |
| MRI (chest, abdomen, | | (±7 days) | |
| pelvis) | | | |
| Whole-body bone | Only if somatostatin-receptor | As clinically indicated | As clinically |
| scan | imaging is performed using | | indicated |
| | scintigraphy^{b} | | |
| Brain CT or MRI | Only if suspected brain | As clinically indicated | As clinically |
| | metastases | | indicated |
| Additional imaging | As clinically indicated | As clinically indicated | As clinically |
| | | | indicated |

CT: computed tomography;
FDG: fluorodeoxyglucose;
ICF: informed consent form;
IMP: investigational medicinal product;
MRI: magnetic resonance imaging;
NET: neuroendocrine tumors;
PD: progressive disease;
PET: positron emission tomography
^{a}Tumor evaluation at the End-of-treatment Visit is required for patients who discontinue IMP treatment before the first scheduled post-baseline tumor assessment (Week 12/Month 3). Tumor evaluation is also required for patients whose previous tumor assessment did not demonstrate PD and was done more than 21 days before the End-of-treatment/End-of-extension-treatment Visit
^{b}Any CT, MRI or whole-body bone scan assessments already completed during the regular work-up of the patient within 28 days prior to randomization, including before signing the ICF at screening, can be considered as the baseline images for this trial, as long as they fulfil the imaging requirements

### 7.3.2 Determination of Progressive Disease (PFS)

PFS is defined as the time from the date of randomization to the date of the first documented disease progression as per RECIST 1.1 or death due to any cause, whichever occurs first, as assessed by the BIRC.

When the local Investigator first assesses radiologic evidence of PD, the BIRC at the central imaging core laboratory will verify the PD. The imaging core laboratory will expedite verification of radiologic PD to the trial site and to the Sponsor. Further details on the central confirmation of PD will be provided in an imaging review charter.

While the Investigator is waiting for the results of the BIRC review, the patient must continue to receive IMP treatment unless the Investigator believes that continuation would be detrimental to the patient's well-being.

If there is agreement between Investigator and BIRC in the determination of disease progression, the patient will discontinue the currently assigned IMP treatment.

If there is discordance between Investigator and BIRC in the determination of disease progression, the patient should continue receiving the IMP treatment unless there is medical need (i.e. rapid progression or clinical deterioration) for an immediate change in therapy. Patients will continue to have scans performed per protocol until the BIRC confirms PD.

### 7.3.3 Progression on Next-Line Therapy (PFS2)

PFS2 is defined as the time from date of randomization to the date of documented progression on next-line therapy (not including treatment with CAM2029 in the Extension Treatment Period) as per RESIST 1.1 or death from any cause, whichever comes first. For PFS2, the disease progression will be determined based on Investigator assessment of progression. For this purpose, subsequent anti-neoplastic therapies including start/end date, reason for discontinuation and date of disease progression will be captured in the eCRF. For the purpose of PFS2, it is not necessary to continue to collect tumor assessment data on subsequent anti-neoplastic therapies.

### 7.3.4 Progression on CAM2029 in the Extension Treatment Period (PFS-Ext)

PFS-ext is the time from date of randomization to the date of documented progression on CAM2029 20 mg once weekly as per RECIST 1.1 or death from any cause, whichever comes first, in the Extension Treatment Period. For PFS-ext, the disease progression will be determined based on BIRC assessment of progression.

### 7.3.5 Rescue Medication Use

Throughout the trial, the patients will record the dose(s), date(s), and exact time(s) of administration of octreotide IR in a patient diary. Administration of octreotide TR must be avoided within 24 hours before each clinic visit.

### 7.3.6 Patient-Reported Outcomes

### 7.3.6.1 Overview

Health-related quality of life and patient satisfaction questionnaires will be administered at the visits indicated in Table 5 and Table 6. The last assessment will be performed at the start of next-line therapy.

The PROs should be administered in the patient's local language. Only PROs validated in the local language and provided by the Sponsor will be used in this trial. The PROs should preferably be administered at the beginning of the trial visit before any tests or treatments are performed and before the patient receives results from any tests to avoid biasing the patient's perspective. Patients should be given enough space and time to complete all trial questionnaires and all questionnaires should be reviewed for completeness. If missing responses are noted, patients should be encouraged to complete any missing responses. Investigators should not encourage the patient to change responses reported in the questionnaires. If a patient refuses to complete a questionnaire, this should be documented in the source records. A patient's refusal to complete trial questionnaires is not a protocol deviation.

The Investigator must review and assess the completed questionnaires, including responses to the questions and any unsolicited comments written by the patient, for responses or comments that may indicate potential AEs or SAEs before performing the clinical examination. This review should be documented.

Interviews conducted in connection with the End-of-treatment Visit (patient exit interviews) will allow patients to describe their experiences with the trial regimen and activities. These interviews will give the patients an opportunity to articulate any anticipated or unanticipated changes (positive or negative) associated with treatment. Patients may describe the impact of treatment on their symptoms, the importance of symptom changes, and how treatment affected their functioning and activities. Additionally, patients can comment on other trial experiences, including the convenience of the trial regimen and/or trial visits and any challenging aspects of the treatment or participation in the trial.

If an AE or SAE is confirmed based on the responses to the questionnaires or during the patient exit interviews, the event should be recorded as instructed in Section [000536].

### 7.3.6.2 EORTC QLQ-C30

The European Organization for Research and Treatment of Cancer's Core Quality of Life Questionnaire (EORTC QLQ-C30, version 3.0) is recognized as a reliable and valid measure that is used to assess generic quality of life for cancer patients.

The EORTC QLQ-C30 contains 30 items and comprises both multi-item scales and single-item measures. These include 5 functional scales (physical, role, emotional, cognitive, and social functioning), 3 symptom scales (fatigue, nausea/vomiting, and pain), 6 single items (dyspnea, insomnia, appetite loss, constipation, diarrhea, and financial impact), and a global health status/quality of life scale (37).

The scores of all scales and single-item measures range from 0 to 100. A high scale score represents a higher response level. Thus, a high score for a functional scale represents a high/healthy level of functioning, a high score for the global health status/quality of life represents a high quality of life, but a high score for a symptom scale/item represents a high level of symptomatology/problems. All scoring will follow the scoring procedures defined by the EORTC Scoring Manual (38).

### 7.3.6.3 QLQ-GINET21

The EORTC Quality of Life Questionnaire—Neuroendocrine Carcinoid Module (QLQ-GINET21) is a disease-specific module that may be used with the EORTC QLQ-C30 and that has been developed for use in patients with GI-related NET, whose disease stages and treatments vary. This module comprises 21 questions assessing disease symptoms, side effects of treatment, body image, disease-related worries, social functioning, communication, and sexuality. This module has been developed according to the EORTC guidelines and has been shown to be a valid and responsive disease specific tool for assessing quality of life in NET of the gut, pancreas, and liver (39).

### 7.3.6.4 SF-36

The Short Form-36 (SF-36) is a widely used standardized instrument with strong psychometric properties (40). The SF-36 will be used to assess self-perceptions of general health functioning across multiple dimensions (including general, physical, and emotional/psychiatric functioning). Higher scores indicate better quality of life. The SF-36 can be used in clinical practice and research, health policy evaluations, and general population surveys. It comprises a physical component summary and a mental component summary, both for which a higher score indicates better health status.

### 7.3.6.5 PGI-S

The Patient Global Impression of Severity (PGI-S) assessment tool is included to assess the patients' overall perception of their condition. The PGI-S is a 1-item questionnaire using a balanced Likert scale that asks the patient to rate the severity of a specific condition on a single-state, 5-point, categorical scale. The PGI scales were modelled on the Clinical Global Impression scales developed in the 1970s (41). They are simple, direct and easy to use and can be tailored to specific conditions and disease parameters.

### 7.3.6.6 TSQM

The Treatment Satisfaction Questionnaire for Medication (TSQM) is a general PRO instrument designed to measure patient satisfaction with their medication (42).

The TSQM Version 1.4 will be used to compare the patients' satisfaction with CAM2029 or the comparator treatment during the trial. The questionnaire has 4 domains (effectiveness, side effects, convenience, and global satisfaction), each comprising 3 to 4 items. Responses are evaluated on a 5- to 7-point scale for each item. The calculation of domain scores will be done according to the instrument manual. All domain scores range from 0 to 100. Higher scores indicate a better outcome from the patient's perspective. The TSQM questionnaire and instrument manual will be included in the Manual of Procedures.

The TSQM questionnaire will be completed at the beginning of the scheduled trial visits according to Table 5 and Table 6, before the Investigator conducts any clinical assessment.

### 7.3.6.7 Patient Exit Interview

At designated trial sites, patients will undergo a telephone exit interview approximately 1 week following the End-of-treatment Visit to collect information regarding the patients' experience with the trial and trial treatment. The preferred target is to conduct interviews with 20 patients per treatment group.

The interview will be conducted via phone by an external designated qualitative trial interviewer from a designated vendor and will last for up to 60 minutes.

The trial personnel will collect contact information for the patient and notify the interviewer to schedule the interview. Any contact information will be stored in an encrypted database accessible only to relevant trial personnel and the external interviewers.

The interviews will be audio-recorded and transcribed in local language, de-identified, and thereafter translated to English.

If a patient describes a potential AE during the interview, the trial personnel must be informed as soon as possible. The Investigator will evaluate and report any AE/SAEs as applicable, see Section [000536].

Further details will be provided in a separate Interview Guide.

### 7.3.7 Resource Utilization (Hospitalizations)

If the patient is hospitalized during the period outlined in Table 5 and Table 6, the reason and the date(s) of admission and discharge from the hospital should be registered in the eCRF. Overnights stay due to protocol procedures (such as PK sampling) are not considered hospitalizations.

### 7.3.8 ECOG Performance Status

The performance status of the patient will be assessed according to the ECOG performance status scale in Table 8 (43) at the time points outlined in Table 5 and Table 6.

TABLE 8
ECOG performance status

| Grade | ECOG status |
|---|---|
| 0 | Fully active, able to carry on all pre-disease |
| | performance without restriction |
| 1 | Restricted in physically strenuous activity |
| | but ambulatory and able to carry out work of a |
| | light or sedentary nature, e.g. light housework, office work |
| 2 | Ambulatory and capable of all self-care but unable |
| | to carry out any work activities. Up and about |
| | more than 50% of waking hours |
| 3 | Capable of only limited self-care, confined to bed |
| | or chair more than 50% of waking hours |
| 4 | Completely disabled. Cannot carry on any self-care. |
| | Totally confined to bed or chair |
| 5 | Dead |

ECOG: Eastern Cooperative Oncology Group
Source: Oken et al (43)

### 7.4 Pharmacokinetic Assessments

### 7.4.1 Blood Collection and Handling

Blood samples for evaluation of plasma concentrations of octreotide will be collected from all patients who receive CAM2029 or octreotide LAR. Blood sampling will be performed at the time points indicated in Table 5 and Table 6.

All blood samples for analyses of octreotide will be taken by either direct venipuncture or indwelling cannula inserted in a forearm vein. Blood samples (2.5 mL) will be collected to yield 1 mL plasma for analysis of octreotide plasma concentration.

The date and exact time of dosing, as well as the date and exact time of blood sampling, must be recorded on the eCRF. Detailed instructions for the collection, handling, and shipment of blood samples will be provided separately.

### 7.4.2 Analytical Method

Plasma concentrations of octreotide will be measured using a validated liquid chromatography-tandem mass spectrometry assay with a lower limit of quantification of approximately 0.0286 ng/mL.

### 7.5 Safety Assessments

### 7.5.1 Adverse Events and Serious Adverse Events

### 7.5.1.1 Adverse Event Definitions

An AE (synonym: adverse experience) is defined as any untoward medical occurrence associated with the use of a drug in humans, in a patient or clinical trial subject administered a trial treatment and which does not necessarily have a causal relationship with this treatment (i.e. whether or not considered drug-related). An AE can, therefore, be any unfavorable and unintended sign (e.g. an abnormal laboratory finding), symptom, or disease temporally associated with the use of a trial treatment, whether or not considered related to the trial treatment. Patients will be instructed to contact the Investigator at any time after enrollment if any symptoms develop.

An ADR is any untoward and unintended response to a trial treatment assessed as related to any dose administered. AEs or SAEs assigned a causality assessment by the Investigator of “probably related” or “possibly related” will be considered by the Sponsor to be related for the purpose of defining ADRs and thereby also expedited reporting.

An AE is considered “unexpected” if the nature, severity, or outcome is not consistent with the reference safety information.

An SAE is any untoward medical occurrence that at any dose:
Results in death
Is life-threatening (an event in which the patient was at risk of death at the time of the event; it does not refer to an event that hypothetically might have caused death if it were more severe)
Requires inpatient hospitalization or prolongation of existing hospitalization*
Results in persistent or significant disability or incapacity
Consists of a congenital anomaly or birth defect
Is another medically important event
   Important medical events that may not be immediately life-threatening or result in death or hospitalization but may jeopardize the patient or may require intervention to prevent one of the other outcomes listed in the definition above should also be considered serious. This is based on the medical and scientific judgment of the Investigator
      *Exemptions may be made when hospitalization is due to:
Routine treatment or monitoring of the investigated indication, not associated with any deterioration in the condition
Elective or pre-planned treatment for a pre-existing condition unrelated to the indication under investigation that did not worsen
Admission to a hospital or other institution for general care, not associated with any deterioration in the condition
Disease progression/fluctuation

### Other Reportable Information

Certain information, although not considered an SAE, must be recorded, reported and followed up as indicated for an SAE (see Section [000551]), including the following:
Pregnancy during exposure to a trial treatment. If a pregnancy is confirmed, the IMP must be discontinued immediately. Information about pregnancy exposure includes the entire course of pregnancy and delivery and perinatal and neonatal outcomes, even if there are no abnormal findings. Both maternal and paternal exposures are considered other reportable information. For exposure involving the female partner of a male patient, the necessary information must be collected from the patient while respecting the confidentiality of the partner
Lactation exposure to an IMP with or without an AE
Overdose of an IMP with or without an AE
Inadvertent or accidental exposure to an IMP with or without an AE

### 7.5.1.2 Eliciting, Documenting, and Reporting of Adverse Events

The Investigator is responsible for ensuring that all AEs and SAEs are recorded on the AE page of the eCRF and reported to the Sponsor. AEs will be assessed from the time of signing the ICF until the Safety Follow-up Visit. If there is any doubt as to whether a clinical observation is an AE, the event should be reported.

Abnormal laboratory values shall be recorded as AEs if assessed as clinically significant by the Investigator. Laboratory abnormalities that are associated with an already reported medical condition will not be reported as separate AEs but will be used to assess the associated medical condition (e.g. increased neutrophil levels in case of reported infection or increased glucose in a patient with diabetes).

At every visit, patients will be asked a standard question to elicit any medically related changes in their well-being. They will also be asked if they have been hospitalized, have had any accidents, have used any new medications, or have changed concomitant medication regimens (both prescription and over-the-counter medications).

Information to be collected includes trial treatment, type of event, time of onset, dosage, Investigator-specified assessment of seriousness, severity, and relationship to trial treatment, and time of event resolution, as well as any required treatment or evaluations, and outcome. AEs resulting from concurrent illnesses, reactions to concurrent illnesses or reactions to concurrent medications must also be reported. All AEs should be followed until they have reached a final outcome or to the Safety Follow-up Visit, whichever comes first (see further details in Section [000562]).

The Medical Dictionary for Regulatory Activities (MedDRA) will be used to code all AEs.

Any medical condition that is present at the time that the patient is screened but does not deteriorate thereafter should not be reported as an AE. However, if it deteriorates at any time during the trial, it should be recorded as an AE. Disease progression (including fatal outcomes), if documented by use of appropriate method (RECIST 1.1), should not be reported as an AE/SAE.

### 7.5.1.3 Reporting of Serious Adverse Events

An AE that meets any of the SAE criteria (Section [000537]) must be reported to the Sponsor immediately (within 24 hours after the Investigator becomes aware of the occurrence of the SAE) to the contact details listed below, using the trial-specific SAE report form. The Investigator will assess whether there is a reasonable possibility that the trial treatment caused the SAE. Other reportable information as defined in Section [000537] should also be reported to the below contact immediately (within 24 hours after the Investigator becomes aware of the occurrence of the event).

The Sponsor is responsible for notifying the relevant regulatory authorities of any SAEs according to applicable legislation. The Investigator is responsible for notifying the Independent Ethics Committee (IEC)/Institutional Review Board (IRB) directly, as per IEC/IRB requirements.

### 7.5.1.4 Assessment of Severity

Severity is defined as a measure of the intensity of an AE or SAE and will be assessed according to the National Cancer Institute (NCI) Common Terminology Criteria for Adverse Events (CTCAE). If CTCAE grading does not exist for an AE, the severities of mild, moderate and severe, corresponding to Grades 1 to 3, will be used.

Changes in the severity of an AE should be documented to allow an assessment of the duration of the event at each level of intensity. An AE characterized as intermittent requires documentation of onset and duration of each episode.

### 7.5.1.5 Assessment of Outcome

The outcome of an AE or SAE will be classified using the following outcome ratings:
0=Unknown
1=Recovered/resolved
2=Recovering/resolving
3=Not recovered/not resolved/ongoing
4=Recovered/resolved with sequelae
5=Fatal

### 7.5.1.6 Assessment of Causality

The Investigator will assess the relationship to IMP treatment for all AEs and SAEs. The relationship will be characterized using the following causality ratings:
Probably related: An AE with a reasonable time sequence to the administration of the IMP, unlikely to be attributed to concurrent disease or other drugs or chemicals, and that follows a clinically reasonable response on withdrawal (de-challenge). Re-challenge information is not required to fulfill this definition
Possibly related: An AE with a reasonable time sequence to the administration of the IMP, but that could also be explained by concurrent disease or other drugs or chemicals. Information on drug withdrawal may be lacking or unclear
Not related: An AE with a temporal relationship to drug administration that makes a causal relationship improbable, or in which other drugs, chemicals, or underlying disease provide plausible explanations. There is no reasonable possibility that the event was caused by the trial treatment
Not applicable: This assessment can be used, for example, in cases where the patient did not receive any treatment with IMP

### 7.5.1.7 Follow-Up of Adverse Events

All AEs should be followed until they have reached a final outcome (see Section [000557]) or to the Safety Follow-up Visit, whichever comes first.

SAEs and Grade 3 (or severe) non-serious AEs that are assessed as “possibly related” or “probably related” to the IMP and that are ongoing at the Safety Follow-up Visit should be followed on a regular basis according to the Investigator's clinical judgment until a final outcome has been established.

The outcome “recovering” can be used as the final outcome for events that are stabilized (i.e. no further worsening is expected) and that the Investigator expects to resolve over time.

The outcome “not recovered” can be used as the final outcome for events that are not expected to resolve over time (e.g. cancer).

SAEs that are spontaneously reported by a patient to the Investigator after the Safety Follow-up Visit, and for which a reasonable possibility of a causal relationship to the IMP is assessed by the Investigator (“possibly” or “probably” related), should be reported to the Sponsor by the Investigator, regardless of the time that has elapsed (post-trial events).

### 7.5.2 Anticipated Risks and Safety Concerns of the Trial Drug

The most frequent ADRs reported during octreotide therapy include GI disorders, nervous system disorders, hepatobiliary disorders, and metabolism and nutritional disorders.

The most commonly reported ADRs in clinical trials with octreotide administration were diarrhea, abdominal pain, constipation, nausea, flatulence, headache, cholelithiasis, hyperglycemia and injection-site reactions. Other commonly reported ADRs were dizziness, biliary sludge, thyroid dysfunction (e.g., decreased thyroid stimulating hormone, decreased total T4, and decreased free T4), loose stools, impaired glucose tolerance, vomiting, asthenia, and hypoglycemia (30).

Based on the known safety profile of lanreotide, the risks and safety concerns are not anticipated to be essentially different to those of octreotide (34).

### 7.5.2.1 Cardiovascular System

Bradycardia has been reported in patients treated with octreotide with frequency ranging between 1% and 10% (30). Therefore, dose adjustment of medicinal products such as beta blockers, calcium channel blockers, or agents to control fluid and electrolyte balance may be necessary during octreotide administration.

### 7.5.2.2 Gallbladder and Related Disorders

Octreotide inhibits secretion of cholecystokinin, resulting in reduced contractility of the gallbladder and an increased risk of sludge and stone formation. Development of gallstones has been reported in 15 to 30% of long-term recipients of Sandostatin IR. The prevalence of gallstones in the general population (aged 40 to 60 years) is about 5 to 20% (30). If gallstones do occur, they are usually asymptomatic; symptomatic stones should be treated either by dissolution therapy with bile acids or by surgery.

### 7.5.2.3 Glucose Metabolism

Because of its inhibitory action on growth hormone, glucagon, and insulin release, octreotide may affect glucose regulation. Post-prandial glucose tolerance may be impaired. In some instances, persistent hyperglycemia may be induced as a result of chronic administration. Hypoglycemia has also been reported (30).

In patients with concomitant type 1 diabetes mellitus, octreotide can affect glucose regulation, and insulin requirements may be reduced. In non-diabetics and type 2 diabetics with partially intact insulin reserves, Sandostatin IR administration resulted in increases in post-prandial glycemia in some subjects.

### 7.5.3 Clinical Safety Laboratory Assessments

Clinical safety laboratory assessments will be performed at screening and during the trial at the time points outlined in Table 5 and Table 6.

A central laboratory will be used to analyze all laboratory evaluations. Details on the collections, shipment of samples, and reporting of results by the central laboratory will be provided separately. The results of the clinical safety laboratory assessments at screening must be reviewed before enrollment to assess the patient's eligibility for the trial.

Laboratory values that are out of normal range must be evaluated for clinical significance. Clinically significant abnormalities present at screening should be reported on the Medical History eCRF page. Clinically significant findings must be discussed with the Medical Monitor before enrolling the patient in the trial.

New or worsened clinically significant findings occurring after screening must be recorded on the AE eCRF page.

Blood samples for laboratory assessments will be taken after the assessment of vital signs and ECG (as described below).

Locally required tests, e.g. for SARS-CoV-2, may be performed and analyzed at local laboratories. Such tests will not be part of clinical trial assessments.

The assessments in Table 9 will be performed:

TABLE 9
Clinical laboratory tests

| Test category | Test name |
|---|---|
| Hematology | Hematocrit, hemoglobin, mean corpuscular |
| | hemoglobin, mean corpuscular hemoglobin |
| | concentration, mean corpuscular volume, red |
| | blood cell morphology, platelets, red |
| | blood cells, WBC, WBC with differential |
| | (basophils, eosinophils, lymphocytes, monocytes, |
| | neutrophils) |
| Biochemistry | Albumin, ALP, ALT, AST, amylase, bicarbonate, |
| | calcium, chloride, creatinine, creatine kinase, |
| | GGT, lipase, plasma glucose, inorganic phosphorus, |
| | magnesium, potassium, sodium, total bilirubin |
| | (direct bilirubin and indirect bilirubin only in |
| | subjects/patients with total bilirubin >2 × |
| | ULN), total cholesterol, low-density lipoprotein, |
| | high-density lipoprotein, total protein, triglycerides, |
| | blood urea nitrogen or urea, uric acid |
| Urinalysis | Macroscopic panel (bilirubin, blood, glucose, ketones, |
| | WBC, pH, protein, specific gravity) |
| Coagulation | Prothrombin time or INR, activated partial |
| | thromboplastin time |
| Thyroid | T3 (free), T4 (free), TSH |
| Serology | HBsAg, HCV antibodies (PCR required for HCV |
| | antibody-positive subjects/patients), HIV virus |
| | and HIV antibodies |
| Immunogenicity | Anti-octreotide antibodies |
| Additional tests | HbA1c |

ALP: alkaline phosphatase;
ALT: alanine aminotransferase;
AST: aspartate aminotransferase;
GGT: gamma glutamyl transferase;
HbA1c: hemoglobin A1c;
HBsAg: hepatitis B surface antigen;
HCV: hepatitis C virus;
HIV: human immunodeficiency virus;
INR: international normalized ratio;
PCR: polymerase chain reaction;
TSH: thyroid stimulating hormone;
ULN: upper limit of normal;
WBC: white blood cells

### Immunogenicity Assessments

Blood samples for immunogenicity assessment of anti-drug antibodies for octreotide will be collected from patients treated with CAM2029 or octreotide LAR according to Table 5 and Table 6. The blood samples will be sent to a central laboratory. Detailed method descriptions of the immunogenicity assays will be provided separately.

### Pregnancy Tests

Women of childbearing potential will have a serum beta-human chorionic gonadotropin pregnancy test at screening. This will be performed by a central laboratory. The results of the pregnancy test at screening must be reviewed and confirmed to be negative before enrollment to assess the patient's eligibility for the trial. Moreover, urine pregnancy test will be performed at the other visits, as indicated in Table 5 and Table 6. Urine pregnancy tests will be performed by dipstick locally. A positive pregnancy test requires immediate interruption of IMP treatment until the pregnancy is confirmed via a serum pregnancy test performed by a central laboratory. If confirmed positive, the patient must be discontinued from the trial.

### 7.5.4 Vital Signs

Vital signs consist of body temperature, blood pressure (systolic and diastolic, mmHg), pulse rate (beats per minute), and respiratory rate (breaths/min) and will be collected at the time points outlined in Table 5 and Table 6, following a resting period of at least 3 minutes. More frequent examinations may be performed at the Investigator's discretion, if medically indicated. Vital signs will be measured before performing ECG and collecting blood samples.

Significant findings present at screening must be included on the Medical History eCRF page. Significant findings that begin or worsen after screening must be recorded on the AE page of the eCRF.

### 7.5.5 Electrocardiogram

ECGs will be recorded and readings will be transmitted to a selected ECG central laboratory for analysis. Triplicate 12-lead ECG (3 ECG recordings at approximately 2-minute intervals) will be performed at each scheduled time point. The combined QTcF values from triplicate ECGs will be averaged to provide a single value for each patient. ECGs will be recorded at screening and at the time points outlined in Table 5 and Table 6.

ECGs will be recorded after the patient has been resting in a supine position for at least 10 minutes. All ECGs should be recorded with the patient in the same physical position. ECGs will be recorded after vital signs and before blood samples for PK and clinical safety laboratory assessments.

The ECG assessments pre-dose and at the 24-hour time point have been selected to coincide with trough concentrations and near Cₘₐₓ of octreotide for CAM2029, based on PK results from trial HS-11-411. If at any visit QTcF≥481 msec is observed, the procedures for QT-prolongation management described in Section [000760], Appendix 1 must be considered.

All ECG assessments will initially be assessed by the Investigator/qualified physician for any findings that require immediate medical attention. All ECGs will also be read by an ECG central reader. The clinical significance of any ECG findings will be determined by the Investigator, including after the central reading result is available. However, only ECG results provided by the central reader will be recorded in the clinical database.

Clinically significant abnormalities present at screening should be reported on the Medical History eCRF page. Clinically significant findings must be discussed with the Sponsor before enrolling the patient in the trial. New or worsened clinically significant findings occurring after screening must be recorded on the AE page of the eCRF.

### 7.5.6 Gallbladder Ultrasound Examination

Gallbladder ultrasounds will be performed locally at visits indicated in Table 5 and Table 6. Patients with symptomatic cholelithiasis at screening will be excluded from participating in the trial (refer to Section [000334]). Information on the presence and location of gallstones, gallbladder sludge, and biliary dilatation will be recorded in the appropriate eCRF page.

Historical gallbladder ultrasound performed within 3 months before screening may be used as baseline measurement. If such a gallbladder ultrasound is available, the screening gallbladder ultrasound does not need to be performed.

Patients who have had a complete gallbladder removal do not need to undergo gallbladder ultrasounds.

### 7.6 Other Assessments—Feasibility of Self- or Partner-Administration of CAM2029

For patients receiving CAM2029, the patients and their partners will have the possibility to administer CAM2029 by themselves (after appropriate training and the option of simulated injection). The first self- or partner-administration should preferably be performed at the first injection. Qualified trial personnel will supervise the first 3 administrations and assess the patient's/partner's ability to successfully administer CAM2029 using a checklist based on assessments used in previous studies on SSAs (44, 45), see the Manual of Procedures.

The qualified trial personnel will train the patient or their partner, and the training is successful, a supervised self- or partner-administration according to the provided checklist can be performed. The patient or their partner may receive training in administration of CAM2029 for a maximum of 3 times (3 visits). The training attempts and outcomes will be collected as “successful” or “unsuccessful” on the appropriate eCRF page.

If the patient/partner is deemed as incapable to administer CAM2029, the trial personnel will continue the administration at the scheduled time points indicated in Table 5 and Table 6. If a patient or a partner is declared competent by the qualified trial personnel to successfully self- or partner-administer CAM2029, then he/she will have the possibility to continue to self- or partner-administer CAM2029 throughout the trial.

Any problem during the CAM2029 administration (technical or human-factor-related), and any reason why a patient decides to withdraw from self- or partner-administration of CAM2029 will be recorded in the eCRF.

### 7.7 Data Monitoring Committee

A DMC will be established for this trial. The DMC will include a minimum of 2 physicians with appropriate disease area qualifications and 1 statistician. The DMC will conduct periodic reviews of safety data from the trial and will continuously review Grade 3 ADRs and serious ADRs. The DMC will recommend actions to the Sponsor, as appropriate. The DMC will be informed to what extent the data and analyses provided to them have been quality controlled. Members of the DMC will not be involved in other trial-related tasks. The DMC procedures are described in the DMC Charter.

### 8 Statistical Considerations

### 8.1 Statistical and Analytical Plans

Complete details of the statistical analyses to be performed will be documented in a statistical analysis plan (SAP), which will be completed before the first patient is randomized in the trial. This document will include more details of the analysis populations, summary strategies, and any amendments to the proposed analyses, if necessary. Any changes to the SAP will be outlined in the final clinical trial report.

### 8.2 Determination of Sample Size

A one-sided log rank test with an overall sample size of 280 patients (140 in the CAM2029 treatment group and 140 in the comparator treatment group) will achieve at least 85% power at a 0.025 (one-sided) significance level to detect a hazard ratio of 0.65 when the comparator group median time to progression or death is 18 months. Under the same assumptions, an observed hazard ratio of 0.755 or less should give a p-value <0.025 (one-sided). The Randomized Treatment Period of the trial is planned to run for 48 months, of which patient accrual (entry) occurs in the first 18 months. The accrual pattern across time periods is assumed to be uniform. Given the assumptions, 194.3 events are needed. The event rate will be monitored during the trial. These calculations are neither adjusted for potential loss to follow up, nor for cross-over between treatments.

With a loss of at most 10% for 36 months, and similar in the 2 treatment groups, a total of 292 patients are needed. For a 15% loss to follow up over 3 years, 302 patients are needed. The sample size calculations were performed in PASS 16.

The trial will also evaluate the treatment effect on overall survival. Assuming a median time of 80 months to death in the comparator group, the expected number of events at 48 (72) months is approximately 77 (114). With a true hazard ratio of 0.75 favoring treatment with CAM2029, the power is approximately 24% (33%) using a significance level of 0.025% one-sided. To control the overall significance level for the analysis of overall survival, a group sequential approach will be used where the treatment difference in overall survival will be tested with a stratified log rank test at 48 months, using a one-sided significance level of 0.72% and at 72 months, using a one-sided significance level of 2.26%.

### 8.3 General Considerations

The primary efficacy analysis will be based on the Randomized Treatment Period of the trial and will compare patients randomized to receive CAM2029 against participants randomized to receive the comparator product.

The primary estimand will be used for the analysis of the primary efficacy endpoint, PFS. It will be based on patients in the intent-to-treat (IIT), which is defined as all randomized patients who received at least 1 dose of IMP, analyzed according to their randomized treatment. The set of intercurrent events for this estimand consists of patients who withdraw from the trial prior to having met the primary efficacy endpoint. The intercurrent events will be handled using the treatment policy strategy and the potential absence of data following these patients' withdrawal will be treated as missing (i.e. counted as not having met the criteria of PFS).

Additional estimands will be specified for the primary efficacy endpoint to carry out sensitivity analyses for assessing the robustness of results. These sensitivity analyses will explore different methods for handling intercurrent events and different assumptions for missing data. Estimands will also be specified for the analysis of secondary endpoints. Full details will be provided in the SAP.

For overall survival, 2 analyses are planned; 1 at the end of the Randomized Treatment Period followed by the final analysis of overall survival at end of the Survival Follow-up.

Categorical variables will be summarized using frequency and percentages, where the denominator for calculation is the underlying analysis set population unless otherwise stated.

Continuous variables will be summarized with descriptive statistics, including number of available observations, mean, standard deviation, median, minimum and maximum, and quartiles where more appropriate.

Survival endpoints will be summarized by presenting the quartiles of the survival distribution and the survival rate at 12, 24, and 36 months in each treatment group, as well as the hazard ratio and 95% CI for the comparison between the treatment groups.

For all efficacy endpoints, descriptive measures and 95% CI for the difference between the 2 treatment groups (CAM2029 and comparator) will be presented, unless otherwise specified.

Safety analyses will be based on the safety analysis set. Safety parameters will be presented descriptively.

The statistical analyses will be performed using SAS® version 9.3 or later.

### 8.4 Patient Disposition

All patients screened and enrolled will be accounted for. All post-enrollment discontinuations will be summarized by time of and reason for discontinuation. Patients who are screened but not enrolled will be listed.

### 8.5 Protocol Deviations

Major protocol deviation criteria will be established before database lock.

### 8.6 Analysis Sets

### 8.6.1 Intention-to-Treat Analysis Set

The IIT analysis set comprises all patients who have been randomized to a treatment group. Analyses based on this population will group patients according to the treatment they were randomized to receive, regardless of actual treatment received.

The efficacy analyses will be based on the ITT analysis set.

### 8.6.2 Full Analysis Set

The full analysis set comprises all randomized patients in the IIT analysis set who received at least 1 dose of the randomized IMP.

### 8.6.3 Per Protocol Analysis Set

The per protocol analysis set is defined as all patients in the IIT analysis set with no major protocol deviations that would impact the efficacy assessment. Detailed criteria defining this analysis set will be documented in the SAP.

### 8.6.4 Pharmacokinetic Analysis Set

The PK analysis set will include all enrolled patients who were administered CAM2029 or octreotide LAR and for whom at least 1 post-dose plasma octreotide concentration result is available after the IMP administration.

The PK analysis set will be used for analysis of PK data.

### 8.6.5 Safety Analysis Set

The safety analysis set comprises all patients who were administered at least 1 dose of IMP. Analyses based on this population will group patients according to the actual treatment the patients received.

### 8.7 Trial Population

### 8.7.1 Demographics and Other Baseline Characteristics

All relevant demographic and baseline characteristics will be summarized using descriptive statistics.

The number and reasons for withdrawals and discontinuations will be listed and tabulated by treatment.

### 8.7.2 Medical History and Disease Characteristics

Medical history (recorded at screening) will be coded using MedDRA and data will be listed and presented descriptively. Disease characteristics will also be listed and presented descriptively.

### 8.7.3 Prior and Concomitant Medications and Treatments

Prior and concomitant medications will be summarized separately by Anatomical Therapeutic Chemical (ATC) classification first level (alphabetically) and ATC classification second level (in decreasing order of frequency).

Treatment history for GEP-NET will be listed and summarized descriptively.

### 8.8 Efficacy Endpoints and Analyses

### 8.8.1 Missing Values

The handling of missing data is presented in the sections describing the primary and secondary endpoints. Every effort will be made to collect all data for all patients in the trial. Data from patients who withdraw consent to participate in the trial will be included up to the date of their trial withdrawal.

Further details on the handling of missing data will be presented in the SAP.

### 8.8.2 Primary Efficacy Endpoints

The primary efficacy endpoint of the trial is PFS, which is defined as the time from the date of randomization to the date of the first documented disease progression as per RECIST 1.1 or death due to any cause, whichever occurs first, as assessed by the BIRC. The primary efficacy endpoint, PFS, will be analyzed based on the data from the ITT analysis set in the Randomized Treatment Period, according to treatment group.

### Definition of the Primary Estimand

The items that define the primary estimand are the following:
a) Population of interest: Patients with GEP-NET as defined by the inclusion/exclusion criteria in the trial
b) Variable (or endpoint) of interest: PFS, where the time point of progression or death will be used as a composite variable. Patients without progression and who are still alive will be censored at the end of the trial in the analysis
c) Specification of how intercurrent events are reflected in the scientific question of interest:
   Discontinuation: Patients who discontinue treatment with IMP will be analyzed by the treatment policy strategy, i.e. discontinuation will not lead to censoring in the analysis
   Missing values: If the status of the PFS cannot be determined at the end of the trial, the patient will be regarded as censored at the time of the last radiology assessment
   Rescue medication: Patients who use rescue medication will be analyzed by the treatment policy strategy, i.e. no adjustment will be made for rescue medication use
d) Population level summary for the variable: Hazard ratio and log rank test stratified by the randomization stratification factors

Thus, the proposed primary estimand is the entity defined to address the effect of treatment with CAM2029 or comparator product on reducing the risk of disease progression or death in patients with a diagnosis as defined by the inclusion and exclusion criteria.

### Statistical Analysis Methods

The following statistical hypotheses will be tested to address the primary efficacy objective:

H₀: H_{R}≥1 versus H_{A}: HR<1

where ‘HR’ is the PFS hazard ratio and a hazard ratio less than 1 indicates a better treatment effect with CAM2029 than for the comparator in reducing the instantaneous risk of experiencing PD or death. The primary efficacy analysis to test this hypothesis and compare the 2 treatment groups will be a stratified log rank test. The stratification will be based on the randomization stratification factors. In the primary analysis, PFS will be censored at the date of the last adequate tumor assessment if no PFS event is observed prior to the analysis cut-off date.

A stratified Cox regression model will be used to estimate the hazard ratio of PFS, along with the associated 95% CI. The stratification will be based on the randomization stratification factors and the censoring will be calculated as for the log rank test. If the number of patients in a stratification group is small, this stratification factor will not be used in the log rank test.

### Supportive and Sensitivity Analyses

Sensitivity analyses will be explored to assess the robustness of treatment effects for the primary efficacy endpoint, where different missing data mechanisms will be explored using various imputation approaches. Full details of the per protocol and sensitivity analyses will be specified in the SAP and documented prior to the randomization of the first patient into the trial.

### 8.8.3 Secondary Efficacy Endpoints

The analyses of secondary endpoints will be performed at the time of the final PFS analysis.

### 8.8.3.1 PFS Based on Local Investigator Assessment

PFS based on local Investigator assessments will be analyzed using a stratified log rank test and a stratified Cox model, with the same conventions as those of the primary efficacy analysis (see Section 8.8.2). The treatment effect will be summarized by the hazard ratio with its 95% CI.

### 8.8.3.2 Overall Survival

Overall survival is defined as the time from date of randomization to date of death due to any cause. If a patient is not known to have died, then overall survival will be censored at the last date the patient is known to be alive.

Overall survival will be analyzed using a stratified log rank test and a stratified Cox model with the same conventions as that of the primary efficacy analysis. The treatment effect will be summarized by the hazard ratio with its 95% CI.

An O'Brien Fleming stopping boundary (as implemented in PASS16) will be used for the analysis of overall survival. At the first analysis at the end of the Randomized Treatment Period, the observed p-value must be less than 0.0072 in order to conclude superior efficacy. If the trial continues to the final analysis approximately 72 months after the first randomization of a patient, the p-value that will be used to declare statistical significance at the final analysis will be 0.0226 (one-sided). By utilizing an O'Brien Fleming stopping boundary for efficacy, treatment with CAM2029 will be declared effective at interim only if overwhelming efficacy is demonstrated.

### 8.8.3.3 Overall Response Rate and Disease Control Rate

ORR is defined as the proportion of patients with best overall response of complete response (CR) or partial response (PR), as per BIRC according to RECIST 1.1. The ORR will be analyzed with Cochran-Mantel-Haenszel test stratified by the randomized stratification factors.

DCR is defined as the proportion of patients with a best overall response of CR, PR, or SD as per BIRC according to RECIST 1.1. DCR will be analyzed using similar model as for ORR.

### 8.8.3.4 Time to Tumor Response and Duration of Response

Time to tumor response is defined as the time from the date of randomization to the first documented response of either CR or PR as per BIRC according to RECIST 1.1 and will be analyzed in a similar model as for PFS.

Duration of response only applies to patients whose best overall response is CR or PR according to RECIST 1.1 based on tumor-response data per BIRC review. The start date is the date of first documented response of CR or PR (i.e. the start date of response, not the date when response was confirmed), and the end date is defined as the date of the first documented progression or death due to underlying cancer. Patients continuing without progression or death due to underlying cancer will be censored at the date of their last adequate tumor assessment. Duration of response will be summarized by descriptive statistics for all patients in the IIT analysis set with confirmed best overall response of CR or PR.

### 8.8.3.5 Rescue Medication Use

The average number of injections of rescue medication per month for each patient during trial, and the total dosage and dose intensity will be described by summary statistics.

### 8.8.3.6 Health-related Quality of Life

Change from baseline in the QLQ-GINET21, SF-36, and the global health status/quality of life scale score of the EORTC QLQ-C30 will be analyzed in a mixed model for repeated measures (MNIRM).

### 8.8.3.7 Patient Satisfaction

TSQM scores over time using all 4 domains of TSQM (effectiveness, side effects, convenience, and global satisfaction) will be analyzed in an MMRM with factors for treatment, visit, and their interaction.

### 8.8.4 Adjustments for Multiplicity

Two hypothesis tests will be performed for this trial. To control overall Type I error rate at 2.5% (one-sided), a stepwise closed testing procedure will be used.

Specifically, at the end of the Randomized Treatment Period (approximately at 48 months), the following ordered hypotheses will be tested for comparing the treatments:
Superiority for PFS as judged by the BIRC (primary endpoint)
Superiority for overall survival

If the primary analysis of PFS by BIRC is not statistically significant, then the secondary endpoints defined in the hierarchy testing strategy will not be tested. If the analysis of PFS based on local Investigator assessment is not statistically significant, the subsequent endpoint of overall survival will not be tested.

Both PFS endpoints will be tested separately at the 2.5% significance level (one-sided).

Overall survival will be tested in a 2-stage approach, and will, at the end of the Randomized Treatment Period, be tested at the 0.72% significance level (one-sided), i.e. p<0.0072 will be required to show improved survival rate. If p>0.0072, then the overall survival outcome at the end of the Survival Follow-up, at approximately 72 months from the randomization of the first patient, will be tested at the one-sided 2.26% significance level.

### 8.9 Pharmacokinetic Analyses

The PK analysis set will be used for all analyses in this section unless specified otherwise.

Descriptive statistics (n, m [number of non-zero concentrations], arithmetic mean, coefficient of variation percentage mean, standard deviation, median, geometric mean, coefficient of variation percentage geo-mean, minimum and maximum) for octreotide concentration will be presented at each scheduled time point by CAM2029 dose and for octreotide LAR.

Individual concentration-time profiles will be displayed graphically by CAM2029 dose and for octreotide LAR on the linear and semi-log view. In addition, the arithmetic mean (±standard deviation) concentration-time profiles will be displayed graphically on the linear and semi-log view. All individual plasma concentration data for CAM2029 and octreotide LAR will be listed.

PK/efficacy analyses: The relationship between octreotide exposure and PFS will be explored. Other relevant endpoints (such as tumor response) may also be considered.

PK/safety analyses: The relationship between octreotide exposure and QTcF change from baseline will be explored using a linear mixed effect model, including octreotide concentration as a fixed effect and patient as a random effect. Other safety endpoints may also be considered.

Plasma concentration data generated from this trial (PK analysis set) will be used together with data from other clinical trials in a population PK assessment. Patient demographics and baseline data (e.g. age, sex, race, ethnicity, body weight, BMI) and relevant laboratory assessments will be explored as covariates, if appropriate. The broad principles outlined in the FDA “Guidance for Industry: Population Pharmacokinetics” (48) will be followed during the population PK analysis along with any applicable internal Guidance and Standard Operating Procedures. The results from the population PK analysis will be presented in a separate report.

### 8.10 Safety Endpoints and Analyses

For all safety analyses, the safety analysis set will be used.

The overall observation period will be divided into 4 mutually exclusive segments:
1. Pre-treatment period: from the day of the patient's informed consent to the day before first dose of IMP
2. Randomized Treatment period: from day of first dose of IMP to Day 56 after last dose of IMP (i.e. Safety Follow-up) or to start of the Extension Treatment Period (if the patient proceeds to that period)
3. Extension Treatment Period: from day of first dose of CAM2029 20 mg once weekly to Day 56 after last dose (i.e. Safety Follow-up)
4. Post-treatment period: starting on Day 57 after last dose of IMP

### 8.10.1 Adverse Events

Summary tables for AEs will include only AEs that started or worsened after the first administration of IMP, i.e. the treatment-emergent AEs (TEAEs). An overview of all TEAEs including severity, relationship to the IMP, injection site AEs, SAEs, and AEs leading to withdrawal or death will be presented by treatment group, treatment, and overall.

TEAEs will be summarized by MedDRA system organ class and preferred term, displaying number of patients in the group, number, and percentage of patients having the AE, as well as the number of AEs. Furthermore, AEs will be summarized according to severity, relationship, outcome, and seriousness.

In addition, summary tables will be prepared for TEAEs with an incidence of at least 5% or lower threshold as applicable for reporting.

SAEs, AEs leading to withdrawal during the Treatment Periods, and injection site AEs will be listed and tabulated, if appropriate. All deaths (on-treatment and post-treatment) will be summarized.

All AEs, deaths, and SAEs will be listed and those collected during the pre- and post-treatment periods will be flagged.

Details of the analyses will be presented in the SAP.

### 8.10.2 Clinical Safety Laboratory Assessments

Grading of laboratory values will be assigned programmatically as per NCI CTCAE. The calculation of CTCAE grades will be based on the observed laboratory values only; clinical assessments will not be taken into account.

CTCAE Grade 0 will be assigned for all non-missing values not graded as 1 or higher.

For laboratory tests where grades are not defined by CTCAE, results will be categorized as low/normal/high based on laboratory normal ranges.

The following summaries will be generated separately for hematology and biochemistry tests:
Listing of all laboratory data with values flagged to show the corresponding CTCAE grades, if applicable, and the classifications relative to the laboratory normal ranges

For laboratory tests where grades are defined by CTCAE:
Worst post-baseline CTCAE grade (regardless of the baseline status). Each patient will be counted only once for the worst grade observed post-baseline
Shift tables using CTCAE grades to compare baseline to the worst on-treatment value

For laboratory tests where grades are not defined by CTCAE:
Shift tables using the low/normal/high/classification to compare baseline to the worst on-treatment value

All laboratory values will be presented in SI units.

In addition to the above tables and listings, other exploratory analyses, e.g. figures plotting time course of change in laboratory tests over time or box plots may be specified in the SAP.

### 8.10.3 Vital Signs

Vital signs data will be tabulated and listed. Clinically significant values will be flagged.

### 8.10.4 Electrocardiogram

Triplicate 12-lead ECGs, including ECG intervals, will be obtained for each patient during the trial. Data will be listed and summarized descriptively.

Categorical analysis of QT/QTcF interval data based on the number of patients meeting or exceeding predefined limits in terms of absolute QT/QTcF intervals or changes from baseline will be presented. In addition, a listing of these patients will be produced (by treatment group).

### 8.10.5 Physical Examination

Results from the physical examinations will be tabulated and listed. Significant findings will be flagged.

### 8.10.6 Gallbladder Imaging

Gallbladder data will be tabulated and listed.

### 8.11 Exploratory Endpoints and Analyses

### 8.11.1 PFS-Ext

PFS-ext is defined as the time from date of randomization to the date of documented disease progression as per RECIST 1.1 or death from any cause, whichever comes first, in the Extension Treatment Period. For PFS-ext, disease progression will be determined based on BIRC assessment of progression.

PFS-ext will be analyzed using a log rank test and a stratified Cox model, with the same conventions as those of the primary efficacy analysis of PFS. The treatment effect will be summarized by the hazard ratio with its 95% CI.

### 8.11.2 PFS2

PFS2 is the time from date of randomization to the date of documented progression on next-line therapy as per RECIST 1.1 or death from any cause. The documented progression on next-line therapy will be based on local Investigator assessment of PD.

PFS2 will be analyzed using a log rank test and a stratified Cox model, with the same conventions as those of the primary efficacy analysis of PFS (see Section [000659]). The treatment effect will be summarized by the hazard ratio with its 95% CI.

### 8.11.3 Resource Utilization

The number of patients who are hospitalized and the total number and length of hospitalizations will be described by summary statistics.

### 8.11.4 Patient Exit Interviews

Patient interview data will be analyzed by trained qualitative researchers. These analyses involve reviewing field notes, transcripts, and data-collection forms to identify patterns found in the interview data to and facilitate description of the themes and relative importance of concepts based on participants' experiences. Quantitative data, such as responses to close-ended questions or ranking exercises, are usually descriptive. Meaningful change will be established using concept elicitation and cognitive debriefing techniques.

### 8.11.5 PGI-S

The PGI-S data will be summarized using descriptive statistics and used in combination with other PROs included in trial and patient exit interviews to establish minimal important difference on the EORTC QLQ-C30.

### 8.11.6 ECOG Performance Status

Time to definitive deterioration in ECOG performance status is defined as the time from the date of randomization to the date when ECOG performance status has definitively deteriorated by at least 1 category compared with baseline. Deterioration is considered definitive if there is no subsequent improvement in ECOG performance status back to the baseline category or above. Patients will be censored if no definitive deterioration in ECOG performance status is observed before the first to occur between: (i) the analysis cut-off date, and (ii) the date when a next-line therapy is started. The censoring date will be the date of the last performance status assessment prior to cut-off/start of next-line therapy.

Time to definitive deterioration in ECOG performance status will be analyzed for the IIT analysis set according to the randomized treatment group and strata assigned at randomization. Kaplan-Meier curves, medians, and 95% CIs of the medians will be presented for each treatment group. The hazard ratio for the time to definitive deterioration in ECOG performance status will be calculated along with its 95% CI using a stratified Cox model.

### 8.11.7 Immunogenicity Assessments

The incidence and titer of drug-specific antibodies against octreotide will be correlated with safety, PK, and efficacy parameters collected across the trial, as applicable. In addition, the duration of previous octreotide treatment (if any) will be correlated with the presence and titer of the anti-drug antibodies at trial entry.

### 8.12 Other Endpoints and Analyses—Feasibility of Self-Administration of CAM2029

For patients opting for self- or partner-administration of CAM2029, the proportion of patients/partners judged competent by the trial personnel out of those trying will be presented. The number and proportion of patients requiring 1, 2, or 3 attempts until success or failure after maximum of 3 attempts will be provided.

For patients/partners declared competent after at most 3 attempts, any unsuccessful administration will be listed.

### 8.13 Extent of Exposure and Treatment Compliance

Exposure and compliance will be calculated per patient and summarized descriptively.

The duration of exposure in weeks will be summarized by means of descriptive statistics using the safety analysis set. The number of injections received may also be summarized.

The number of patients with dose delays or permanent discontinuation of IMP and the reasons will be summarized by treatment group and all dosing data will be listed.

### 8.14 Interim Analyses

The interim analysis for overall survival performed in connection to the primary PFS analysis is described in Section [000659].

### 16 References

1. Oronsky B, Ma P C, Morgensztern D, Carter C A. Nothing But NET: A Review of Neuroendocrine Tumors and Carcinomas. Neoplasia. 2017; 19(12):991-1002.
2. Nagtegaal ID, Odze R D, Klimstra D, Paradis V, Rugge M, Schirmacher P, et al. The 2019 WHO classification of tumours of the digestive system. Histopathology. 2020; 76(2):182-8.
3. Yao J C, Hassan M, Phan A, Dagohoy C, Leary C, Mares J E, et al. One hundred years after “carcinoid”: epidemiology of and prognostic factors for neuroendocrine tumors in 35,825 cases in the United States. J Clin Oncol. 2008; 26(18):3063-72.
4. Dasari A, Shen C, Halperin D, Zhao B, Zhou S, Xu Y, et al. Trends in the Incidence, Prevalence, and Survival Outcomes in Patients With Neuroendocrine Tumors in the United States. JAMA Oncol. 2017; 3(10):1335-42.
5. Modlin I M, Lye K D, Kidd M. A 5-decade analysis of 13,715 carcinoid tumors. Cancer. 2003; 97(4):934-59.
6. Boudreaux J P, Klimstra D S, Hassan M M, Woltering E A, Jensen R T, Goldsmith S J, et al. The NANETS consensus guideline for the diagnosis and management of neuroendocrine tumors: well-differentiated neuroendocrine tumors of the Jejunum, Ileum, Appendix, and Cecum. Pancreas. 2010; 39(6):753-66.
7. Strosberg J, Gardner N, Kvols L. Survival and prognostic factor analysis in patients with metastatic pancreatic endocrine carcinomas. Pancreas. 2009; 38(3):255-8.
8. Ter-Minassian M, Chan J A, Hooshmand S M, Brais L K, Daskalova A, Heafield R, et al. Clinical presentation, recurrence, and survival in patients with neuroendocrine tumors: results from a prospective institutional database. Endocr Relat Cancer. 2013; 20(2):187-96.
9. Yao J C, Lombard-Bohas C, Baudin E, Kvols L K, Rougier P, Ruszniewski P, et al. Daily oral everolimus activity in patients with metastatic pancreatic neuroendocrine tumors after failure of cytotoxic chemotherapy: a phase II trial. J Clin Oncol. 2010; 28(1):69-76.
10. Yao J C, Shah M H, Ito T, Bohas C L, Wolin E M, Van Cutsem E, et al. Everolimus for advanced pancreatic neuroendocrine tumors. N Engl J Med. 2011; 364(6):514-23.
11. Yao J C, Pavel M, Lombard-Bohas C, Van Cutsem E, Voi M, Brandt U, et al. Everolimus for the Treatment of Advanced Pancreatic Neuroendocrine Tumors: Overall Survival and Circulating Biomarkers From the Randomized, Phase III RADIANT-3 Study. J Clin Oncol. 2016; 34(32):3906-13.
12. Pavel M E, Wiedenmann B. Novel therapeutic agents for the treatment of gastroenteropancreatic neuroendocrine tumors. Horm Metab Res. 2011; 43(12):844-53.
13. Kwekkeboom D J, Krenning E P. Peptide Receptor Radionuclide Therapy in the Treatment of Neuroendocrine Tumors. Hematol Oncol Clin North Am. 2016; 30(1):179-91.
14. Rinke A, Muller H H, Schade-Brittinger C, Klose K J, Barth P, Wied M, et al. Placebo-Controlled, Double-Blind, Prospective, Randomized Study on the Effect of Octreotide LAR in the Control of Tumor Growth in Patients With Metastatic Neuroendocrine Midgut Tumors: A Report From the PROMID Study Group. J Clin Oncol. 2009; 27(28):4656-63.
15. Caplin M E, Pavel M, Cwikla J B, Phan A T, Raderer M, Sedliekovi E, et al. Lanreotide in metastatic enteropancreatic neuroendocrine tumors. N Engl J Med. 2014; 371(3):224-33.
16. Strosberg J, E1-Haddad G, Wolin E, Hendifar A, Yao J, Chasen B, et al. Phase 3 Trial of (177)Lu-Dotatate for Midgut Neuroendocrine Tumors. N Engl J Med. 2017; 376(2):125-35.
17. Broder M S, Beenhouwer D, Strosberg J R, Neary M P, Cherepanov D. Gastrointestinal neuroendocrine tumors treated with high dose octreotide-LAR: a systematic literature review.
World J Gastroenterol. 2015; 21(6):1945-55.
18. Eriksson B, Renstrup J, Imam H, Oberg K. High-dose treatment with lanreotide of patients with advanced neuroendocrine gastrointestinal tumors: clinical and biological effects. Ann Oncol. 1997; 8(10):1041-4.
19. Faiss S, Rath U, Mansmann U, Caird D, Clemens N, Riecken E O, et al. Ultra-high-dose lanreotide treatment in patients with metastatic neuroendocrine gastroenteropancreatic tumors. Digestion. 1999; 60(5):469-76.
20. Welin S, Janson E, Sundin A, Stridsberg M, Lavenius E, Granberg D, et al. High-dose treatment with a long-acting somatostatin analogue in patients with advance carcinoid tumours. Eur J Endocrinol. 2004; 151(1):107-12.
21. Efficacy and Safety Study in Pancreatic or Midgut Neuroendocrine Tumours Having Progressed Radiologically While Previously Treated With Lanreotide Autogel® 120 mg (CLARINET FORTE), NCT02651987 [Available from: [Internet] clinicaltrials.gov/ct2/show/NCT02651987].
22. Pavel M, Cwik J B, Lombard-Bohas C, Borbath I, Shah T, Pape U F, et al. 1162MO Efficacy and safety of lanreotide autogel (LAN) 120 mg every 14 days in progressive pancreatic or midgut neuroendocrine tumours (NETs): CLARINET FORTE study results [Available from: [Internet] annalsofoncology.org/action/showPdf?pii=S0923-7534%2820%2941371-7].
23. Study to Evaluate the Efficacy and Safety of Lutathera in Patients With Grade 2 and Grade 3 Advanced GEP-NET (NETTER-2), NCT03972488 [Available from: [Internet] clinicaltrials.gov/ct2/show/NCT03972488].
24. Lau S C, Abdel-Rahman O, Cheung W Y. Improved survival with higher doses of octreotide long-acting release in gastroenteropancreatic neuroendocrine tumors. Med Oncol. 2018; 35(9):123.
25. Chadha M K, Lombardo J, Mashtare T, Wilding G E, Litwin A, Raczyk C, et al. High-dose octreotide acetate for management of gastroenteropancreatic neuroendocrine tumors. Anticancer Res. 2009; 29(10):4127-30.
26. Ferolla P, Faggiano A, Grimaldi F, Ferone D, Scarpelli G, Ramundo V, et al. Shortened interval of long-acting octreotide administration is effective in patients with well-differentiated neuroendocrine carcinomas in progression on standard doses. J Endocrinol Invest. 2012; 35(3):326-31.
27. Sharp A J, Hayes A R, Grossman A. High-dose Somatostatin Analogues for Progressive Neuroendocrine Tumours. European endocrinology. 2020; 16(2):93-5.
28. Diamantopoulos L, Laskaratos F M, Kalligeros M, Shah R, Navalkissoor S, Gnanasegaran G, et al. Antiproliferative Effect Of Above-Label Doses Of Somatostatin Analogs For The Management Of Gastroenteropancreatic Neuroendocrine Tumors. Neuroendocrinology. 2020.
30. Sandostatin LAR 30 mg powder and solvent for suspension for injection. SmPC 2020 [Available from: [Internet] medicines.org.uk/emc/product/7828].
31. Rinke A, Wittenberg M, Schade-Brittinger C, Aminossadati B, Ronicke E, Gress T M, et al. Placebo-Controlled, Double-Blind, Prospective, Randomized Study on the Effect of Octreotide LAR in the Control of Tumor Growth in Patients with Metastatic Neuroendocrine Midgut Tumors (PROMID): Results of Long-Term Survival. Neuroendocrinology. 2017; 104(1):26-32.
32. FDA Guidance for Industry. Clinical Trial Endpoints for the Approval of Cancer Drugs and Biologics. December 2018 [Available from: [Internet] fda.gov/media/71195/download].
33. Kulke M H, Siu L L, Tepper J E, Fisher G, Jaffe D, Haller D G, et al. Future directions in the treatment of neuroendocrine tumors: consensus report of the National Cancer Institute Neuroendocrine Tumor clinical trials planning meeting. J Clin Oncol. 2011; 29(7):934-43.
34. Somatuline Autogel 60 mg, solution for injection in a prefilled syringe.SmPC 2019 [Available from: [Internet] medicines.org.uk/emc/medicine/25104].
35. Sandostatin® LAR® Novartis. Sandostatin LAR Depot U S Package Insert 2019 [Available from: [Internet] accessdata.fda.gov/drugsatfda_docs/label/2019/021008s043lbl.pdf].
36. Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45(2):228-47.
37. Aaronson N K, Ahmedzai S, Bergman B, Bullinger M, Cull A, Duez N J, et al. The European Organization for Research and Treatment of Cancer QLQ-C30: a quality-of-life instrument for use in international clinical trials in oncology. J Natl Cancer Inst. 1993; 85(5):365-76.
38. Fayers P M, Aaronson N K, Bjordal K, Groenvold M, Curran D, Bottomley A. On behalf of the EORTC Quality of Life Group. The EORTC QLQ-C30 Scoring Manual (3rd Edition). Published by: European Organisation for Research and Treatment of Cancer, Brussels 2001.
39. Yadegarfar G, Friend L, Jones L, Plum L M, Ardill J, Taal B, et al. Validation of the EORTC QLQ-GINET21 questionnaire for assessing quality of life of patients with gastrointestinal neuroendocrine tumours. Br J Cancer. 2013; 108(2):301-10.
40. Ware J E, Jr., Sherbourne C D. The MOS 36-item short-form health survey (SF-36). I. Conceptual framework and item selection. Med Care. 1992; 30(6):473-83.
41. Guy W. 1976. Clinical Global Impressions. ECDEU Assessment Manual for Psychopharmacology-Revised. Rockville, M D: U.S. Department of Health, Education, and Welfare; Public Health Service, Alcohol; Drug Abuse, and Mental Health Administration; National Institute of Mental Health; Psychopharmacology Research Branch; Division of Extramural Research Programs. pp 218-222 [Available from: [Internet] archive.org/details/ecdeuassessmentm1933guyw/page/218/mode/2up].
42. Atkinson M J, Sinha A, Hass S L, Colman S S, Kumar R N, Brod M, et al. Validation of a general measure of treatment satisfaction, the Treatment Satisfaction Questionnaire for Medication (TSQM), using a national panel study of chronic disease. Health and quality of life outcomes. 2004; 2:12.
43. Oken M M, Creech R H, Tormey D C, Horton J, Davis T E, McFadden E T, et al. Toxicity and response criteria of the Eastern Cooperative Oncology Group. Am J Clin Oncol. 1982; 5(6):649-55.
44. Salvatori R, Nachtigall L B, Cook D M, Bonert V, Molitch M E, Blethen S, et al. Effectiveness of self- or partner-administration of an extended-release aqueous-gel formulation of lanreotide in lanreotide-naïve patients with acromegaly. Pituitary. 2010; 13(2):115-22.
45. Salvatori R, Woodmansee W W, Molitch M, Gordon M B, Lomax K G. Lanreotide extended-release aqueous-gel formulation, injected by patient, partner or healthcare provider in patients with acromegaly in the United States: 1-year data from the SODA registry. Pituitary. 2014; 17(1):13-21.
46. Haybittle J L. Repeated assessment of results in clinical trials of cancer treatment. Br J Radiol. 1971; 44(526):793-7.
47. Peto R, Pike M C, Armitage P, Breslow N E, Cox D R, Howard S V, et al. Design and analysis of randomized clinical trials requiring prolonged observation of each patient. I. Introduction and design. Br J Cancer. 1976; 34(6):585-612.
48. FDA. Guidance for Industry. Population Pharmacokinetics. Draft guidance. July 2019 [Available from: [Internet] fda.gov/media/128793/download].
49. ICH Harmonised Tripartite Guideline E6 (R2): Guideline for Good Clinical Practice [Available from: [Internet] ema.europa.eu/en/documents/scientific-guideline/ich-e-6-r2-guideline-good-clinical-practice-step-5_en.pdf].
50. World Medical Association (WMA). Declaration of Helsinki: Ethical principles for medical research involving human subjects [Available from: [Internet] wma.net/policies-post/wma-declaration-of-helsinki-ethical-principles-for-medical-research-involving-human-subjects/].

### 17 Appendices

### 17.1 Appendix 1 Dose Delay and Discontinuation Guidance

### 17.1.1 General Management Guidelines for Adverse Drug Reactions

TABLE 10
IMP management guidelines for suspected adverse drug reactions

| Toxicity severity | Recommendations |
|---|---|
| CTCAE grade ≤2 (mild | No action required |
| or moderate)^{a} | |
| CTCAE grade ≥3 | Delay treatment until ADR resolves or |
| (severe)^{a} | improves to CTCAE grade ≤2 (mild or |
| | moderate). |
| | If ADR resolves or improves within 28 days, |
| | consider restarting IMP. There is no option |
| | to reduce IMP dose levels in the present trial |
| | and if the ADRs are expected to re-occur |
| | upon re-challenge, consider discontinuing |
| | IMP permanently. |
| | If ADR does not resolve or improve to CTCAE |
| | grade ≤2 (mild or moderate) within 28 days, |
| | IMP must be permanently discontinued. |

ADR: adverse drug reaction;
CTCAE: Common Terminology Criteria for Adverse Events;
IMP: investigational medicinal product
The above guidance generally applies to ADRs. For changes in liver function tests (see Section [000427]) and for changes in QT intervals, see instructions in Table 11. Note that these are only recommendations. The nature of the ADRs must be taken into consideration.
^{a}If CTCAE grading does not exist for an ADR, the severities of mild, moderate and severe, corresponding to Grades 1 through 3, may be used

### 17.1.2 Follow-Up on Potential Drug-Induced Liver Injury Cases

Patients who were discontinued due to hepatic-related discontinuation criteria in Section [000427] must be followed up. The evaluation should include laboratory tests, detailed history, physical assessment, and the possibility of new liver lesions, obstructions/compressions, etc.
Laboratory tests should include ALT, AST, albumin, creatine kinase, total bilirubin, direct and indirect bilirubin, gamma glutamyl transferase, prothrombin time/international normalized ratio and alkaline phosphatase
A detailed history should be collected, including relevant information such as review of ethanol, concomitant medications, herbal remedies, supplement consumption, history of any pre-existing liver conditions or risk factors
Further testing for acute hepatitis A, B, C, or E infection and liver imaging (e.g. biliary tract potential metastases) may be warranted
Obtain PK sample in connection with blood sampling for laboratory tests
Additional testing for other hepatotropic viral infection (cytomegalovirus, Epstein-Barr virus, herpes simplex virus), autoimmune hepatitis or liver biopsy may be considered as clinically indicated or after consultation with specialist/hepatologist

All cases confirmed on repeat testing meeting the laboratory criteria defined above, with no other identified alternative cause for liver function test abnormalities should be considered as “medically significant”, thus, meeting the definition of SAE (Section [000537]) and reported as an SAE using the term “potential drug-induced liver injury”. All events should be followed up with the outcome clearly documented.

### 17.1.3 Specific Management Recommendations for Cases of QTcF Prolongation

TABLE 11
Dose interruptions for QTcF prolongation

| Severity | Dose adjustment and management recommendation |
|---|---|
| For all grades | Check the quality of the ECG and the QT value and repeat if needed. |
| | Analyze serum electrolytes (potassium, calcium, phosphorus, |
| | magnesium). If electrolytes are significantly outside of normal range, |
| | correct with supplements or appropriate therapy as soon as possible, and |
| | repeat electrolyte analyses until documented as normal. |
| | Review concomitant medication usage for the potential to |
| | prolong the QT interval. |
| | Check compliance with correct dose and administration of IMP. |
| | Consider collecting a time-matched PK sample; record date |
| | and time of last IMP intake. |
| Grade 1 | No dose interruption recommended. |
| Average QTcF: | |
| Males: 450 msec to | |
| 480 msec | |
| Females: 470 msec to | |
| 480 msec | |
| Grade 2 | Pause IMP. |
| Average QTcF: 481 msec | Perform repeated triplicate ECGs approximately 1 hour after the first |
| to 500 msec | QTcF of ≥481 msec. |
| | If QTcF <481 msec, restart IMP. |
| | If QTcF remains ≥481 msec, repeat ECG as clinically indicated until the |
| | QTcF returns to <481 msec. Restart IMP. |
| | If QTcF ≥481 msec remains or recurs, consider discontinuing IMP. |
| | Repeat ECGs 7 days and 14 days after dose resumption (then as clinically |
| | indicated) for any patient who had therapy interrupted due to |
| | QTcF ≥481 msec. |
| Grade 3 | Perform repeated triplicate ECGs within 1 hour of the first QTcF |
| Average | of ≥501 msec or >60 msec change from baseline. |
| QTcF: ≥501 msec | If QTcF remains ≥501 msec or if the change from baseline |
| or >60 msec change | remains >60 msec, discontinue IMP. |
| from baseline or | Transmit ECG immediately and confirm prolongation/abnormalities with |
| Grade 4 | central assessment. |
| Torsades de pointes or | Obtain local cardiologist (or qualified specialist) consultation and repeat |
| polymorphic ventricular | cardiac monitoring as indicated until the QTcF returns to ≤500 msec. |
| tachycardia, or | Repeat ECG as clinically indicated until the QTcF returns to <481 msec. |
| signs/symptoms of serious | |
| arrhythmia | |

ECG: electrocardiogram;
IMP: investigational medicinal product;
PK: pharmacokinetic;
QTcF: QTc interval corrected by Fridericia's formula

Although the disclosure has been described with reference to embodiments and examples, it should be understood that numerous and various modifications can be made without departing from the spirit of the disclosure. Accordingly, the disclosure is limited only by the following claims. All publications, patents, and patent applications cited in this specification are incorporated herein by reference as if each such publication, patent or patent application were specifically and individually indicated to be incorporated herein by reference

### Example 2: Pharmacokinetic Model

The dosage regimes of CAM2029 used herein and the corresponding octreotide plasma exposure (Cmax, AUC etc) for treatment of patients with GEP-NET were predicted by a population pharmacokinetic (PK) model of octreotide. The PK model has been developed by nonlinear mixed effects modelling using the NONMEM software as described by Beal S L, Sheiner L B, Boeckmann A J, Bauer R J. NONMEM User's Guides. (1989-2014) Icon Development Solutions, Ellicott City, MD, USA. (2014), and based on octreotide plasma concentrations from healthy subjects receiving repeated monthly doses of 10, 20 or 30 mg CAM2029 (data from clinical phase 1 study using CAM2029 reported in Tiberg et al., British J. Clin. Pharmacol. 80(3):460-472 (2015)). The PK parameters maximum plasma concentration (Cₘₐₓ), area under the plasma concentration-time curve (AUC) and average concentration during a dosage interval at steady state (C_{avg}) used herein are based on the population PK model. The plasma exposure in subject used for the PK model and used in the clinical studies were obtained as described by Karnes et al, Journal of Chromatography B, 879 (2011), 2081-2088. The model resulted in the predicted doses disclosed in FIG. 2, and lead to the dosing regime disclosed herein.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A method of treating at least one neuroendocrine tumor comprising, administering to a patient in need thereof a lipid composition comprising 20 mg of octreotide or a pharmaceutically acceptable salt thereof, wherein the lipid composition is administered to the patient once every two weeks.

2. The method of claim 1, wherein the lipid composition is administered as a unit dose.

3. The method of claim 1, wherein the at least one neuroendocrine tumor is a gastroenteropancreatic neuroendocrine tumor.

4. The method of claim 1, wherein the octreotide or a salt thereof is octreotide chloride.

5. The method of claim 4, wherein the octreotide chloride is the sole active agent in the lipid composition.

6. The method of claim 1, wherein the lipid composition further comprises glycerol dioleate, phosphatidylcholine, and ethanol.

7. The method of claim 6, wherein the lipid composition further comprises propylene glycol.

8. The method of claim 6, wherein the lipid composition further comprises EDTA.

9. The method of claim 8, wherein the lipid composition further comprises ethanolamine and/or diethanolamine.

10. The method of claim 1, wherein at least 85 wt % of the lipid composition consists of octreotide chloride, glycerol dioleate, phosphatidylcholine, ethanol, and propylene glycol.

11. The method of claim 1, wherein at least 86 wt % of the lipid composition consists of octreotide chloride, glycerol dioleate, phosphatidylcholine, ethanol, propylene glycol, EDTA, and the lipid composition has a water content of less than 1.0 wt % at the release of the product for sale.

12. The method of claim 1, comprising administering the lipid composition by a syringe, a pre-filled syringe, an autoinjector, or a pen injector.

13. The method of claim 1, administering the lipid composition by subcutaneous injection.

14. The method of claim 1, comprising administering the lipid composition not more than once every two weeks, and in a volume of about 1 mL.

15. The method of claim 1, wherein the patient has a maximum blood plasma concentration (Cₘₐₓ) of octreotide (at steady state) of less than 45 ng/ml for each administration.

16. The method of claim 1, wherein the patient has a maximum blood plasma concentration (Cₘₐₓ) of octreotide (at steady state) of 3-45 ng/ml for each administration.

17. The method of claim 1, wherein the patient has an average blood plasma concentration (C_{AV}) of octreotide (at steady state) of 3-24 ng/ml for each administration.

18. The method of claim 1, wherein the patient has an average blood plasma concentration (C_{AV}) of octreotide (at steady state) of 4-20 ng/ml for each administration.

19. The method of claim 1, wherein the patient has a plasma concentration AUC of octreotide (at steady state) of 1300-6700 ng*h/ml for each administration.

20. The method of claim 1, wherein the patient has a plasma concentration AUC of octreotide (at steady state) of 1550-3500 ng*h/ml for each administration.

21. The method of claim 1, wherein administering the lipid composition comprising 20 mg of octreotide provides a drug depot comprising octreotide in the patient, said depot providing a release of octreotide to the patient over about two weeks and wherein the patient has blood plasma levels of octreotide of at least about 3 ng/ml for each administration.

22. The method of claim 21, wherein the depot is in the subcutaneous tissue of the patient.

23. The method of claim 1, wherein the lipid composition is administered to the patient once every two weeks until the patient experiences progressive disease (PD), determined radiologically according to Response Evaluation Criteria in Solid Tumors version 1.1 (RECIST 1.1), whereupon the lipid composition is optionally administered to the patient once every week.

24. The method of claim 1, wherein the method provides a progression-free survival of at least 16 months.

25. The method of claim 1, wherein the method provides an improved progression-free survival (PFS) compared to Sandostatin® LAR®.

26. The method of claim 1, wherein the method provides an improved progression-free survival (PFS) compared to placebo or Sandostatin® LAR®, and/or increased or about the same progression-free survival as Somatuline® Depot (Somatuline® Autogel), wherein the progression-free survival is determined radiologically according to Response Evaluation Criteria in Solid Tumors version 1.1 (RECIST 1.1).

27. The method of claim 1, wherein the method provides an improved overall response rate (ORR) compared to Sandostatin® LAR® and/or improved or about the same ORR as Somatuline® Depot (Somatuline® Autogel) as determined according to Response Evaluation Criteria in Solid Tumors version 1.1 (RECIST 1.1).

28. The method of claim 27, wherein the overall response rate is selected from the group consisting of complete response and/or partial response.

29. The method of claim 1, wherein the method provides an improved disease control rate (DCR) compared to Sandostatin® LAR® and/or improved or about the same DCR as Somatuline® Depot (Somatuline® Autogel) as determined according to Response Evaluation Criteria in Solid Tumors version 1.1 (RECIST 1.1).

30. The method of claim 23, wherein the patient has a maximum blood plasma concentration (Cₘₐₓ) of octreotide (at steady state) of less than 45 ng/ml for each once every administration.

31. The method of claim 23, wherein the patient has a maximum blood plasma concentration (Cₘₐₓ) of octreotide (at steady state) of 3-45 ng/ml for each once every administration.

32. The method of claim 23, wherein the patient has an average blood plasma concentration (C_{AV}) of octreotide (at steady state) of 6-40 ng/ml for each once every week administration.

33. The method of claim 23, wherein the patient has an average blood plasma concentration (C_{AV}) of octreotide (at steady state) of 4-20 ng/ml for each once every week administration.

34. The method of claim 23, wherein the patient has a plasma concentration AUC of octreotide (at steady state) of 1000-6700 ng*h/ml for each once every week administration.

35. The method of claim 23, wherein the patient has a plasma concentration AUC of octreotide (at steady state) of 1550-3500 ng*h/ml for each once every week administration.

36. The method of claim 23, wherein administering the lipid composition comprising 20 mg of octreotide provides a drug depot comprising octreotide in the patient, said depot providing a release of octreotide to the patient over about 1 week and wherein the patient has blood plasma levels of octreotide of at least about 5 ng/ml for each once every week administration.

37. A lipid composition comprising 20 mg of octreotide or a pharmaceutically acceptable salt thereof for use in treating at least one neuroendocrine tumor, and wherein the composition is administered to a patient once every two weeks.

38-69. (canceled)

70. A pre-filled syringe comprising the composition of claim 37.

71. An autoinjector comprising a glass compartment containing the composition of claim 37.

72. (canceled)

73. A kit for the administration of octreotide or a pharmaceutically acceptable salt thereof having one or more containers comprising 20 mg of octreotide or a pharmaceutically acceptable salt thereof, wherein the octreotide or a pharmaceutically acceptable salt in the one or more containers is administered according to the method of claim 1.
